(19) United States
(12) Patent Application Publication
(10) Pub. No.: US 2026/0194811 A1
(43) Pub. Date: Jul. 9, 2026

(54) RESIST COMPOSITION, RESIST PATTERN FORMATION METHOD, COMPOUND, AND ACID GENERATOR

(71) Applicant: Tokyo Ohka Kogyo Co., Ltd., Kawasaki-shi (JP)

(72) Inventors: Yoshitaka Komuro, Kawasaki-shi (JP); Kazufumi Sato, Kawasaki-shi (JP)

(21) Appl. No.: 19/133,190

(22) PCT Filed: Nov. 28, 2023
(86) PCT No.: PCT/JP2023/042466

§ 371 Date: May 27, 2025

(30) Foreign Application Priority Data
Nov. 28, 2022 (JP) 2022-189368

(51) Int. Cl. G03F 7/004; C07C 309/58; C07C 381/12; C07D 307/00; C07D 311/78; G03F 7/038; G03F 7/039

(52) U.S. Cl. CPC G03F 7/0045; C07C 309/58; C07C 381/12; C07D 307/00; C07D 311/78; G03F 7/0382; G03F 7/0388; G03F 7/0397

(57) ABSTRACT

A resist composition which generates an acid upon light exposure and whose solubility in a developing solution is changed by an action of the acid. The resist composition includes a resin component whose solubility in a developing solution is changed by the action of the acid, and an acid generator component which generates an acid upon light exposure, in which the acid generator component contains a compound represented by Formula (b0). In General Formula (b0), Ar represents an aromatic ring, Rf⁰ represents a fluorinated alkyl group or a fluorine atom, L⁰ represents a divalent linking group, Yb⁰ represents a cyclic group, Rb⁰ represents an organic group, n01 represents an integer of 1 or greater, n02 represents an integer of 0 or greater, M^{m+} represents an m-valent cation, and when L⁰-Yb⁰ is —C(═O)—O—Yb⁰, Yb⁰¹ represents an alicyclic group

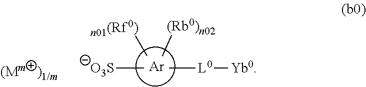

### TECHNICAL FIELD

The present invention relates to a resist composition, a resist pattern formation method, a compound, and an acid generator.

Priority is claimed on Japanese Patent Application No. 2022-189368, filed Nov. 28, 2022, the content of which is incorporated herein by reference.

### BACKGROUND ART

In recent years, in the production of semiconductor elements and liquid crystal display elements, advances in lithography technologies have led to a rapid progress in the field of pattern fining. Typically, these pattern fining technologies involve shortening the wavelength (increasing the energy) of the light source for exposure.

A resist material is required to have lithography characteristics such as resolution that enables reproduction of a fine-sized pattern, and sensitivity to these types of exposure light sources.

As a resist material that satisfies these requirements, a chemically amplified resist composition that contains a base material component whose solubility in a developing solution is changed by an action of an acid, and an acid generator component that generates an acid upon light exposure has been used in the related art.

In a chemically amplified resist, an acid generator in which a carbon atom near a sulfonic acid group is perfluorinated has been put into practical use as an acid generator which provides an acid having sufficient acid strength for deprotection. Meanwhile, in recent years, with the recent increase in environmental awareness, a low-fluorinated sulfonate having a reduced fluorine content has been suggested for the purpose of reducing the environmental burden. However, even in a low-fluorinated acid generator, in order to ensure the acid strength, the introduction of a fluorine atom into a carbon atom near a sulfonic acid group is unavoidable. For example, Patent Document 1 describes an acid generator in which a carbon atom adjacent to a sulfonic acid group is fluorinated.

### CITATION LIST

### Patent Document

Patent Document 1: Japanese Unexamined Patent Application, First Publication No. 2018-92159

### SUMMARY OF INVENTION

### Technical Problem

With a further increase in environmental awareness in the future, there is a possibility that production and use of an acid generator in which a fluorine atom is introduced into a carbon atom adjacent to a sulfonic acid is regulated. However, in an acid generator of the related art such as the acid generator described in Patent Document 1, in a case where a carbon atom adjacent to a sulfonic acid group is not fluorinated, it is difficult to obtain sufficient acid strength.

Meanwhile, miniaturization of patterns has been rapidly promoted by further advances in lithography technologies. Therefore, a resist composition having satisfactory lithography characteristics such as roughness while maintaining satisfactory sensitivity has been required.

The present invention has been made in consideration of the above-described circumstances, and an object thereof is to provide a resist composition containing an acid generator in which a carbon atom adjacent to sulfonic acid is not fluorinated and having satisfactory lithography characteristics, a resist pattern formation method using the resist composition, a compound that can be used as an acid generator of the resist composition, and an acid generator containing the compound.

### Solution to Problem

In order to solve the above-described problems, the present invention has adopted the following configurations.

That is, according to a first aspect of the present invention, there is provided a resist composition which generates an acid upon light exposure and whose solubility in a developing solution is changed by an action of the acid, the resist composition including: a resin component (A1) whose solubility in a developing solution is changed by the action of the acid; and an acid generator component (B) which generates an acid upon light exposure, in which the acid generator component (B) contains a compound (B0) represented by General Formula (b0).

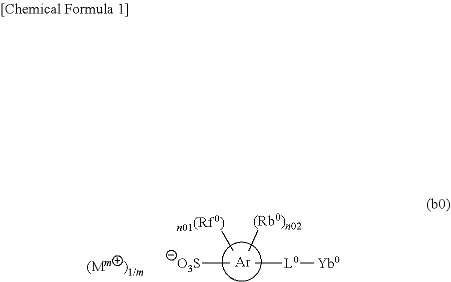

[In the formula, Ar represents an aromatic ring. Rf⁰ represents a fluorinated alkyl group having 1 to 5 carbon atoms or a fluorine atom. L⁰ represents a divalent linking group having —C(═O)—O—, —O—C(═O)—, or —O—S(═O)₂—. Yb⁰ represents a cyclic group. Rb⁰ represents an organic group. n01 represents an integer of 1 or greater as long as a valence is allowed. n02 represents an integer of 0 or greater as long as a valence is allowed. Here, in a case where L⁰-Yb⁰ represents —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of an aromatic ring is substituted with an alkyl group or an alkoxy group. In a case where n01 represents 2 or greater, a plurality of Rf⁰'s may be the same as or different from each other. In a case where n02 represents 2 or greater, a plurality of Rb⁰'s may be the same as or different from each other. m represents an integer of 1 or greater, and M^{m+} represents an m-valent cation.]

According to a second aspect of the present invention, there is provided a resist pattern formation method including: a step of forming a resist film on a support using the resist composition according to the first aspect, a step of exposing the resist film to light; and a step of developing the resist film exposed to light to form a resist pattern.

According to a third aspect of the present invention, there is provided a compound represented by General Formula (b0).

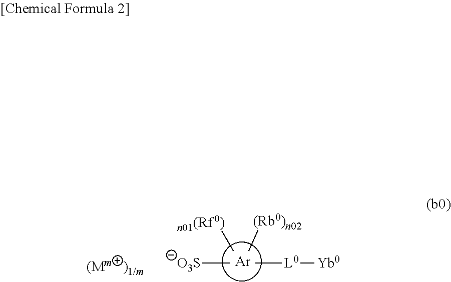

[In the formula, Ar represents an aromatic ring. Rf⁰ represents a fluorinated alkyl group having 1 to 5 carbon atoms or a fluorine atom. L⁰ represents a divalent linking group having —C(═O)—O—, —O—C(═O)—, or —O—S(═O)₂—. Yb⁰ represents a cyclic group. Rb⁰ represents an organic group. n01 represents an integer of 1 or greater as long as a valence is allowed. n02 represents an integer of 0 or greater as long as a valence is allowed. Here, in a case where L⁰-Yb⁰ represents —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of an aromatic ring is substituted with an alkyl group or an alkoxy group. In a case where n01 represents 2 or greater, a plurality of Rf⁰'s may be the same as or different from each other. In a case where n02 represents 2 or greater, a plurality of Rb⁰'s may be the same as or different from each other. m represents an integer of 1 or greater, and M^{m+} represents an m-valent cation.]

According to a fourth aspect of the present invention, there is provided an acid generator including: the compound according to the third aspect.

### Advantageous Effects of Invention

According to the present invention, it is possible to provide a resist composition containing an acid generator in which a carbon atom adjacent to sulfonic acid is not fluorinated and having satisfactory lithography characteristics, a resist pattern formation method using the resist composition, a compound that can be used as an acid generator of the resist composition, and an acid generator containing the compound.

### DESCRIPTION OF EMBODIMENTS

In the present specification and the scope of the present claims, the term “aliphatic” is a relative concept used with respect to “aromatic” and defines a group or compound that has no aromaticity.

The term “alkyl group” includes a linear, branched, or cyclic monovalent saturated hydrocarbon group unless otherwise specified. The same applies to the alkyl group in an alkoxy group.

The term “alkylene group” includes a linear, branched, or cyclic divalent saturated hydrocarbon group unless otherwise specified.

Examples of “halogen atom” include a fluorine atom, a chlorine atom, a bromine atom, and an iodine atom.

The term “constitutional unit” indicates a monomer unit constituting a polymer compound (a resin, a polymer, or a copolymer).

The expression “may have a substituent” includes both a case where a hydrogen atom (—H) is substituted with a monovalent group and a case where a methylene (—CH₂—) group is substituted with a divalent group.

The term “light exposure” is a general concept for irradiation with radiation.

The term “acid decomposable group” indicates a group having acid decomposability in which at least a part of a bond in the structure of the acid decomposable group can be cleaved by the action of an acid.

Examples of the acid decomposable group whose polarity is increased by the action of an acid include groups which are decomposed by the action of an acid to generate a polar group.

Examples of the polar group include a carboxy group, a hydroxyl group, an amino group, and a sulfo group (—SO₃H).

More specific examples of the acid decomposable group include a group in which the above-described polar group has been protected by an acid dissociable group (such as a group in which a hydrogen atom of the OH-containing polar group has been protected by an acid dissociable group).

Here, the term “acid dissociable group” indicates both a group (i) having an acid dissociation property in which a bond between the acid dissociable group and an atom adjacent to the acid dissociable group can be cleaved by the action of an acid and a group (ii) in which some bonds are cleaved by the action of an acid, a decarboxylation reaction occurs, and thus the bond between the acid dissociable group and the atom adjacent to the acid dissociable group can be cleaved.

It is necessary that the acid dissociable group that constitutes the acid decomposable group is a group which exhibits a lower polarity than that of the polar group generated by the dissociation of the acid dissociable group. Thus, in a case where the acid dissociable group is dissociated by the action of an acid, a polar group exhibiting a higher polarity than that of the acid dissociable group is generated so that the polarity is increased. As a result, the polarity of an entire component (A1) is increased. Due to the increase in the polarity, the solubility in a developing solution is relatively changed such that the solubility is increased in a case where the developing solution is an alkali developing solution and the solubility is decreased in a case where the developing solution is an organic developing solution.

The term “base material component” denotes an organic compound having a film-forming ability. Organic compounds used as the base material component are classified into non-polymers and polymers. As the non-polymers, typically non-polymers having a molecular weight of 500 or greater and less than 4000 (hereinafter, referred to as “low-molecular-weight compounds”) are used. Hereinafter, “resin”, “polymer compound”, or “polymer” indicates a polymer having a molecular weight of 1000 or greater. As the molecular weight of the polymer, the weight-average molecular weight in terms of polystyrene according to gel permeation chromatography (GPC) is used.

The expression “constitutional unit to be derived” denotes a constitutional unit formed by cleavage of a multiple bond between carbon atoms, for example, an ethylenic double bond.

In “acrylic acid ester”, the hydrogen atom bonded to the carbon atom at the α-position may be substituted with a substituent. The substituent (R^{αx}) that substitutes the hydrogen atom bonded to the carbon atom at the α-position is an atom other than the hydrogen atom or a group. Further, itaconic acid diester in which the substituent (R^{αx}) has been substituted with a substituent having an ester bond or α-hydroxyacryl ester in which the substituent (RaX) has been substituted with a hydroxyalkyl group or a group obtained by modifying a hydroxyl group thereof can be described as acrylic acid ester. Further, the carbon atom at the α-position of acrylic acid ester indicates the carbon atom to which the carbonyl group of acrylic acid is bonded, unless otherwise specified.

Hereinafter, acrylic acid ester in which the hydrogen atom bonded to the carbon atom at the α-position has been substituted with a substituent is also referred to as α-substituted acrylic acid ester.

The concept “derivative” includes those obtained by substituting a hydrogen atom at the α-position of a target compound with another substituent such as an alkyl group or a halogenated alkyl group, and derivatives thereof. Examples of the derivatives thereof include those obtained by substituting a hydrogen atom of a hydroxyl group of a target compound, in which the hydrogen atom at the α-position may be substituted with a substituent, with an organic group, and those obtained by bonding a substituent other than a hydroxyl group to a target compound in which the hydrogen atom at the α-position may be substituted with a substituent. Further, the α-position denotes the first carbon atom adjacent to a functional group unless otherwise specified.

Examples of the substituent that substitutes the hydrogen atom at the α-position of hydroxystyrene include those for R^{αx}.

In the present specification and the scope of the present claims, asymmetric carbons may be present and enantiomers or diastereomers may be present depending on the structures of the chemical formulae. In this case, these isomers are represented by one chemical formula. These isomers may be used alone or in the form of a mixture.

### (Resist Composition)

The resist composition according to the present embodiment is a resist composition which generates an acid upon light exposure and whose solubility in a developing solution is changed by the action of the acid.

Such a resist composition contains a base material component (A) (hereinafter, also referred to as “component (A)”) whose solubility in a developing solution is changed by the action of the acid, and an acid generator component (B) that generates an acid upon light exposure (hereinafter, also referred to as “component (B)”). The component (B) includes a compound (B0) represented by General Formula (b0) (hereinafter, also referred to as “component (B0)”).

In a case where a resist film is formed using the resist composition of the present embodiment and the formed resist film is subjected to selective light exposure, an acid is generated from the component (B) at an exposed portion of the resist film, and the solubility of the component (A) in a developing solution is not changed at an unexposed portion of the resist film while the solubility of the component (A) in a developing solution is changed by the action of the acid, and thus a difference in solubility in a developing solution occurs between the exposed portion and the unexposed portion. Therefore, in a case where the resist film is developed, the exposed portion of the resist film is dissolved and removed to form a positive-tone resist pattern in a case where the resist composition is of a positive-tone, whereas the unexposed portion of the resist film is dissolved and removed to form a negative-tone resist pattern in a case where the resist composition is of a negative tone.

The resist composition of the present embodiment may be a positive-tone resist composition or a negative-tone resist composition. Further, in the formation of a resist pattern, the resist composition according to the present embodiment may be applied to an alkali developing process using an alkali developing solution in the developing treatment, or a solvent developing process using a developing solution containing an organic solvent (organic developing solution) in the developing treatment.

### <Component (A)>

In the resist composition according to the present embodiment, the component (A) contains a resin component (A1) (hereinafter, also referred to as “component (A1)”) whose solubility in a developing solution is changed by the action of an acid.

Since the polarity of the base material component before and after the light exposure is changed by using the component (A1), an excellent development contrast can be obtained not only in an alkali developing process but also in a solvent developing process.

As the component (A), another polymer compound and/or a low molecular weight compound may be used in combination with the component (A1).

In the resist composition according to the present embodiment, the component (A) may be used alone or a combination of two or more kinds thereof may be used.

### In Regard to Component (A1)

The component (A1) is a resin component whose solubility in a developing solution is changed by the action of an acid.

As the component (A1), those having a constitutional unit (a1) containing an acid decomposable group whose polarity is increased by the action of an acid are preferable.

Further, the component (A1) may have other constitutional units as necessary in addition to the constitutional unit (a1).

<<Constitutional Unit (a1)>>

The constitutional unit (a1) is a constitutional unit that contains an acid decomposable group whose polarity is increased by the action of an acid.

Examples of the acid dissociable group are the same as those which have been suggested as the acid dissociable groups of the base resin for a chemically amplified resist composition.

Specific examples of the suggested acid dissociable group of the base resin for a chemically amplified resist composition include “acetal type acid dissociable group”, “tertiary alkyl ester type acid dissociable group”, “tertiary alkyloxycarbonyl acid dissociable group”, and “secondary alkyloxycarbonyl acid dissociable group” described below.

### Acetal Type Acid Dissociable Group:

Examples of the acid dissociable group that protects a carboxy group or a hydroxyl group in the polar groups include an acid dissociable group represented by General Formula (a1-r-1) (hereinafter, also referred to as “acetal type acid dissociable group”).

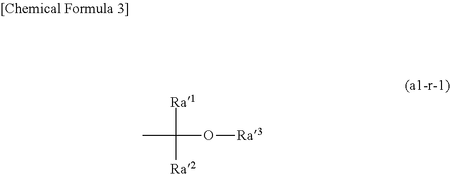

[In the formula, Ra′¹ and Ra′² represent a hydrogen atom or an alkyl group. Ra′³ represents a hydrocarbon group, and Ra′³ may be bonded to any of Ra′¹ and Ra′² to form a ring.]

In Formula (a1-r-1), it is preferable that at least one of Ra′¹ and Ra′² represents a hydrogen atom and more preferable that both Ra′¹ and Ra′² represent a hydrogen atom. In a case where Ra′¹ or Ra′² represents an alkyl group, examples of the alkyl group include the same alkyl groups described as the substituent which may be bonded to the carbon atom at the α-position in the description on α-substituted acrylic acid ester. Among these, an alkyl group having 1 to 5 carbon atoms is preferable. Specific preferred examples thereof include linear or branched alkyl groups. More specific examples thereof include a methyl group, an ethyl group, a propyl group, an isopropyl group, an n-butyl group, an isobutyl group, a tert-butyl group, a pentyl group, an isopentyl group, and a neopentyl group. Among these, a methyl group or an ethyl group is more preferable, and a methyl group is particularly preferable.

In Formula (a1-r-1), examples of the hydrocarbon group as Ra′³ include a linear or branched alkyl group and a cyclic hydrocarbon group.

The linear alkyl group has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′³ represents a cyclic hydrocarbon group, the hydrocarbon group may be an aliphatic hydrocarbon group or an aromatic hydrocarbon group and may be a polycyclic group or a monocyclic group.

As the aliphatic hydrocarbon group which is a monocyclic group, a group in which one hydrogen atom has been removed from a monocycloalkane is preferable. The number of carbon atoms in the monocycloalkane is preferably 3 to 6, and specifically, cyclopentane and cyclohexane are exemplary examples.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

In a case where the cyclic hydrocarbon group as Ra′³ becomes an aromatic hydrocarbon group, the aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group as Ra′³ include a group in which one hydrogen atom has been removed from the above-described aromatic hydrocarbon ring or aromatic heterocyclic ring (such as an aryl group or a heteroaryl group); a group in which one hydrogen atom has been removed from an aromatic compound having two or more aromatic rings (such as biphenyl or fluorene); and a group in which one hydrogen atom of the above-described aromatic hydrocarbon ring or aromatic heterocyclic ring has been substituted with an alkylene group (for example, an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aromatic hydrocarbon ring or aromatic heterocyclic ring is preferably in a range of 1 to 4, more preferably 1 or 2, and particularly preferably 1.

The cyclic hydrocarbon group as Ra′³ may include a substituent. Examples of this substituent include Ra^{x5} described above.

In a case where Ra′³ is bonded to any of Ra′¹ and Ra′² to form a ring, the cyclic group is preferably a 4- to 7-membered ring and more preferably a 4- to 6-membered ring. Specific examples of the cyclic group include a tetrahydropyranyl group and a tetrahydrofuranyl group.

Tertiary alkyl ester type acid dissociable group:

Examples of the acid dissociable group that protects a carboxy group among the polar groups include an acid dissociable group represented by General Formula (a1-r-2).

Among examples of the acid dissociable group represented by Formula (a1-r-2), a group formed of an alkyl group is referred to as “tertiary alkyl ester type acid dissociable group” for convenience.

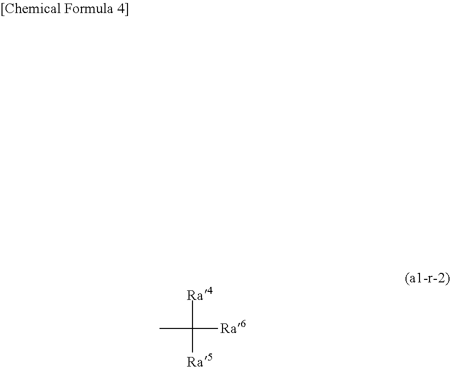

[In the formula, Ra′⁴ to Ra′⁶ each independently represent a hydrocarbon group, and Ra′⁵ and Ra′⁶ may be bonded to each other to form a ring.]

Examples of the hydrocarbon group as Ra′⁴ include a linear or branched alkyl group, a chain-like or cyclic alkenyl group, and a cyclic hydrocarbon group.

Examples of the linear or branched alkyl group and the cyclic hydrocarbon group (an aliphatic hydrocarbon group which is a monocyclic group, an aliphatic hydrocarbon group which is a polycyclic group, or an aromatic hydrocarbon group) as Ra′⁴ include the same groups as those for Ra′³.

As the chain-like or cyclic alkenyl group as Ra′⁴, an alkenyl group having 2 to 10 carbon atoms is preferable.

Examples of the hydrocarbon group as Ra′⁵ or Ra′⁶ include the same groups as those for Ra′³.

In a case where Ra′⁵ and Ra′⁶ are bonded to each other to form a ring, suitable examples thereof include a group represented by General Formula (a1-r2-1), a group represented by General Formula (a1-r2-2), and a group represented by General Formula (a1-r2-3).

Meanwhile, in a case where Ra′⁴ to Ra′⁶ represent an independent hydrocarbon group without being bonded to one another, suitable examples thereof include a group represented by General Formula (a1-r2-4).

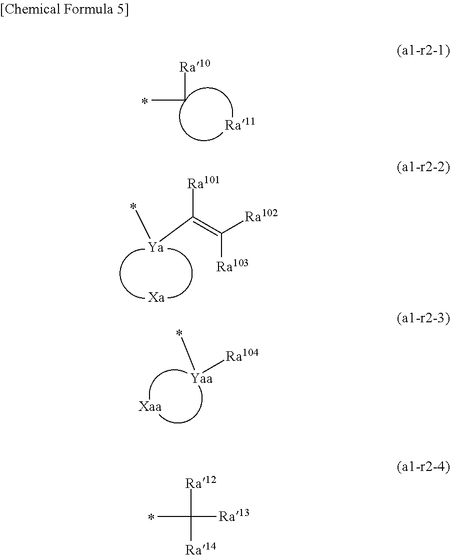

[In Formula (a1-r2-1), Ra′¹⁰ represents a linear or branched alkyl group having 1 to 12 carbon atoms, in which a part thereof may be substituted with a halogen atom or a heteroatom-containing group. Ra′¹¹ represents a group that forms an aliphatic cyclic group with the carbon atom to which Ra′¹⁰ has been bonded. In Formula (a1-r2-2), Ya represents a carbon atom. Xa represents a group that forms a cyclic hydrocarbon group with Ya. Some or all hydrogen atoms in this cyclic hydrocarbon group may be substituted. Ra¹⁰¹ to Ra¹⁰³ each independently represent a hydrogen atom, a chain-like monovalent saturated hydrocarbon group having 1 to 10 carbon atoms, or a monovalent aliphatic cyclic saturated hydrocarbon group having 3 to 20 carbon atoms. Some or all hydrogen atoms in the chain-like saturated hydrocarbon group and the aliphatic cyclic saturated hydrocarbon group may be substituted. Two or more of Ra¹⁰¹ to Ra¹⁰³ may be bonded to one another to form a cyclic structure. In Formula (a1-r2-3), Yaa represents a carbon atom. Xaa represents a group that forms an aliphatic cyclic group with Yaa. Ra¹⁰⁴ represents an aromatic hydrocarbon group which may have a substituent. In Formula (a1-r2-4), Ra′¹² and Ra′¹³ each independently represent a monovalent chain-like saturated hydrocarbon group having 1 to 10 carbon atoms. Some or all of hydrogen atoms contained in the chain-like saturated hydrocarbon group may be substituted. Ra′¹⁴ represents a hydrocarbon group which may have a substituent. * represents a bonding site (the same applies hereinafter).]

In Formula (a1-r2-1), Ra′¹⁰ represents a linear or branched alkyl group having 1 to 12 carbon atoms, in which a part thereof may be substituted with a halogen atom or a heteroatom-containing group.

The linear alkyl group as Ra′¹⁰ has 1 to 12 carbon atoms, preferably 1 to 10 carbon atoms, and particularly preferably 1 to 5 carbon atoms.

Examples of the branched alkyl group as Ra′¹⁰ include those for Ra′³ described above.

The alkyl group in Ra′¹⁰ may be partially substituted with a halogen atom or a heteroatom-containing group. For example, some hydrogen atoms constituting the alkyl group may be substituted with a halogen atom or a heteroatom-containing group. Further, some carbon atoms (methylene group or the like) constituting the alkyl group may be substituted with a heteroatom-containing group.

Examples of the heteroatoms here include an oxygen atom, a nitrogen atom, and a sulfur atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), preferred examples of Ra′¹¹ (an aliphatic cyclic group that is formed together with a carbon atom to which Ra′¹⁰ is bonded) include the groups described as the aliphatic hydrocarbon group (alicyclic hydrocarbon group) which is a monocyclic group or a polycyclic group as Ra′³ in Formula (a1-r-1). Among these, it is preferably a monocyclic alicyclic hydrocarbon group, and specifically, it is more preferably a cyclopentyl group or a cyclohexyl group.

In Formula (a1-r2-2), examples of the cyclic hydrocarbon group that is formed by Xa together with Ya include a group formed by further removing one or more hydrogen atoms from the cyclic monovalent hydrocarbon group (an aliphatic hydrocarbon group) as Ra′³ in Formula (a1-r-1).

The cyclic hydrocarbon group that is formed by Xa together with Ya may have a substituent. Examples of the substituent include those which are the same as the substituents which may be included in the cyclic hydrocarbon group as Ra′³.

In Formula (a1-r2-2), examples of the chain-like monovalent saturated hydrocarbon group having 1 to 10 carbon atoms as Ra¹⁰¹ to Ra¹⁰³ include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms as Ra¹⁰¹ to Ra¹⁰³ include a monocyclic aliphatic saturated hydrocarbon group such as a cyclopropyl group, a cyclobutyl group, a cyclopentyl group, a cyclohexyl group, a cycloheptyl group, a cyclooctyl group, a cyclodecyl group, or a cyclododecyl group; and a polycyclic aliphatic saturated hydrocarbon group such as a bicyclo[2.2.2]octanyl group, a tricyclo[5.2.1.0^{2,6}]decanyl group, a tricyclo[3.3.1.1^{3,7}] decanyl group, a tetracyclo[6.2.1.1^{3,6}.0^{2,7}]dodecanyl group, or an adamantyl group.

From the viewpoint of ease of synthesis, Ra¹⁰¹ to Ra¹⁰³ represent preferably a hydrogen atom or a chain-like monovalent saturated hydrocarbon group having 1 to 10 carbon atoms, more preferably a hydrogen atom, a methyl group, or an ethyl group, and particularly preferably a hydrogen atom.

Examples of the substituent included in the chain-like saturated hydrocarbon group or the aliphatic cyclic saturated hydrocarbon group represented by Ra¹⁰¹ to Ra¹⁰³ are the same as those for Ra^{x5}.

Examples of the group having a carbon-carbon double bond generated by two or more of Ra¹⁰¹ to Ra¹⁰³ being bonded to one another to form a cyclic structure include a cyclopentenyl group, a cyclohexenyl group, a methylcyclopentenyl group, a methylcyclohexenyl group, a cyclopentylidenethenyl group, and a cyclohexylidenethenyl group. Among these, from the viewpoint of ease of synthesis, a cyclopentenyl group, a cyclohexenyl group, or a cyclopentylidenethenyl group is preferable.

In Formula (a1-r2-3), preferred examples of the aliphatic cyclic group that is formed by Xaa together with Yaa include the group described as the aliphatic hydrocarbon group which is a monocyclic group or a polycyclic group as Ra′³ in Formula (a1-r-1).

In Formula (a1-r2-3), examples of the aromatic hydrocarbon group as Ra¹⁰⁴ include a group in which one or more hydrogen atoms have been removed from an aromatic hydrocarbon ring having 5 to 30 carbon atoms. Among the examples, Ra¹⁰⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from benzene or naphthalene, and most preferably a group in which one or more hydrogen atoms have been removed from benzene.

Examples of the substituent that Ra¹⁰⁴ in Formula (a1-r2-3) may have include a methyl group, an ethyl group, propyl group, a hydroxy group, a carboxy group, a halogen atom, an alkoxy group (a methoxy group, an ethoxy group, a propoxy group, a butoxy group, and the like), and an alkyloxycarbonyl group.

In Formula (a1-r2-4), Ra′¹² and Ra′¹³ each independently represent a monovalent chain-like saturated hydrocarbon group having 1 to 10 carbon atoms. Examples of the monovalent chain-like saturated hydrocarbon group having 1 to 10 carbon atoms as Ra′¹² and Ra′¹³ include the same one as the monovalent chain-like saturated hydrocarbon group having 1 to 10 carbon atoms as Ra¹⁰¹ to Ra¹⁰³ as described above. Some or all of hydrogen atoms contained in the chain-like saturated hydrocarbon group may be substituted.

Ra′¹² and Ra′¹³ represent preferably an alkyl group having 1 to 5 carbon atoms, more preferably an alkyl group having 1 to 5 carbon atoms, still more preferably a methyl group or an ethyl group, and particularly preferably a methyl group.

In a case where the chain-like saturated hydrocarbon group represented by Ra′¹² and Ra′¹³ is substituted, examples of the substituent are those for Ra^{x5} described above.

In Formula (a1-r2-4), Ra′¹⁴ represents a hydrocarbon group which may have a substituent. Examples of the hydrocarbon group as Ra′¹⁴ include a linear or branched alkyl group and a cyclic hydrocarbon group.

The linear alkyl group as Ra′¹⁴ has preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group, or an n-butyl group is preferable, and a methyl group or an ethyl group is more preferable.

The branched alkyl group as Ra′¹⁴ has preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. Among these, an isopropyl group is preferable.

In a case where Ra′¹⁴ represents a cyclic hydrocarbon group, the hydrocarbon group may be an aliphatic hydrocarbon group or an aromatic hydrocarbon group and may be polycyclic or monocyclic.

As the aliphatic hydrocarbon group which is a monocyclic group, a group in which one hydrogen atom has been removed from a monocycloalkane is preferable. The number of carbon atoms in the monocycloalkane is preferably 3 to 6, and specifically, cyclopentane and cyclohexane are exemplary examples.

As the aliphatic hydrocarbon group which is a polycyclic group, a group in which one hydrogen atom has been removed from a polycycloalkane is preferable. As the polycycloalkane, a group having 7 to 12 carbon atoms is preferable, and specific examples thereof include adamantane, norbornane, isobornane, tricyclo[5.2.1.0^{2,6}]decane, and tetracyclododecane.

Examples of the aromatic hydrocarbon group as Ra′¹⁴ include the same groups as those for the aromatic hydrocarbon group as Ra¹⁰⁴. Among these, Ra′¹⁴ represents preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene, or phenanthrene, still more preferably a group in which one or more hydrogen atoms have been removed from benzene, naphthalene, or anthracene, particularly preferably a group in which one or more hydrogen atoms have been removed from naphthalene or anthracene, and most preferably a group in which one or more hydrogen atoms have been removed from naphthalene.

Examples of the substituent which may be included in Ra′¹⁴ include the same groups as those for the substituent which may be included in Ra¹⁰⁴.

In a case where Ra′¹⁴ in Formula (a1-r2-4) represents a naphthyl group, the position bonded to the tertiary carbon atom in Formula (a1-r2-4) may be the 1-position or the 2-position of the naphthyl group.

In a case where Ra′¹⁴ in Formula (a1-r2-4) represents an anthryl group, the position bonded to the tertiary carbon atom in Formula (a1-r2-4) may be the 1-position, the 2-position, or the 9-position of the anthryl group.

Specific examples of the group represented by Formula (a1-r2-1) are shown below.

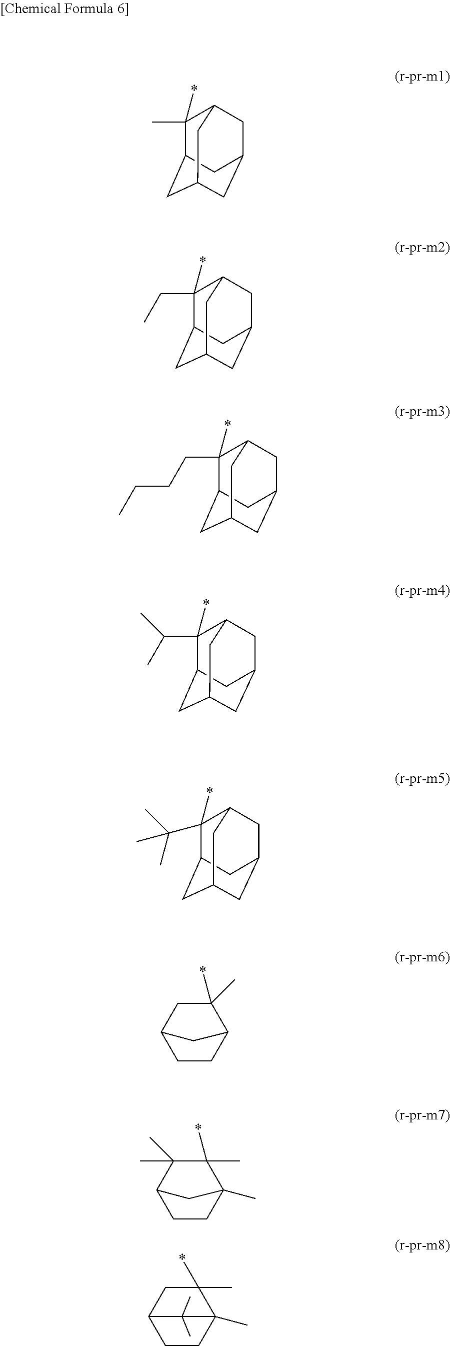

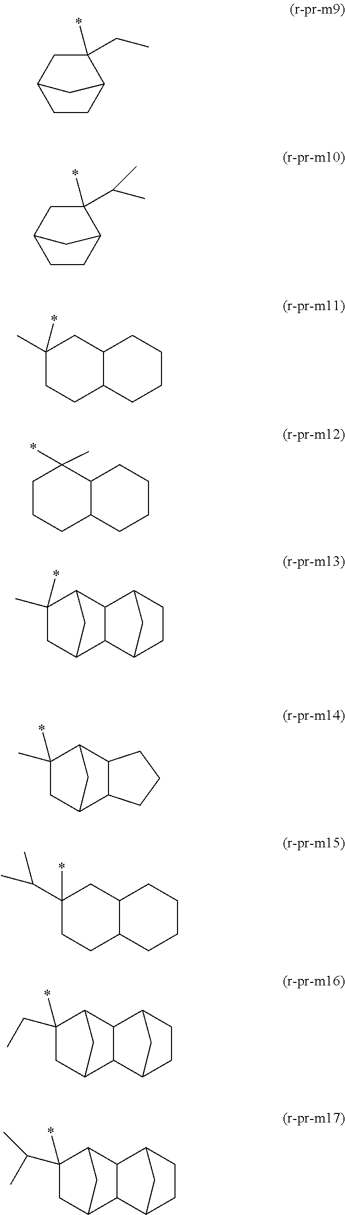

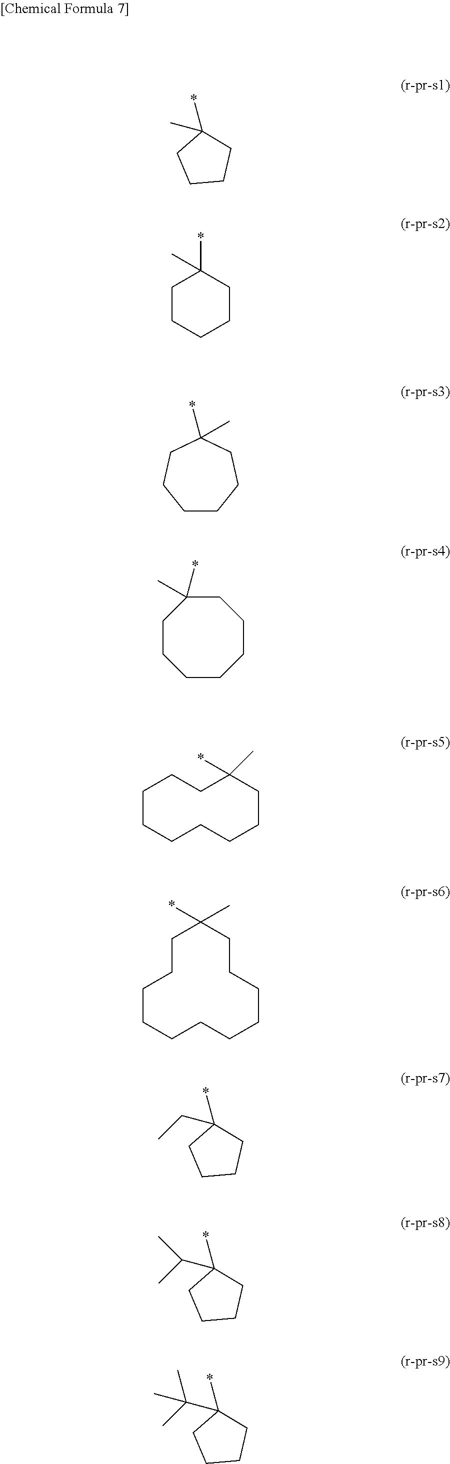

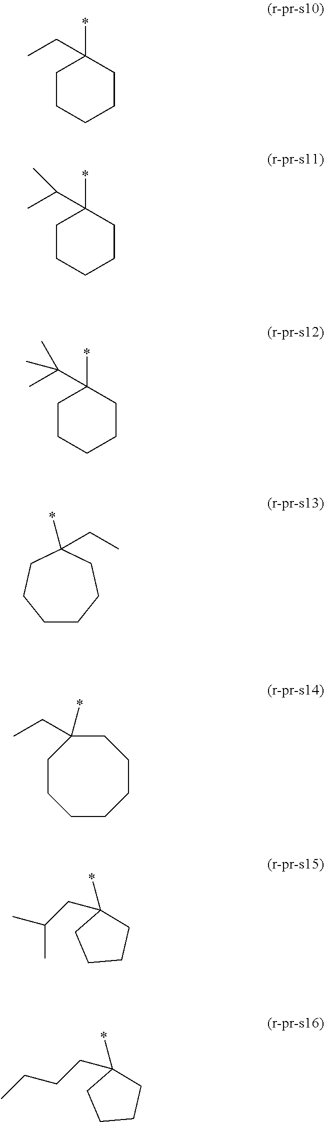

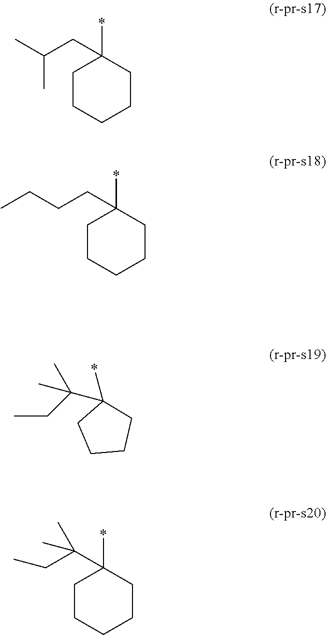 [SMILES: *C1(CC(C)C)CCCCC1 ; *C1(CCCC)CCCCC1 ; *C1(C(C)(C)CC)CCCC1 ; *C1(C(C)(C)CC)CCCCC1]

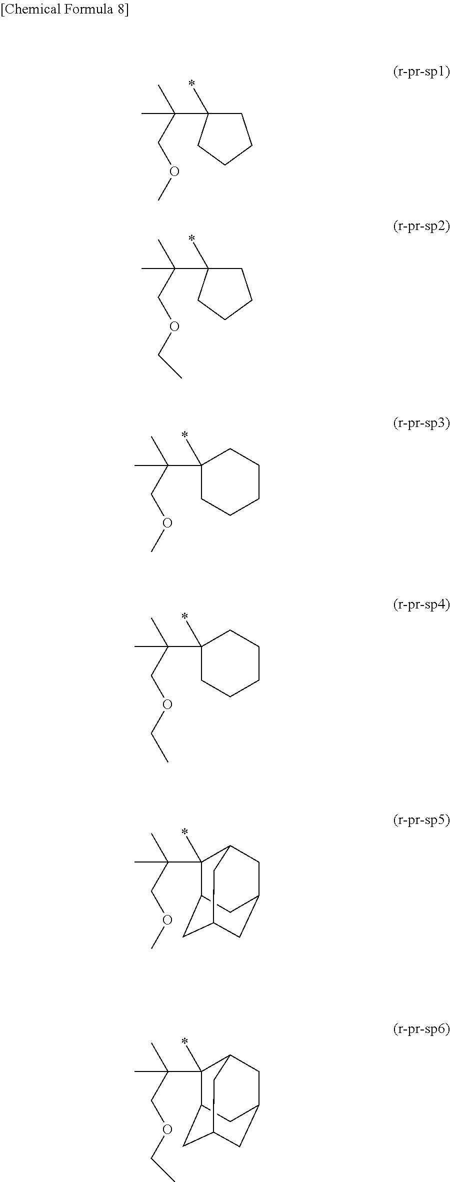

Specific examples of the group represented by Formula (a1-r2-2) are shown below.

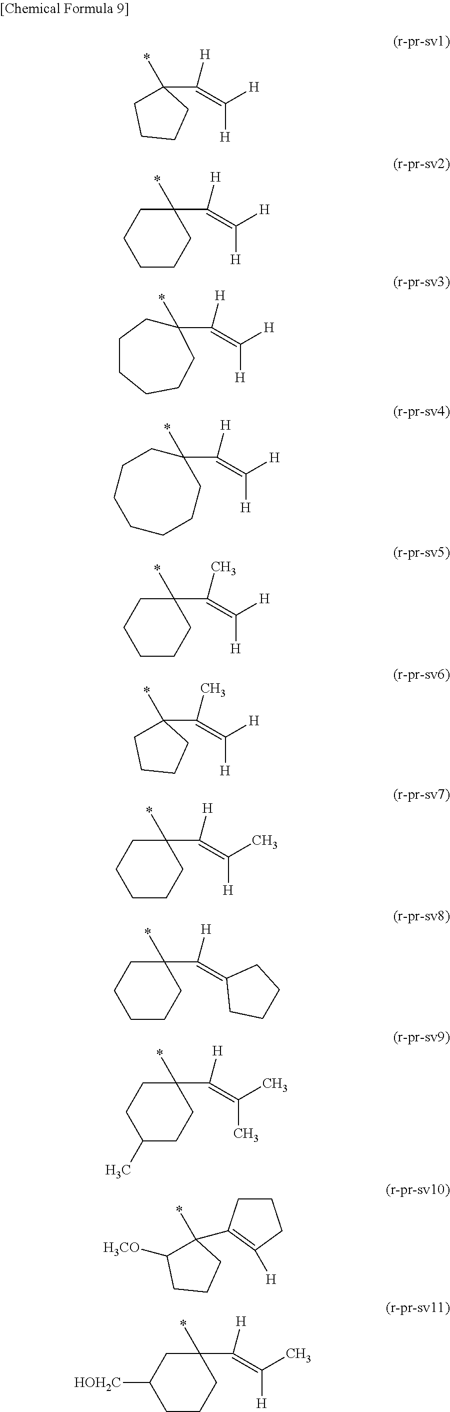

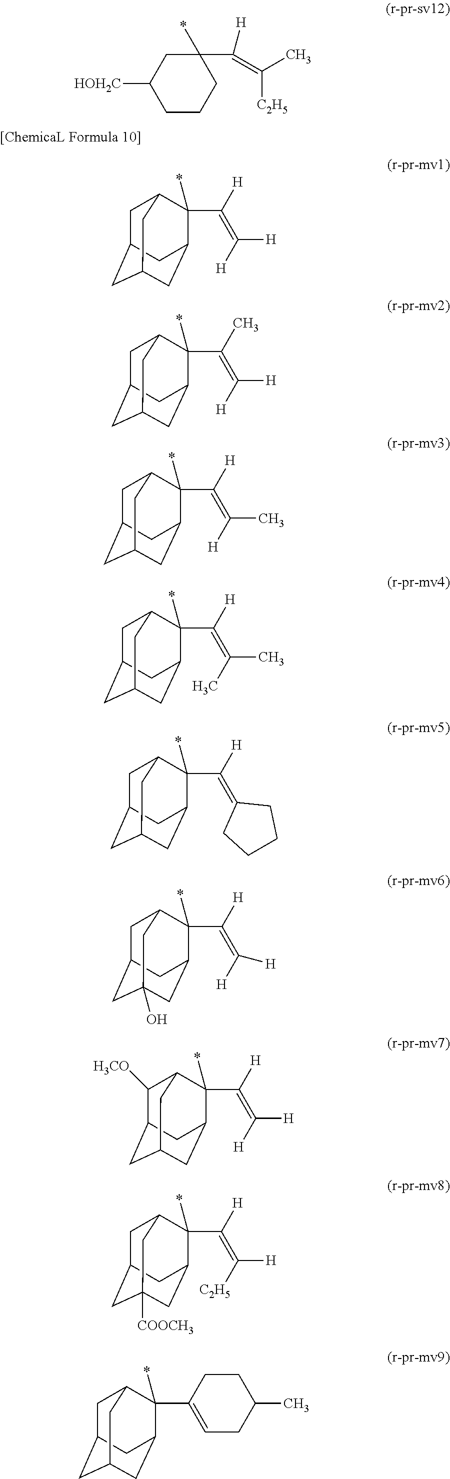

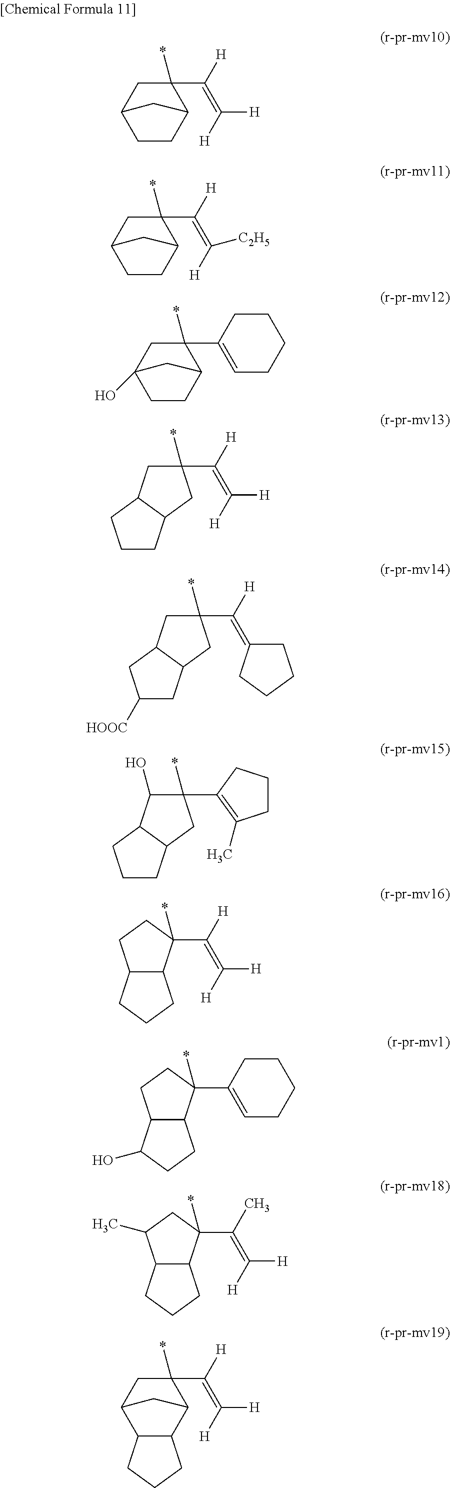

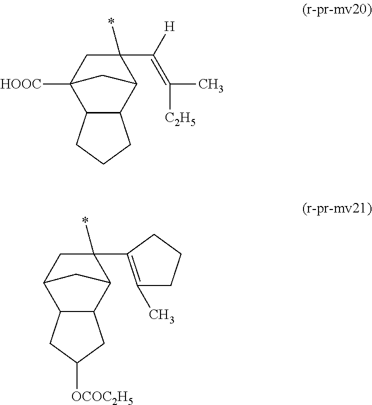

Specific examples of the group represented by Formula (a1-r2-3) are shown below.

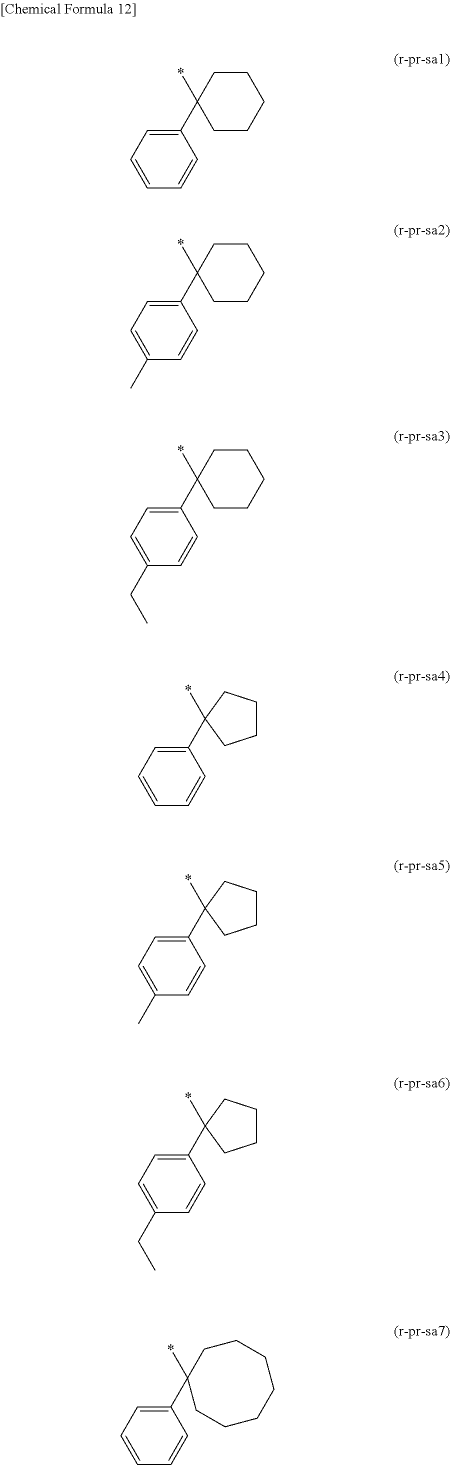

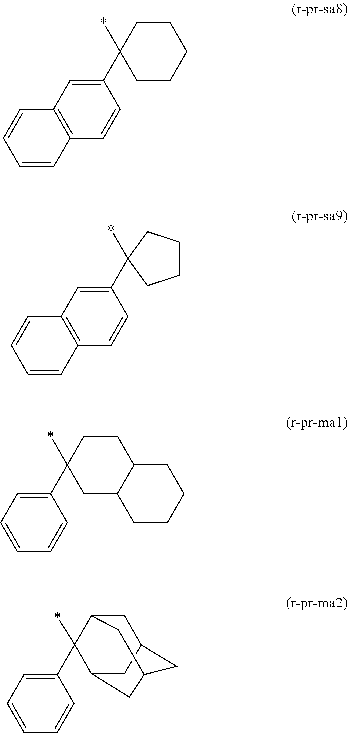 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by Formula (a1-r2-4) are shown below.

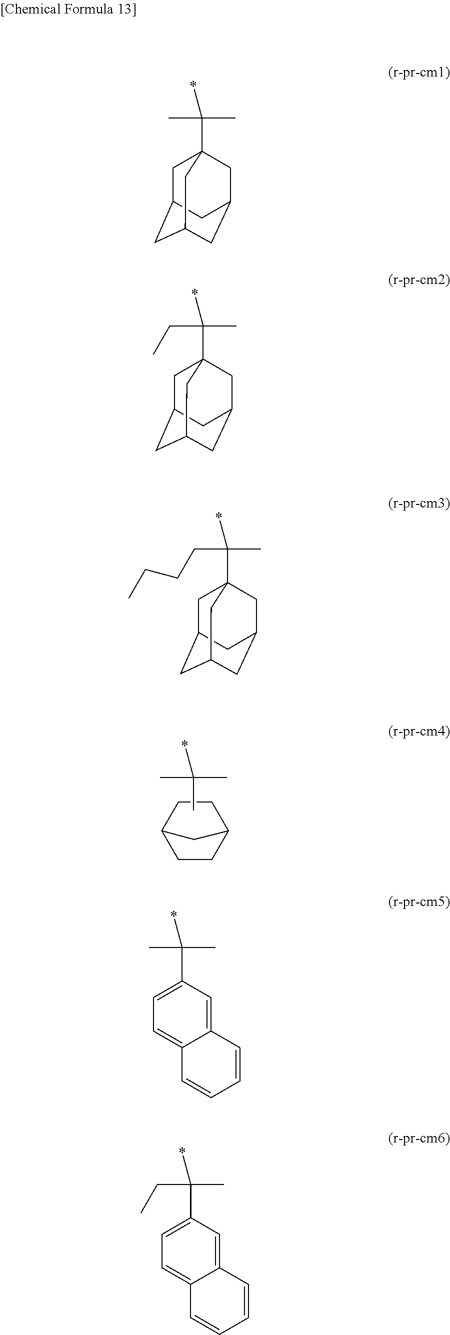

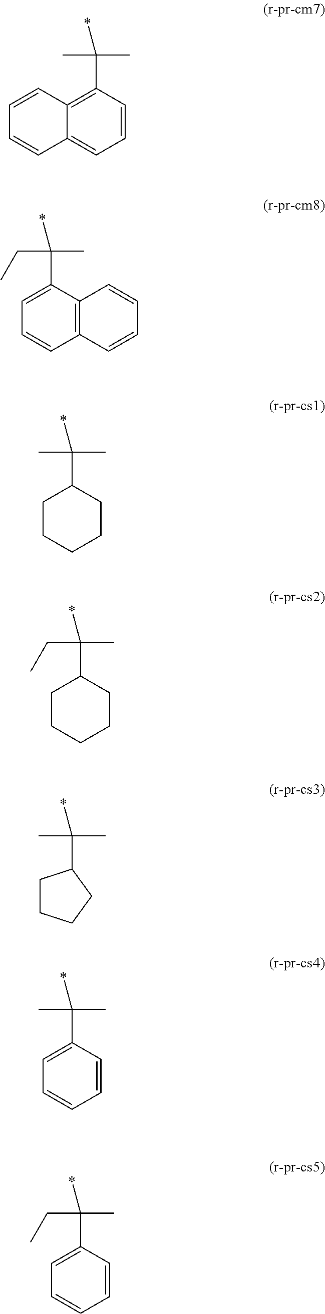

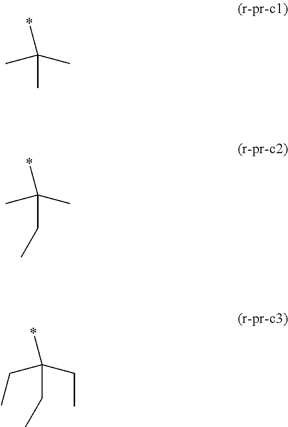

Tertiary alkyloxycarbonyl acid dissociable group:

Examples of the acid dissociable group that protects a hydroxyl group among the polar groups include an acid dissociable group (hereinafter, also referred to as “tertiary alkyloxycarbonyl acid dissociable group” for convenience) represented by General Formula (a1-r-3).

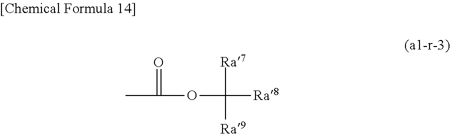

[In the formula, Ra′⁷ to Ra′⁹ each independently represent an alkyl group.]

In Formula (a1-r-3), Ra′⁷ to Ra′⁹ each represent preferably an alkyl group having 1 to 5 carbon atoms and more preferably an alkyl group having 1 to 3 carbon atoms.

Further, the total number of carbon atoms in each alkyl group is preferably in a range of 3 to 7, more preferably in a range of 3 to 5, and most preferably 3 or 4.

Secondary alkyl ester type acid dissociable group:

Examples of the acid dissociable group that protects a carboxy group among the polar groups include an acid dissociable group represented by General Formula (a1-r-4).

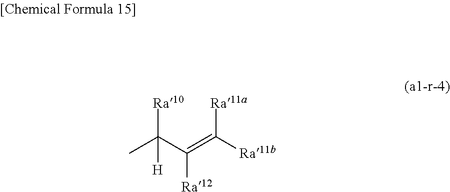

[In the formula, Ra′¹⁰ represents a hydrocarbon group. Ra′^{11a} and Ra′^{11b} each independently represent a hydrogen atom, a halogen atom, or an alkyl group. Ra′¹² represents a hydrogen atom or a hydrocarbon group. Ra′¹⁰ and Ra′^{11a} or Ra′^{11b} may be bonded to each other to form a ring. Ra′^{11a} or Ra′^{11b} and Ra′¹² may be bonded to each other to form a ring.]

Examples of the hydrocarbon group as Ra′¹⁰ or Ra′¹² in the formula include the same groups as those for Ra′³.

Examples of the alkyl group as Ra′^{11a} and Ra′^{11b} in the formula include the same groups as those for the alkyl group as Ra′¹.

In the formula, the hydrocarbon group as Ra′¹⁰ or Ra′¹² and the alkyl group as Ra′^{11a} and Ra′^{11b} may have a substituent. Examples of this substituent include Ra^{x5} described above.

Ra′¹⁰ and Ra′^{11a} or Ra′^{11b} may be bonded to each other to form a ring. The ring may be a polycyclic ring or a monocyclic ring, and may be an alicyclic ring or an aromatic ring.

The alicyclic ring and the aromatic ring may have a heteroatom.

Among the examples described above, as the ring formed by Ra′¹⁰ and Ra′^{11a} or Ra′^{11b} being bonded to each other, monocycloalkene, a ring in which some carbon atoms of monocycloalkene are substituted with heteroatoms (such as an oxygen atom and a sulfur atom), or monocycloalkadiene is preferable, cycloalkene having 3 to 6 carbon atoms is preferable, and cyclopentene or cyclohexene is preferable.

The ring formed by Ra′¹⁰ and Ra′^{11a} or Ra′^{11b} being bonded to each other may be a condensed ring. Specific examples of the condensed ring include indane.

The ring formed by Ra′¹⁰ and Ra′^{11a} or Ra′^{11b} being bonded to each other may have a substituent. Examples of this substituent include Ra^{x5} described above.

Ra′^{11a} or Ra′^{11b} and Ra′¹² may be bonded to each other to form a ring, and examples of the ring include the rings formed by Ra′¹⁰ and Ra′^{11a} or Ra′^{11b} being bonded to each other.

Specific examples of the group represented by Formula (a1-r-4) are shown below.

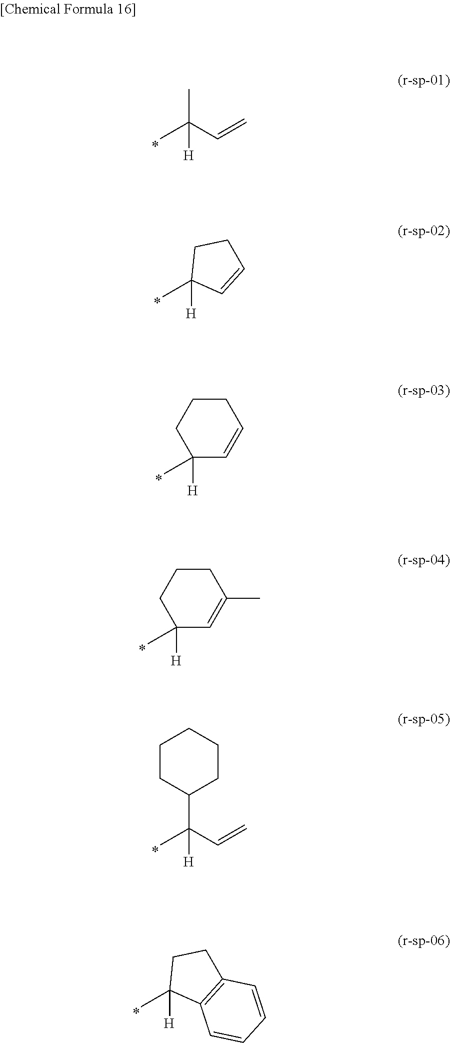

Examples of the constitutional unit (a1) include a constitutional unit derived from acrylic acid ester in which the hydrogen atom bonded to the carbon atom at the α-position may be substituted with a substituent; a constitutional unit derived from acrylamide; a constitutional unit in which at least some hydrogen atoms in a hydroxyl group of a constitutional unit derived from hydroxystyrene or a hydroxystyrene derivative are protected by a substituent containing the acid decomposable group; and a constitutional unit in which at least some hydrogen atoms in-C(═O)—OH of a constitutional unit derived from vinylbenzoic acid or a vinylbenzoic acid derivative are protected by a substituent containing the acid decomposable group.

Among the examples, as the constitutional unit (a1), a constitutional unit derived from acrylic acid ester in which the hydrogen atom bonded to the carbon atom at the α-position may be substituted with a substituent is preferable.

Specific preferred examples of such a constitutional unit (a1) include constitutional units represented by General Formula (a1-1) or (a1-2) shown below.

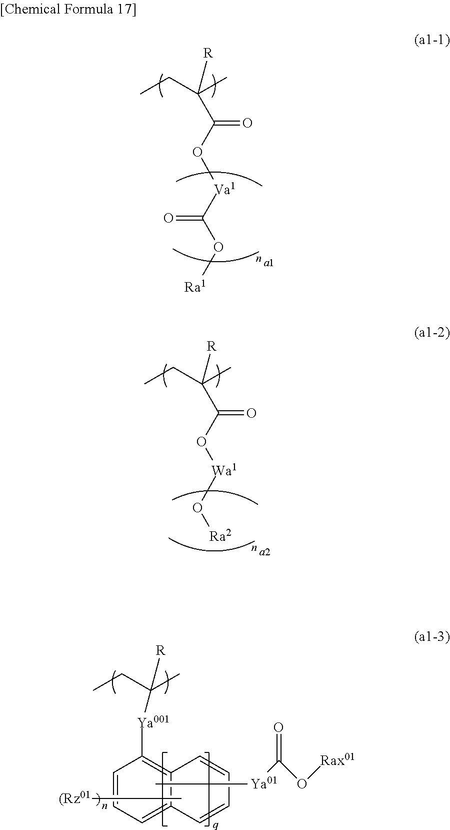

[In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ represents an integer of 0 to 2. Ra¹ represents an acid dissociable group represented by General Formula (a1-r-1), (a1-r-2), or (a1-r-4). Wa¹ represents a (nₐ₂+1)-valent hydrocarbon group, nₐ₂ represents an integer of 1 to 3, and Ra² represents an acid dissociable group represented by General Formula (a1-r-1) or (a1-r-3). Ya⁰⁰¹ represents a single bond or a divalent linking group. Ya⁰¹ represents a single bond or a divalent linking group. Rax⁰¹ represents an acid dissociable group represented by General Formula (a1-r-1), (a1-r-2), or (a1-r-4). Rz⁰¹ represents an alkyl group, a halogen atom, a halogenated alkyl group, a hydroxy group, or an alkoxy group. q represents an integer of 0 to 3. n represents an integer of 0 or greater. Here, n≤q×2+4 is satisfied.]

In General Formulae (a1-1) to (a1-3), R represents a hydrogen atom, an alkyl group having 1 to 5 carbon atoms, or a halogenated alkyl group having 1 to 5 carbon atoms. The alkyl group having 1 to 5 carbon atoms as R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all hydrogen atoms in the alkyl group having 1 to 5 carbon atoms have been substituted with halogen atoms. As the halogen atom, a fluorine atom is particularly preferable.

R represents preferably a hydrogen atom, an alkyl group having 1 to 5 carbon atoms, or a fluorinated alkyl group having 1 to 5 carbon atoms, and most preferably a hydrogen atom or a methyl group from the viewpoint of industrial availability.

In Formula (a1-1), the divalent hydrocarbon group as Va¹ may be an aliphatic hydrocarbon group or an aromatic hydrocarbon group.

The aliphatic hydrocarbon group as the divalent hydrocarbon group represented by Va¹ may be saturated or unsaturated. In general, it is preferable that the aliphatic hydrocarbon group is saturated.

More specific examples of the aliphatic hydrocarbon group include a linear or branched aliphatic hydrocarbon group and an aliphatic hydrocarbon group having a ring in the structure thereof.

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group has preferably 2 to 10 carbon atoms, more preferably 3 to 6 carbon atoms, still more preferably 3 or 4 carbon atoms, and most preferably 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃) (CH₂CH₃)—, —C(CH₃) (CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Examples of the aliphatic hydrocarbon group having a ring in the structure thereof include an alicyclic hydrocarbon group (a group in which two hydrogen atoms have been removed from an aliphatic hydrocarbon ring), a group in which the alicyclic hydrocarbon group is bonded to the terminal of the linear or branched aliphatic hydrocarbon group, and a group in which the alicyclic hydrocarbon group is interposed in the middle of the linear or branched aliphatic hydrocarbon group. Examples of the linear or branched aliphatic hydrocarbon group include the same groups as those for the linear aliphatic hydrocarbon group or the branched aliphatic hydrocarbon group.

The alicyclic hydrocarbon group has preferably 3 to 20 carbon atoms and more preferably 3 to 12 carbon atoms.

The alicyclic hydrocarbon group may be monocyclic or polycyclic. The monocyclic alicyclic hydrocarbon group is preferably a group obtained by removing two hydrogen atoms from a monocycloalkane. The monocycloalkane has preferably 3 to 6 carbon atoms, and specific examples thereof include cyclopentane and cyclohexane. As the polycyclic alicyclic hydrocarbon group, a group in which two hydrogen atoms have been removed from a polycycloalkane is preferable. The number of carbon atoms in the polycycloalkane is preferably in a range of 7 to 12, and specific examples thereof include adamantane, norbornane, isobornane, tricyclo[5.2.1.0^{2,6}]decane, and tetracyclododecane.

The aromatic hydrocarbon group as the divalent hydrocarbon group represented by Va¹ is a hydrocarbon group having an aromatic ring.

The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 12 carbon atoms. Here, the number of carbon atoms in a substituent is not included in the number of carbon atoms.

Specific examples of the aromatic ring contained in the aromatic hydrocarbon group include aromatic hydrocarbon rings such as benzene, biphenyl, fluorene, naphthalene, anthracene, and phenanthrene; and aromatic heterocyclic rings in which some carbon atoms constituting the above-described aromatic hydrocarbon rings have been substituted with heteroatoms. Examples of the heteroatom in the aromatic heterocyclic rings include an oxygen atom, a sulfur atom, and a nitrogen atom.

Specific examples of the aromatic hydrocarbon group include a group in which two hydrogen atoms have been removed from the above-described aromatic hydrocarbon ring (an arylene group); and a group in which one hydrogen atom of a group (an aryl group) formed by removing one hydrogen atom from the aromatic hydrocarbon ring has been substituted with an alkylene group (for example, a group formed by further removing one more hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

In Formula (a1-1), Ra¹ represents preferably an acid dissociable group represented by General Formula (a1-r-2) or (a1-r-4) and, among these, more preferably a group represented by General Formula (a1-r2-1) or an acid dissociable group represented by General Formula (a1-r-4).

In Formula (a1-2), the (nₐ₂+1)-valent hydrocarbon group as Wa¹ may be an aliphatic hydrocarbon group or an aromatic hydrocarbon group. The aliphatic hydrocarbon group indicates a hydrocarbon group that has no aromaticity and may be saturated or unsaturated. In general, it is preferable that the aliphatic hydrocarbon group is saturated. Examples of the aliphatic hydrocarbon group include a linear or branched aliphatic hydrocarbon group, an aliphatic hydrocarbon group having a ring in the structure thereof, and a group obtained by combining the linear or branched aliphatic hydrocarbon group and the aliphatic hydrocarbon group having a ring in the structure thereof.

The valency of nₐ₂+1 is preferably divalent, trivalent, or tetravalent and more preferably divalent or trivalent.

In Formula (a1-2), it is preferable that Ra² represents an acid dissociable group represented by General Formula (a1-r-1).

In Formula (a1-3), the divalent linking group as Ya⁰⁰¹ is not particularly limited, and suitable examples thereof include a divalent hydrocarbon group which may have a substituent and a divalent linking group having a heteroatom.

Among these, it is preferable that Ya⁰⁰¹ represents preferably an ester bond [—C(═O)—O— or —O—C(═O)—], an ether bond (—O—), a linear or branched alkylene group, an aromatic hydrocarbon group, a combination thereof, or a single bond. The alkylene group has preferably 1 to 10 carbon atoms, more preferably 1 to 6 carbon atoms, still more preferably 1 to 4 carbon atoms, and particularly preferably 1 to 3 carbon atoms.

Among these, Ya⁰⁰¹ represents more preferably a combination of an ester bond [—C(═O)—O— or —O—C(═O)—] and a linear alkylene group, or a single bond and still more preferably a single bond.

In Formula (a1-3), the divalent linking group as Ya⁰¹ is not particularly limited, and suitable examples thereof include a divalent hydrocarbon group which may have a substituent and a divalent linking group having a heteroatom.

Among these, it is preferable that Ya⁰¹ represents an ester bond [—C(═O)—O— or —O—C(═O)—], an ether bond (—O—), a linear or branched alkylene group, an aromatic hydrocarbon group, a combination thereof, or a single bond. Among these, Ya⁰¹ is more preferably a combination of an ester bond [—C(═O)—O— or —O—C(═O)—] and a linear alkylene group, or a single bond, and still more preferably a single bond.

In Formula (a1-3), Rax⁰¹ represents preferably an acid dissociable group represented by General Formula (a1-r-2) or (a1-r-4) and, among these, more preferably an acid dissociable group represented by General Formula (a1-r-2) and still more preferably a group represented by General Formula (a1-r2-1).

In Formula (a1-3), the alkyl group, the halogenated alkyl group, and the alkoxy group as Rz⁰¹ have preferably 1 to 10 carbon atoms, more preferably 1 to 5 carbon atoms, still more preferably 1 to 3 carbon atoms, and particularly preferably 1 or 2 carbon atoms. The alkyl group, the halogenated alkyl group, and the alkoxy group may be linear or branched.

As the halogen atom represented by Rz⁰¹, an iodine atom is preferable. As the halogen atom of the halogenated alkyl group represented by Rz⁰¹, a fluorine atom, an iodine atom, or a bromine atom is preferable, and a fluorine atom is more preferable.

Rz⁰¹ represents preferably an alkoxy group or a hydroxy group and more preferably a hydroxy group.

In General Formula (a1-3), q represents an integer of 0 to 3. A benzene structure is formed in a case where q represents 0, a naphthalene structure is formed in a case where q represents 1, an anthracene structure is formed in a case where q represents 2, and a tetracene structure is formed in a case where q represents 3.

In General Formula (a1-3), n represents an integer of 0 or greater, preferably 1 to 5, more preferably 0 to 3, and still more preferably 1 or 2. In a case where n represents an integer of 2 or greater, two or more Rz⁰¹'s may be the same as or different from each other.

In Formula (a1-3), n≤q×2+4 is satisfied. For example, in a case where q represents 1 and thus a naphthalene structure is formed, all six hydrogen atoms of the naphthalene may be substituted with hydroxy groups. In addition, the substitution positions of Ya⁰⁰¹, the -Ya⁰¹-C(═O)—O—Ra⁰¹ group, and the hydroxy group in the naphthalene are not particularly limited.

Specific examples of the constitutional unit (a1) are shown below. In the formulae shown below, R^{α} represents a hydrogen atom, a methyl group, or a trifluoromethyl group.

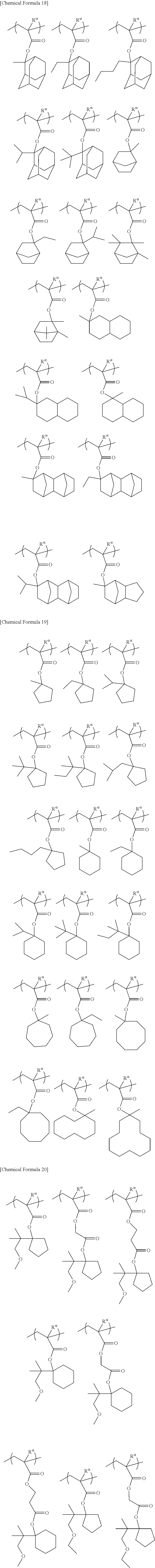

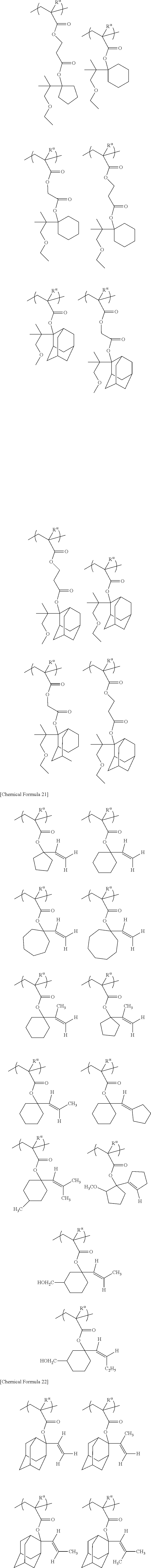

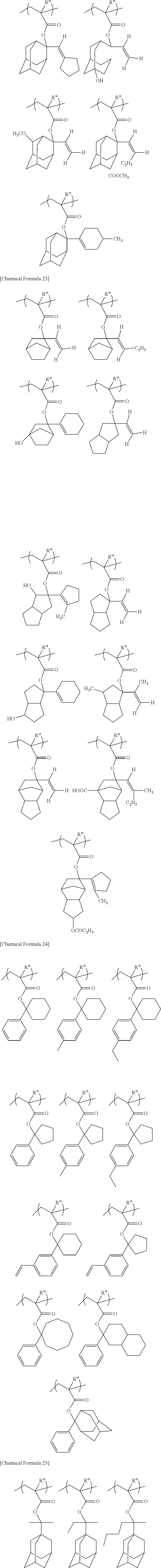

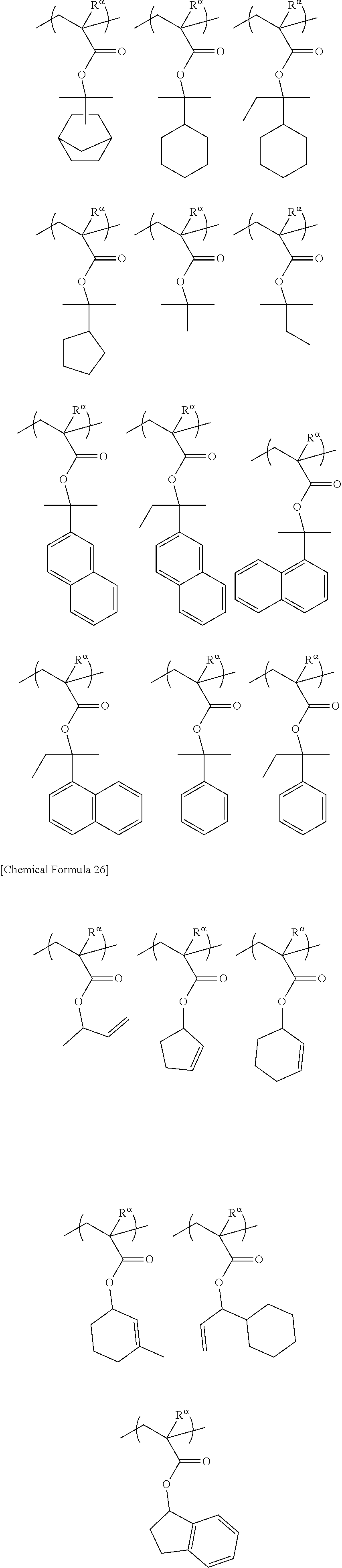

In the formulae shown below, Ra represents a hydrogen atom, a methyl group, or a trifluoromethyl group. Rz represents a hydrogen atom, an alkyl group, a halogen atom, a halogenated alkyl group, a hydroxy group, or an alkoxy group.

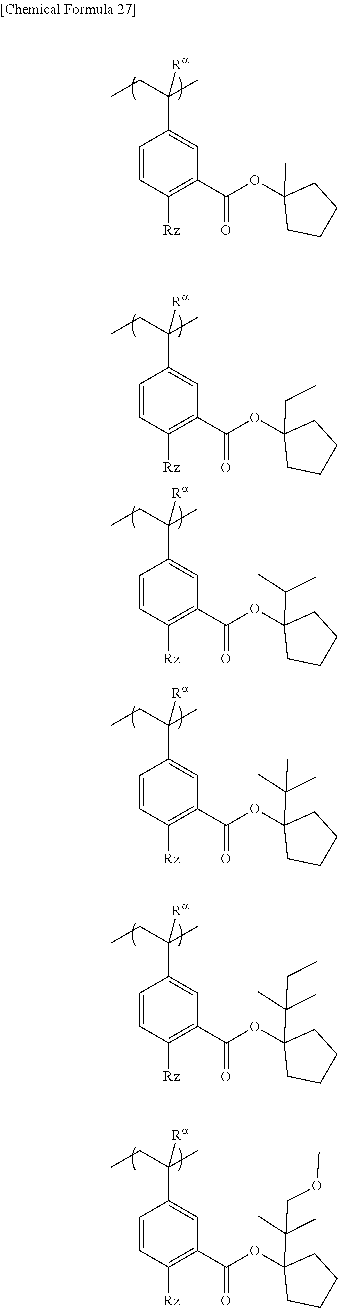

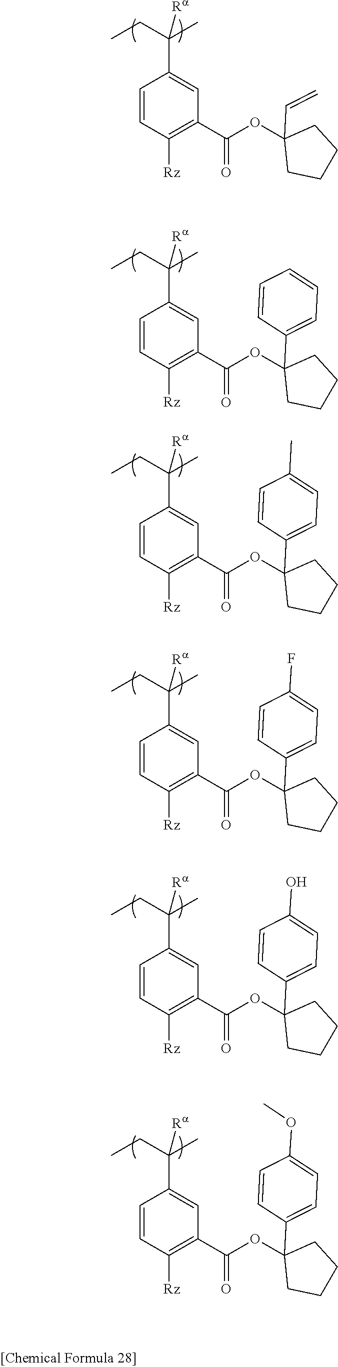

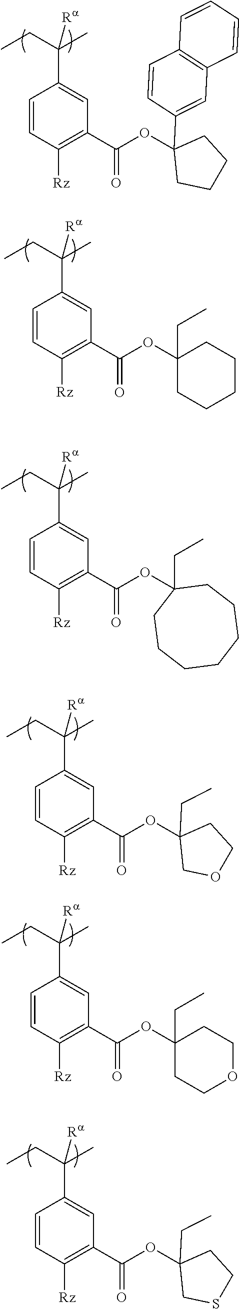

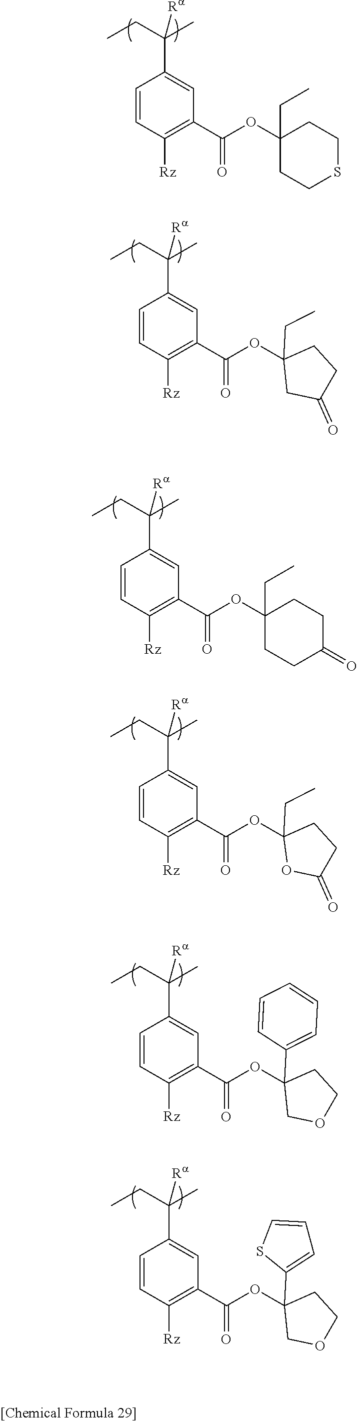

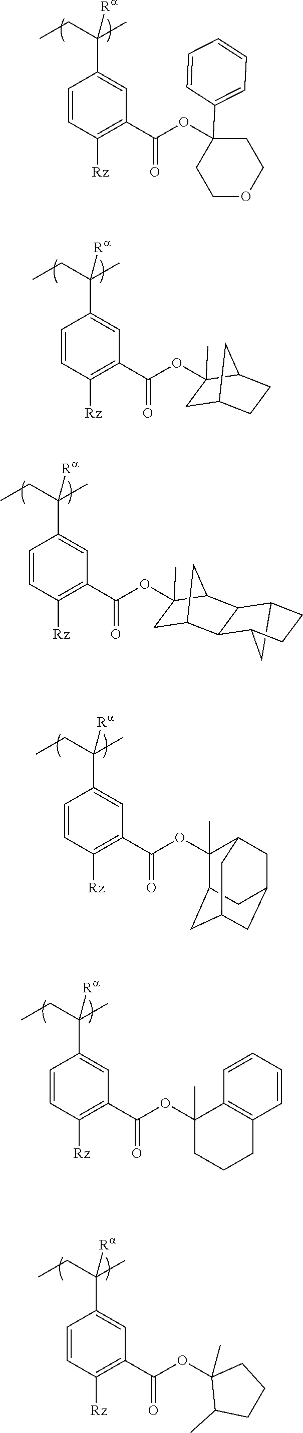

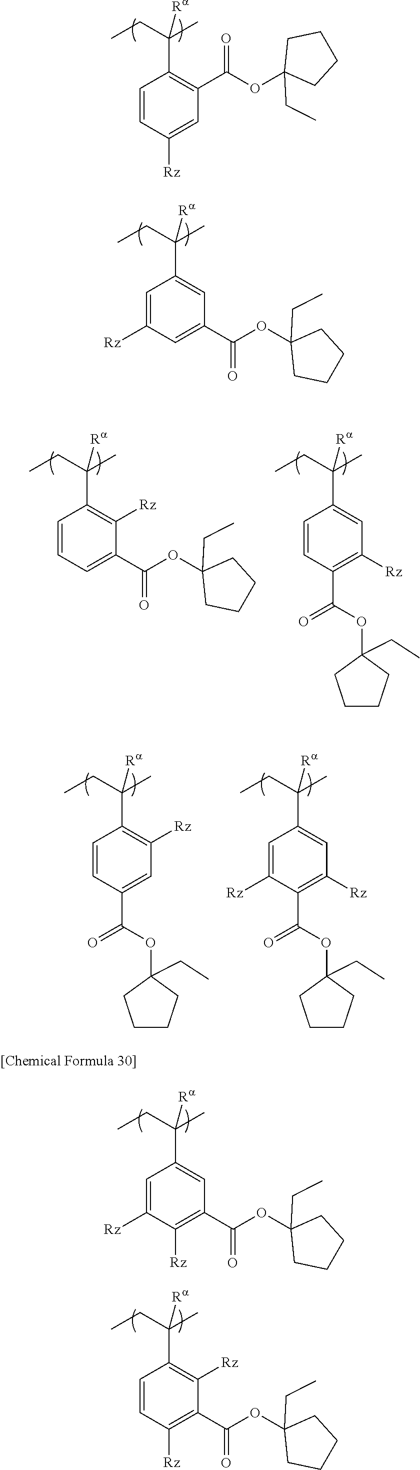

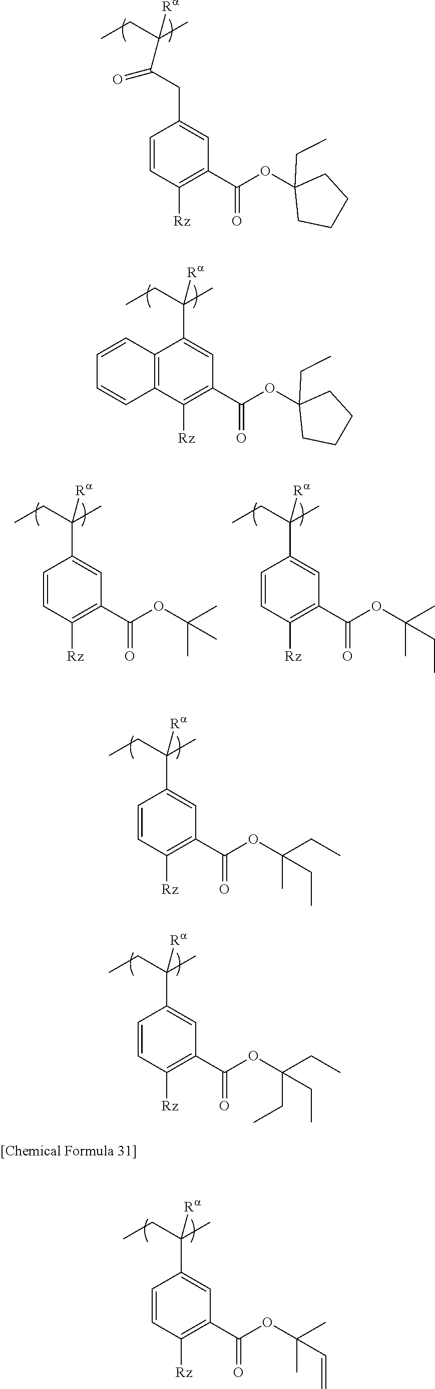

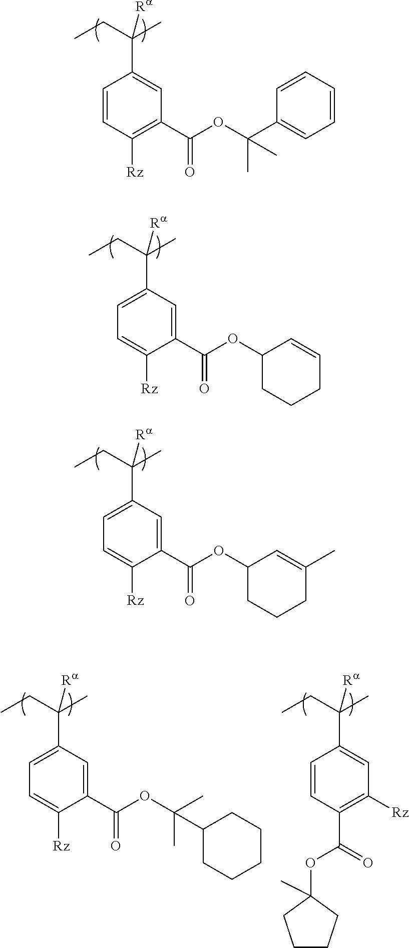

The constitutional unit (a1) included in the component (A1) may be used alone or two or more kinds thereof may be used.

As the constitutional unit (a1), a constitutional unit represented by Formula (a1-1) or Formula (a1-3) is more preferable from the viewpoint that the lithography characteristics (the sensitivity, the shape, and the like) using electron beams or EUV are likely to be enhanced. Among these, from the viewpoint of suitably increasing the reactivity for EB or EUV, the acid dissociable groups (Ra¹, Rax⁰¹) are each preferably an acid dissociable group represented by General Formula (a1-r2-1), (a1-r2-3), (a1-r2-4), or (a1-r-4). Among these, it is particularly preferable to select a cyclic group.

Alternatively, the constitutional unit (a1) may include a constitutional unit represented by General Formula (a1-1-1).

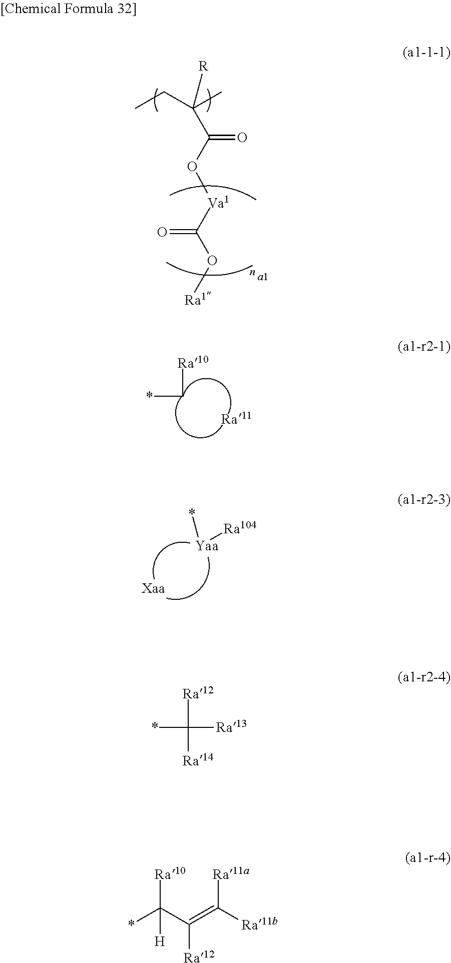

[In the formulae, Ra¹″ represents an acid dissociable group represented by General Formula (a1-r2-1), (a1-r2-3), (a1-r2-4), or (a1-r-4). * represents a bonding site.]

In Formula (a1-1-1), R, Va¹, and nₐ₁ each have the same definition as that for R, Va¹, and nₐ₁ in Formula (a1-1).

The acid dissociable group represented by General Formula (a1-r2-1), (a1-r2-3), (a1-r2-4), or (a1-r-4) is as described above. Among these, it is preferable to select those in which the acid dissociable group is a cyclic group because the reactivity is enhanced for EB or EUV, which is preferable.

The proportion of the constitutional unit (a1) in the component (A1) is preferably in a range of 5% to 80% by mole, more preferably in a range of 10% to 75% by mole, still more preferably in a range of 30% to 70% by mole, and particularly preferably in a range of 40% to 70% by mole with respect to the total amount (100% by mole) of all constitutional units constituting the component (A1).

By setting the proportion of the constitutional unit (a1) to be greater than or equal to the lower limits of the above-described preferable ranges, lithography characteristics such as the sensitivity, the resolution, and improved roughness are improved. Further, in a case where the proportion of the constitutional unit (a1) is set to be less than or equal to the upper limits of the above-described preferable ranges, the constitutional unit (a1) and other constitutional units can be balanced, and the lithography characteristics are improved.

### <<Other Constitutional Units>

The component (A1) may have other constitutional units as necessary in addition to the constitutional unit (a1) described above.

Examples of the other constitutional units include a constitutional unit represented by General Formula (a10-1), a constitutional unit (a2) containing a lactone-containing cyclic group, a —SO₂-containing cyclic group, or a carbonate-containing cyclic group, and a constitutional unit derived from a compound represented by General Formula (a8-1).

Constitutional Unit (a10):

The constitutional unit (a10) is a constitutional unit represented by General Formula (a10-1).

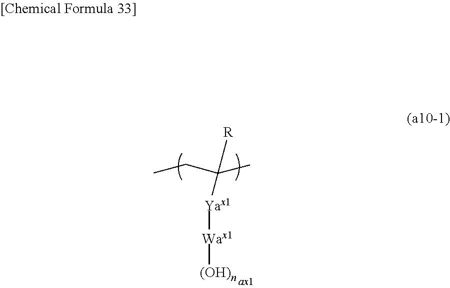

[In the formula, R represents a hydrogen atom, an alkyl group having 1 to 5 carbon atoms, or a halogenated alkyl group having 1 to 5 carbon atoms. Ya^{x1} represents a single bond or a divalent linking group. Wa^{x1} represents an aromatic hydrocarbon group which may have a substituent. nₐₓ₁ represents an integer of 1 or greater.]

In Formula (a10-1), R has the same definition as that for R in General Formula (a1-1). R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of industrial availability.

In Formula (a10-1), Ya^{x1} represents a single bond or a divalent linking group.

In the chemical formula, the divalent linking group as Ya^{x1} is not particularly limited, and suitable examples thereof include a divalent hydrocarbon group which may have a substituent and a divalent linking group having a heteroatom.

Divalent Hydrocarbon Group which May have Substituent:

The divalent hydrocarbon group which may have a substituent may be an aliphatic hydrocarbon group or an aromatic hydrocarbon group.

### Aliphatic Hydrocarbon Group

The aliphatic hydrocarbon group indicates a hydrocarbon group that has no aromaticity. The aliphatic hydrocarbon group may be saturated or unsaturated. In general, it is preferable that the aliphatic hydrocarbon group is saturated.

Examples of the aliphatic hydrocarbon group include a linear or branched aliphatic hydrocarbon group and an aliphatic hydrocarbon group having a ring in the structure thereof.

### Linear or Branched Aliphatic Hydrocarbon Group

The linear aliphatic hydrocarbon group has preferably 1 to 10 carbon atoms, more preferably 1 to 6 carbon atoms, still more preferably 1 to 4 carbon atoms, and most preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃) (CH₂CH₃)—, —C(CH₃) (CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

The linear or branched aliphatic hydrocarbon group may or may not have a substituent. Examples of the substituent include a fluorine atom, a fluorinated alkyl group having 1 to 5 carbon atoms which has been substituted with a fluorine atom, and a carbonyl group.

### Aliphatic Hydrocarbon Group Having Ring in Structure Thereof

Examples of the aliphatic hydrocarbon group having a ring in the structure thereof include a cyclic aliphatic hydrocarbon group which may have a substituent having a heteroatom in the ring structure thereof (a group in which two hydrogen atoms have been removed from an aliphatic hydrocarbon ring), a group in which the cyclic aliphatic hydrocarbon group is bonded to the terminal of a linear or branched aliphatic hydrocarbon group, and a group in which the cyclic aliphatic hydrocarbon group is interposed in the middle of a linear or branched aliphatic hydrocarbon group. As the linear or branched aliphatic hydrocarbon group, the same groups as those described above are exemplary examples.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group obtained by removing two hydrogen atoms from a monocycloalkane. The number of carbon atoms in the monocycloalkane is preferably 3 to 6, and specifically, cyclopentane and cyclohexane are exemplary examples. As the polycyclic alicyclic hydrocarbon group, a group formed by removing two hydrogen atoms from a polycycloalkane is preferable. The number of carbon atoms in the polycycloalkane is preferably 7 to 12, and specifically, adamantane, norbornane, isobornane, tricyclo[5.2.1.0^{2,6}]decane, and tetracyclododecane are exemplary examples.

The cyclic aliphatic hydrocarbon group may or may not have a substituent. Examples of the substituent include an alkyl group, an alkoxy group, a halogen atom, a halogenated alkyl group, a hydroxyl group, and a carbonyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is still more preferable.

As the halogen atom as the substituent, a fluorine atom is preferable.

Examples of the halogenated alkyl group as the substituent include groups in which some or all hydrogen atoms in the above-described alkyl groups are substituted with the above-described halogen atoms.

In the cyclic aliphatic hydrocarbon group, some carbon atoms constituting the ring structure thereof may be substituted with a substituent having a heteroatom. As the substituent having a heteroatom, —O—, —C(═O)—O—, —S—, —S(═O)₂—, or —S(═O)₂—O— is preferable.

### Aromatic Hydrocarbon Group

The aromatic hydrocarbon group is a hydrocarbon group having at least one aromatic ring.

The aromatic ring is not particularly limited as long as the aromatic ring is a cyclic conjugated system having (4n+2) π electrons and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms. Here, the number of carbon atoms in a substituent is not included in the number of carbon atoms.

Specifically, as the aromatic ring, an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms are exemplary examples. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms have been removed from the above-described aromatic hydrocarbon ring or aromatic heterocyclic ring (an arylene group or a heteroarylene group); a group in which two hydrogen atoms have been removed from an aromatic compound having two or more aromatic rings (for example, biphenyl or fluorene); and a group in which one hydrogen atom of a group (an aryl group or a heteroaryl group) obtained by removing one hydrogen atom from the above-described aromatic hydrocarbon ring or aromatic heterocyclic ring has been substituted with an alkylene group (for example, a group obtained by further removing one hydrogen atom from an aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms in the alkylene group bonded to the aryl group or the heteroaryl group is preferably in a range of 1 to 4, more preferably 1 or 2, and particularly preferably 1.

In the aromatic hydrocarbon group, the hydrogen atom in the aromatic hydrocarbon group may be substituted with a substituent. For example, the hydrogen atom bonded to the aromatic ring in the aromatic hydrocarbon group may be substituted with a substituent. Examples of substituents include an alkyl group, an alkoxy group, a halogen atom, a halogenated alkyl group, and a hydroxyl group.

As the alkyl group as the substituent, an alkyl group having 1 to 5 carbon atoms is preferable, and a methyl group, an ethyl group, a propyl group, an n-butyl group, or a tert-butyl group is more preferable.

As the alkoxy group, the halogen atom, and the halogenated alkyl group as the substituents, the groups described as the substituents that substitute a hydrogen atom in the cyclic aliphatic hydrocarbon group are exemplary examples.

### Divalent Linking Group Having Heteroatom:

Examples of the divalent linking group having a heteroatom include —O—, —C(═O)—O—, —O—C(═O)—, —C(═O)—, —O—C(═O)—O—, —C(═O)—NH—, —NH—, —NH—C(═NH)—(H may be substituted with a substituent such as an alkyl group or an acyl group), —S—, —S(═O)₂—, —S(═O)₂—O—, and a group represented by General Formula —Y²¹—O—Y²²—, —Y²¹—O—, —Y²¹—C(═O)—O—, —C(═O)—O—Y²¹—, —[Y²¹—C(═O)—O]_{m″}—Y²², —Y²¹—O—C(═O)—Y²², or —Y²¹—S(═O)₂—O—Y²²— [in the formulae, Y²¹ and Y²² each independently represent a divalent hydrocarbon group which may have a substituent, O represents an oxygen atom, and m″ represents an integer of 1 to 3].

In a case where the above-described divalent linking group containing a heteroatom is —C(═O)—NH—, —C(═O)—NH—C(═O)—, —NH—, or —NH—C(═NH)—, H may be substituted with a substituent such as an alkyl group and an acyl group. The substituent (alkyl group, acyl group, and the like) preferably has 1 to 10 carbon atoms, more preferably has 1 to 8 carbon atoms, and particularly preferably has 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²—, or —Y²¹—S(═O)₂—O—Y²², Y²¹ and Y²² each independently represent a divalent hydrocarbon group which may have a substituent. Examples of the divalent hydrocarbon group include the same groups as described above.

As Y²¹, a linear aliphatic hydrocarbon group is preferable, a linear alkylene group is more preferable, a linear alkylene group having 1 to 5 carbon atoms is still more preferable, and a methylene group or an ethylene group is particularly preferable.

As Y²², a linear or branched aliphatic hydrocarbon group is preferable, and a methylene group, an ethylene group, or an alkylmethylene group is more preferable. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ represents an integer of 1 to 3, preferably an integer of 1 or 2, and more preferably 1. That is, a group represented by Formula —Y²¹—C(═O)—O—Y²²— is preferable as the group represented by Formula —[Y²¹—C(═O)—O]_{m″}—Y²²—. Among these, a group represented by Formula —(CH₂)_{a′}—C(═O)—O—(CH₂)_{b′}— is preferable. In the formula, a′ represents an integer of 1 to 10, preferably an integer of 1 to 8, more preferably an integer of 1 to 5, still more preferably 1 or 2, and most preferably 1. b′ represents an integer of 1 to 10, preferably an integer of 1 to 8, more preferably an integer of 1 to 5, still more preferably 1 or 2, and most preferably 1.

Ya^{x1} represents preferably a single bond, an ester bond [—C(═O)—O— or —O—C(═O)—], an ether bond (—O—), a linear or branched alkylene group, or a combination thereof and more preferably a single bond or an ester bond [—C(═O)—O— or —O—C(═O)—].

In Formula (a10-1), Wa^{x1} represents an aromatic hydrocarbon group which may have a substituent.

Examples of the aromatic hydrocarbon group as Wa^{x1} include a group obtained by removing (nₐₓ₁+1) hydrogen atoms from an aromatic ring which may have a substituent. The aromatic ring is not particularly limited as long as the aromatic ring is a cyclic conjugated system having (4n+2) π electrons. The aromatic ring has preferably 5 to 30 carbon atoms, more preferably 5 to 20 carbon atoms, still more preferably 6 to 15 carbon atoms, and particularly preferably 6 to 12 carbon atoms. Specific examples of the aromatic ring include aromatic hydrocarbon rings such as benzene, naphthalene, anthracene, and phenanthrene; and an aromatic heterocyclic ring obtained by substituting some carbon atoms constituting the above-described aromatic hydrocarbon ring with a heteroatom. Examples of the heteroatom in the aromatic heterocyclic rings include an oxygen atom, a sulfur atom, and a nitrogen atom. Specific examples of the aromatic heterocyclic ring include a pyridine ring and a thiophene ring.

Examples of the aromatic hydrocarbon group as Wa^{x1} also include a group obtained by removing (nₐₓ₁+1) hydrogen atoms from an aromatic compound including an aromatic ring (for example, biphenyl or fluorene) which may have two or more substituents.

Among these, Wa^{x1} represents preferably a group in which (nₐₓ₁+1) hydrogen atoms have been removed from benzene, naphthalene, anthracene, or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group as Wa^{x1} may have a substituent or may not have a substituent. Examples of the substituent include an alkyl group, an alkoxy group, a halogen atom, and a halogenated alkyl group. Examples of the alkyl group, the alkoxy group, the halogen atom, and the halogenated alkyl group, as the substituent, include the same groups as those for the above-described substituent of the cyclic aliphatic hydrocarbon group as Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. It is preferable that the aromatic hydrocarbon group as Wa^{x1} has no substituent.

In Formula (a10-1), nₐₓ₁ represents an integer of 1 or greater, preferably an integer of 1 to 10, more preferably an integer of 1 to 5, still more preferably 1, 2, or 3, and particularly preferably 1 or 2.

Specific examples of the constitutional unit (a10) represented by Formula (a10-1) are described below.

In the formulae shown below, R^{a} represents a hydrogen atom, a methyl group, or a trifluoromethyl group.

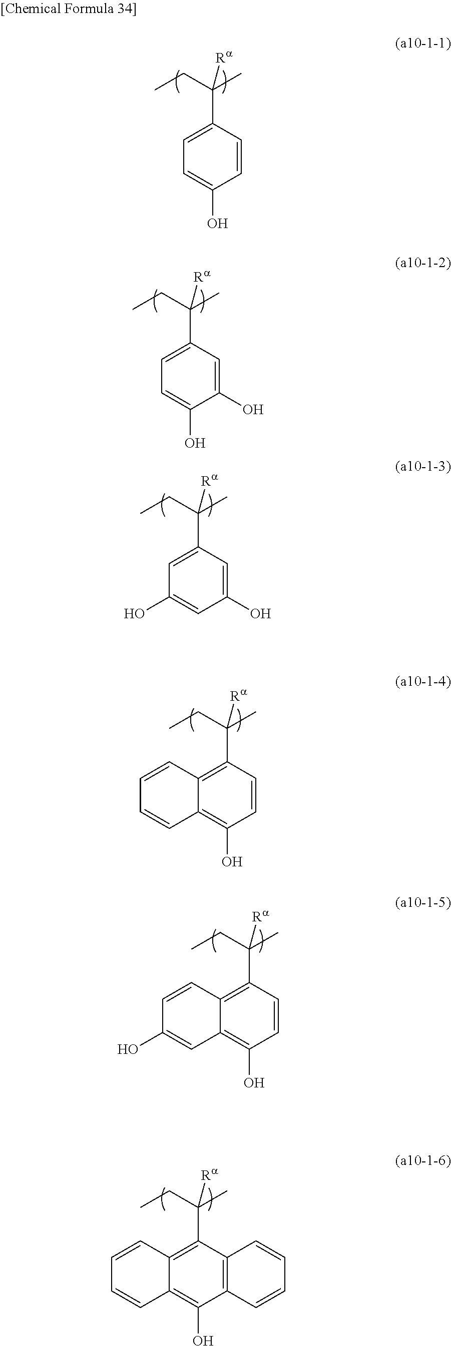

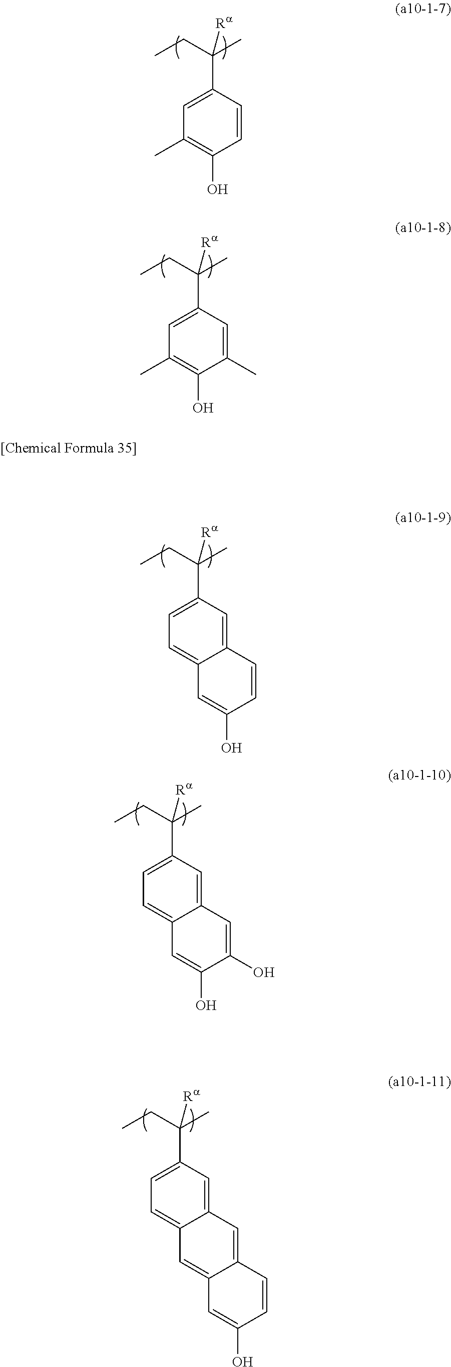

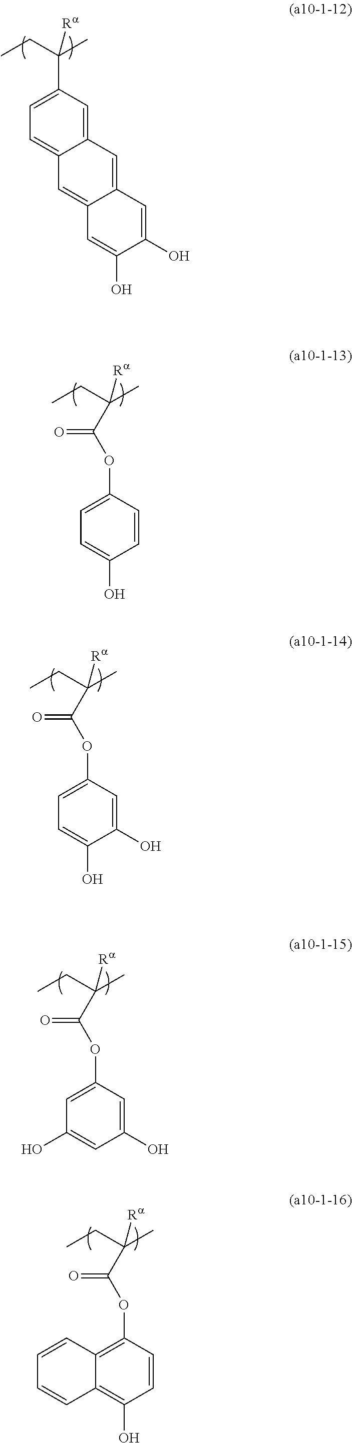

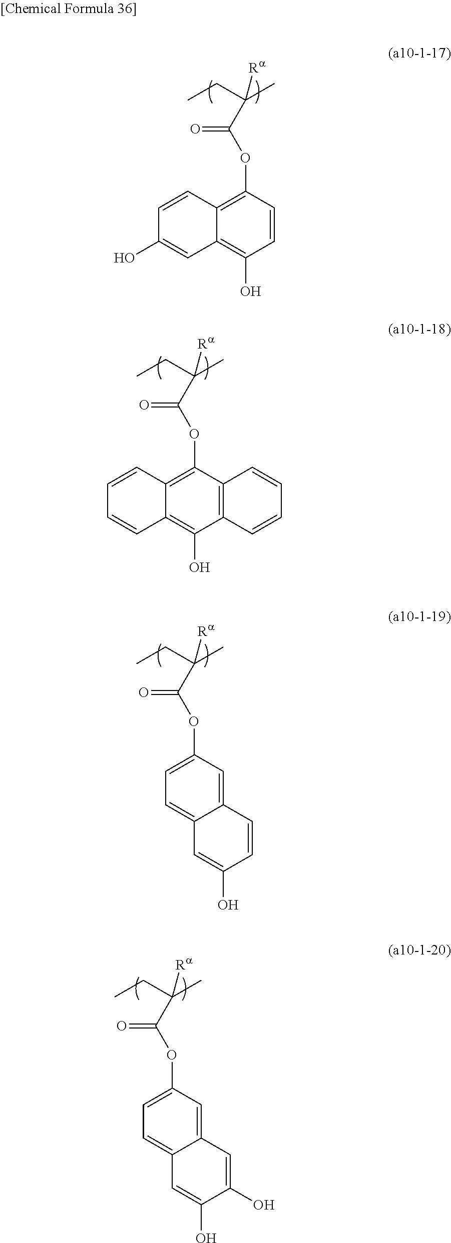

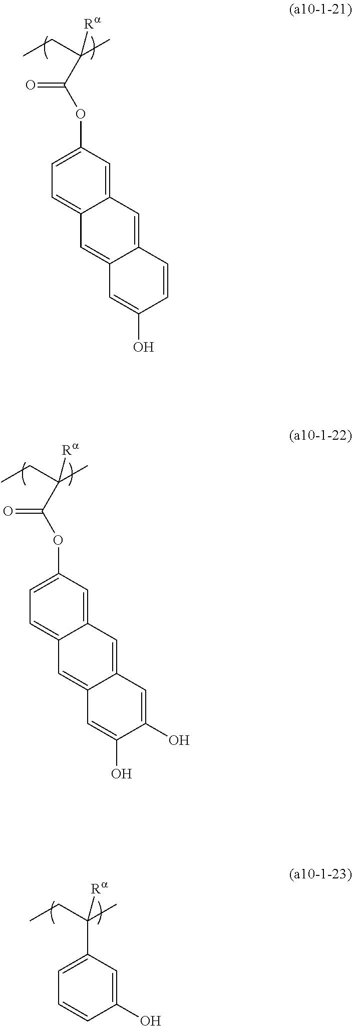

The constitutional unit (a10) included in the component (A1) may be used alone or two or more kinds thereof may be used. The component (A1) may or may not have the constitutional unit (a10), but it is preferable that the component (A1) has the constitutional unit (a10).

In a case where the component (A1) has the constitutional unit (a10), the proportion of the constitutional unit (a10) in the component (A1) is preferably in a range of 20% to 80% by mole, more preferably in a range of 25% to 70% by mole, still more preferably in a range of 30% to 60% by mole, and particularly preferably in a range of 40% to 60% by mole with respect to the total amount (100% by mole) of all constitutional units constituting the component (A1).

In a case where the proportion of the constitutional unit (a10) is set to be greater than or equal to the above-described lower limits, the sensitivity is likely to be enhanced. Meanwhile, in a case where the proportion thereof is set to be less than or equal to the above-described upper limits, the constitutional unit (a10) and other constitutional units are likely to be balanced.

### Constitutional Unit (a2):

The component (A1) may have a constitutional unit (a2) containing a lactone-containing cyclic group (here, constitutional units corresponding to the constitutional unit (a1) are excluded).

In a case where the component (A1) is used to form a resist film, the lactone-containing cyclic group of the constitutional unit (a2) is effective for increasing the adhesiveness of the resist film to the substrate. Further, in a case where the component (A1) contains the constitutional unit (a2), the lithography characteristics and the like are improved due to the effects of appropriately adjusting the acid diffusion length, increasing the adhesiveness of the resist film to the substrate, and appropriately adjusting the solubility during development.

The term “lactone-containing cyclic group” indicates a cyclic group that has a ring (lactone ring) containing —O—C(═O)— in the ring skeleton. In a case where the lactone ring is counted as the first ring and the group contains only the lactone ring, the group is referred to as a monocyclic group. Further, in a case where the group has other ring structures, the group is referred to as a polycyclic group regardless of the structures. The lactone-containing cyclic group may be a monocyclic group or a polycyclic group.

The lactone-containing cyclic group in the constitutional unit (a2) is not particularly limited, and an optional constitutional unit can be used. Specific examples thereof include groups each represented by General Formulae (a2-r-1) to (a2-r-7).

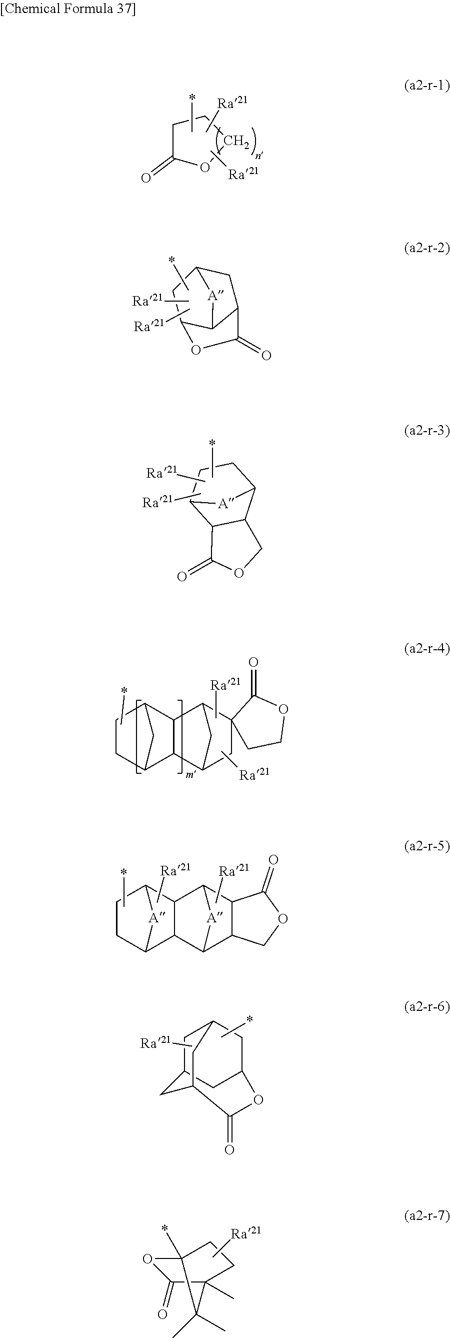

[In the formulae, Ra′²¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group, R″ represents a hydrogen atom, an alkyl group, or a lactone-containing cyclic group, A″ represents an alkylene group having 1 to 5 carbon atoms which may have an oxygen atom (—O—) or a sulfur atom (—S—), an oxygen atom, or a sulfur atom, n′ represents an integer of 0 to 2, and m′ represents 0 or 1. * represents a bonding site (the same applies hereinafter).]

In General Formulae (a2-r-1) to (a2-r-7), it is preferable that the alkyl group as Ra′²¹ is an alkyl group having 1 to 6 carbon atoms. Further, it is preferable that the alkyl group is linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or ethyl group is preferable, and a methyl group is particularly preferable.

It is preferable that the alkoxy group as Ra′²¹ is an alkoxy group having 1 to 6 carbon atoms. Further, it is preferable that the alkoxy group is linear or branched. Specific examples of the alkoxy groups include a group formed by linking the above-described alkyl group described as the alkyl group represented by Ra′²¹ to an oxygen atom (—O—).

As the halogen atom as Ra′²¹, a fluorine atom is preferable.

Examples of the halogenated alkyl group as Ra′²¹ include groups in which some or all hydrogen atoms in the alkyl group as Ra′²¹ have been substituted with the halogen atoms. As the halogenated alkyl group, a fluorinated alkyl group is preferable, and a perfluoroalkyl group is particularly preferable.

In —COOR″ and —OC(═O)R″ as Ra′²¹, each R″ represents a hydrogen atom, an alkyl group, or a lactone-containing cyclic group.

The alkyl group as R″ may be linear, branched, or cyclic and has preferably 1 to 15 carbon atoms.

In a case where R″ represents a linear or branched alkyl group, an alkyl group having 1 to 10 carbon atoms is preferable, an alkyl group having 1 to 5 carbon atoms is more preferable, and a methyl group or an ethyl group is particularly preferable.

In a case where R″ represents a cyclic alkyl group, the number of carbon atoms thereof is preferably in a range of 3 to 15, more preferably in a range of 4 to 12, and most preferably in a range of 5 to 10. Specific examples thereof include groups in which one or more hydrogen atoms have been removed from a monocycloalkane, which may or may not be substituted with a fluorine atom or a fluorinated alkyl group; and groups in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclo[5.2.1.0^{2,6}]decane, or tetracyclododecane.

Examples of the lactone-containing cyclic group as R″ include the same groups as those for the groups each represented by General Formulae (a2-r-1) to (a2-r-7).

As the hydroxyalkyl group as Ra′²¹, a hydroxyalkyl group having 1 to 6 carbon atoms is preferable, and specific examples thereof include a group in which at least one hydrogen atom in the alkyl group as Ra′²¹ has been substituted with a hydroxyl group.

Among the examples, it is preferable that each Ra′²¹ independently represent a hydrogen atom or a cyano group.

In General Formulae (a2-r-2), (a2-r-3) and (a2-r-5), as the alkylene group having 1 to 5 carbon atoms as A″, a linear or branched alkylene group is preferable, and examples thereof include a methylene group, an ethylene group, an n-propylene group, and an isopropylene group. Specific examples of the alkylene groups that contain an oxygen atom or a sulfur atom include a group obtained by interposing —O— or —S— in the terminal of the alkylene group or between the carbon atoms of the alkylene group, and examples thereof include —O—CH₂—, —CH₂—O—CH₂—, —S—CH₂—, and —CH₂—S—CH₂—. A″ represents preferably an alkylene group having 1 to 5 carbon atoms or —O—, more preferably an alkylene group having 1 to 5 carbon atoms, and most preferably a methylene group.

Specific examples of the groups each represented by General Formulae (a2-r-1) to (a2-r-7) are shown below.

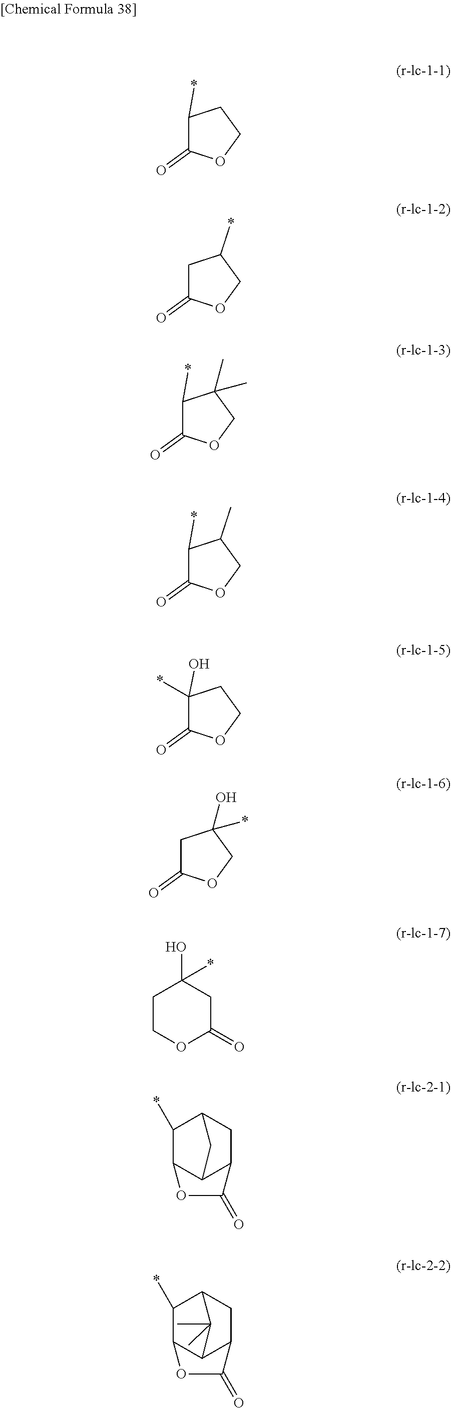

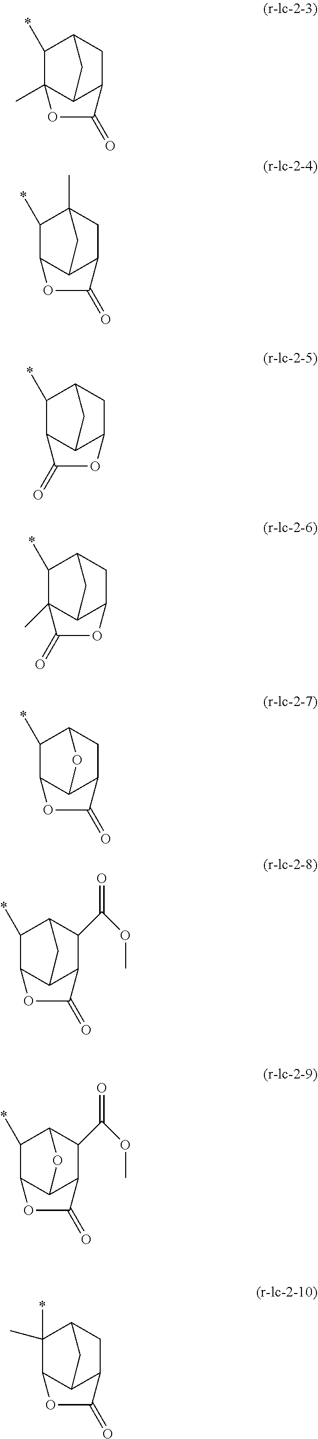

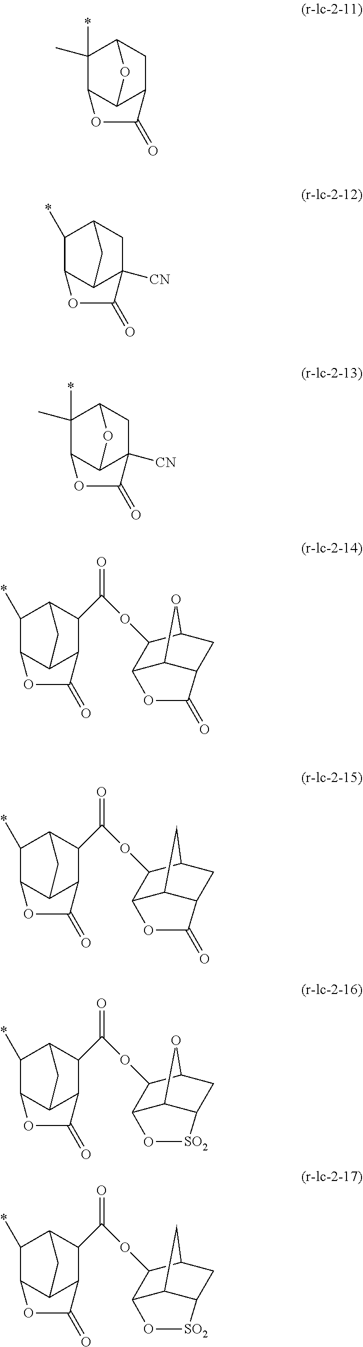

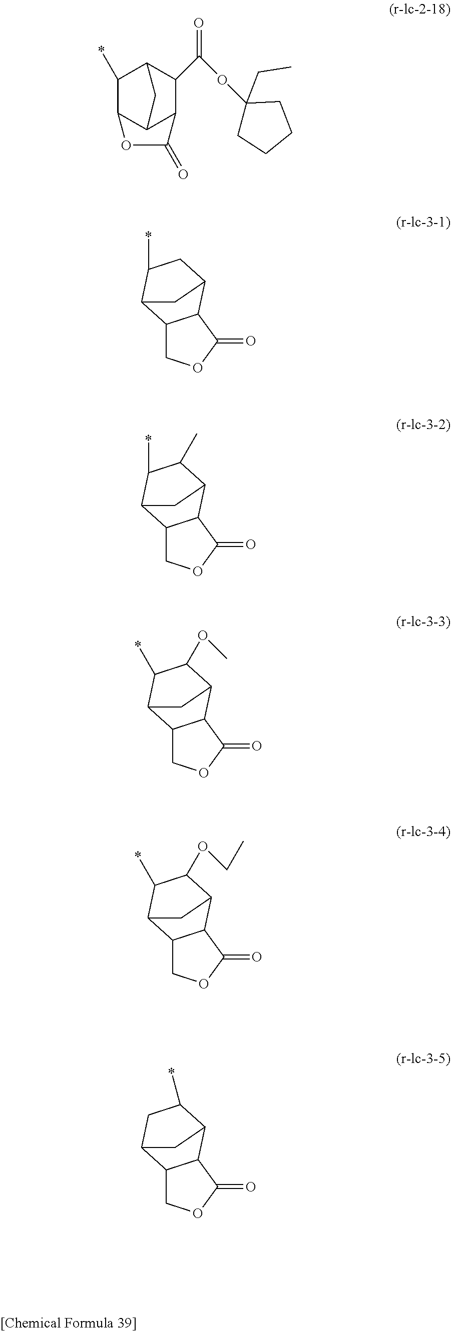

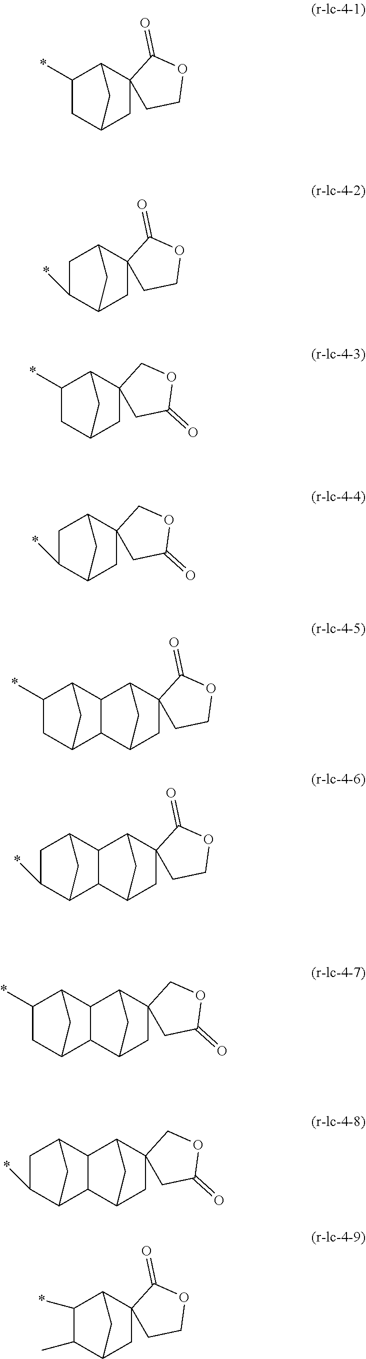

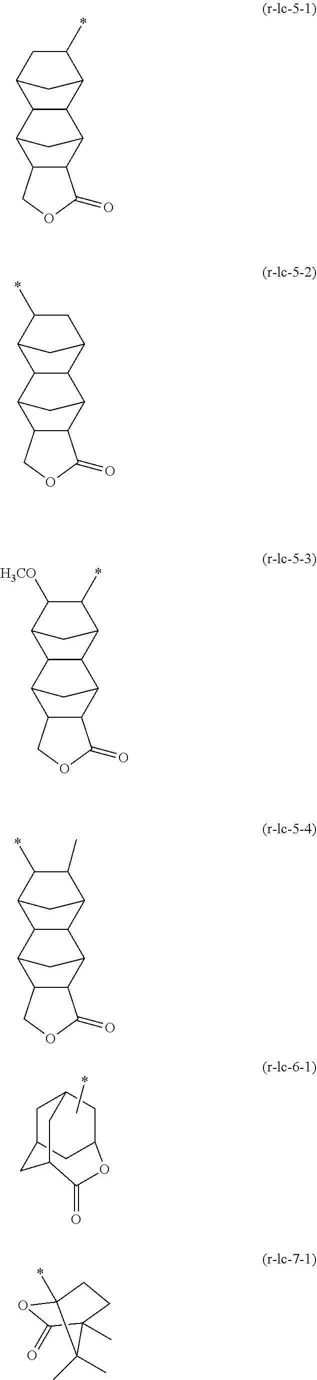

As the constitutional unit (a2), a constitutional unit derived from acrylic acid ester in which the hydrogen atom bonded to the carbon atom at the α-position may be substituted with a substituent is preferable.

It is preferable that such a constitutional unit (a2) is a constitutional unit represented by General Formula (a2-1).

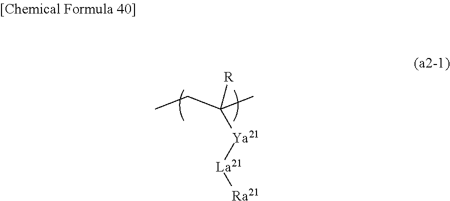

[In the formula, R represents a hydrogen atom, an alkyl group having 1 to 5 carbon atoms, or a halogenated alkyl group having 1 to 5 carbon atoms. Ya²¹ represents a single bond or a divalent linking group. La²¹ represents —O—, —COO—, —CON(R′)—, —OCO—, —CONHCO—, or —CONHCS—, and R′ represents a hydrogen atom or a methyl group. In a case where La²¹ represents —O—, Ya²¹ does not represent —CO—. Ra²¹ represents a lactone-containing cyclic group.]

In Formula (a2-1), R has the same definition as described above. R represents preferably a hydrogen atom, an alkyl group having 1 to 5 carbon atoms, or a fluorinated alkyl group having 1 to 5 carbon atoms and particularly preferably a hydrogen atom or a methyl group from the viewpoint of industrial availability.

In Formula (a2-1), the divalent linking group as Ya²¹ is not particularly limited, and suitable examples thereof include a divalent hydrocarbon group which may have a substituent and a divalent linking group having a heteroatom. Examples of the divalent linking group as Ya²¹ include the same groups as those for the divalent linking group as Ya^{x1} in General Formula (a10-1) described above.

It is preferable that Ya²¹ represents a single bond, an ester bond [—C(═O)—O—], an ether bond (—O—), a linear or branched alkylene group, or a combination thereof.

In Formula (a2-1), it is preferable that Ya²¹ represents a single bond and La²¹ represents —COO— or —OCO—.

In Formula (a2-1), Ra²¹ represents a lactone-containing cyclic group.

Suitable examples of the lactone-containing cyclic group as Ra²¹ include groups each represented by General Formulae (a2-r-1) to (a2-r-7).

The constitutional unit (a2) included in the component (A1) may be used alone or two or more kinds thereof may be used. The component (A1) may or may not have the constitutional unit (a2).

In a case where the component (A1) has the constitutional unit (a2), the proportion of the constitutional unit (a2) is preferably in a range of 1% to 20% by mole, more preferably in a range of 1% to 15% by mole, and still more preferably in a range of 1% to 10% by mole with respect to the total amount (100% by mole) of all constitutional units constituting the component (A1).

In a case where the proportion of the constitutional unit (a2) is set to be greater than or equal to the lower limits of the above-described preferable ranges, the effect to be obtained by allowing the component (A1) to have the constitutional unit (a2) is sufficiently obtained by the above-described effects. Further, in a case where the proportion thereof is set to be less than or equal to the upper limits of the above-described preferable ranges, the constitutional unit (a2) and other constitutional units can be balanced, and the lithography characteristics are improved.

### Constitutional Unit (a8):

The constitutional unit (a8) is a constitutional unit derived from a compound represented by General Formula (a8-1).

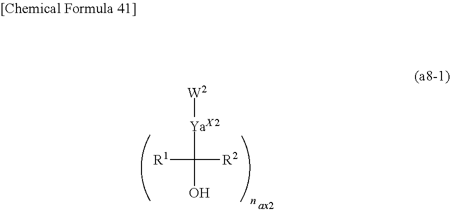

[In the formula, W² represents a polymerizable group-containing group. Ya^{x2} represents a single bond or an (nₐₓ₂+1)-valent linking group. Ya^{x2} and W² may form a condensed ring. R¹ represents a fluorinated alkyl group having 1 to 12 carbon atoms. R² represents an organic group having 1 to 12 carbon atoms which may have a fluorine atom, or a hydrogen atom. R² and Ya^{x2} may be bonded to each other to form a ring structure. nₐₓ₂ represents an integer of 1 to 3.]

The term “polymerizable group” in the polymerizable group-containing group as W² is a group that enables a compound having the polymerizable group to be polymerized by radical polymerization or the like, and refers to a group containing multiple bonds between carbon atoms, such as an ethylenic double bond.

The polymerizable group-containing group may be a group formed of only a polymerizable group or a group formed of a polymerizable group and a group other than the polymerizable group. Examples of the group other than the polymerizable group include a divalent hydrocarbon group which may have a substituent and a divalent linking group containing a heteroatom.

Suitable examples of the polymerizable group-containing group include a group represented by a chemical formula: C(R^{X11})(R^{X12})═C(R^{X13})-Ya^{x0}-.

In the chemical formula, R^{X11}, R^{X12}, and R^{X13} each represent a hydrogen atom, an alkyl group having 1 to 5 carbon atoms, or a halogenated alkyl group having 1 to 5 carbon atoms, and Ya^{x0} represents a single bond or a divalent linking group.

Examples of the condensed ring formed by Ya^{x2} and W² include a condensed ring formed by a polymerizable group of the W² moiety and by Ya^{x2} and a condensed ring formed by a group other than the polymerizable group of the W² moiety and by Ya^{x2}.

The condensed ring formed by Ya^{x2} and W² may have a substituent.

Specific examples of the constitutional unit (a8) are shown below.

In the following formulae, R^{a} represents a hydrogen atom, a methyl group, or a trifluoromethyl group.

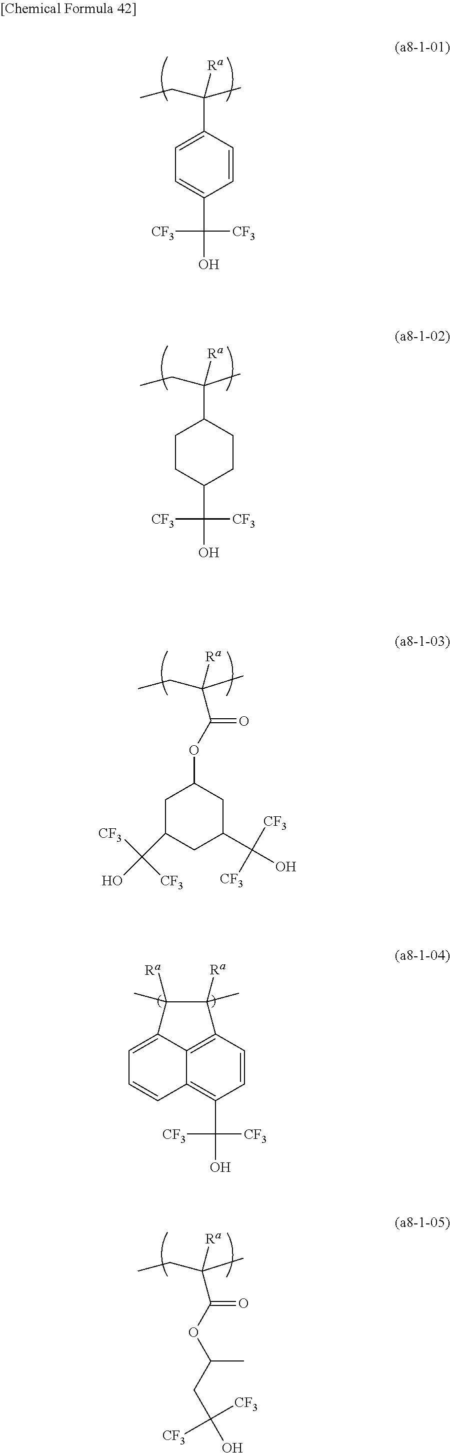

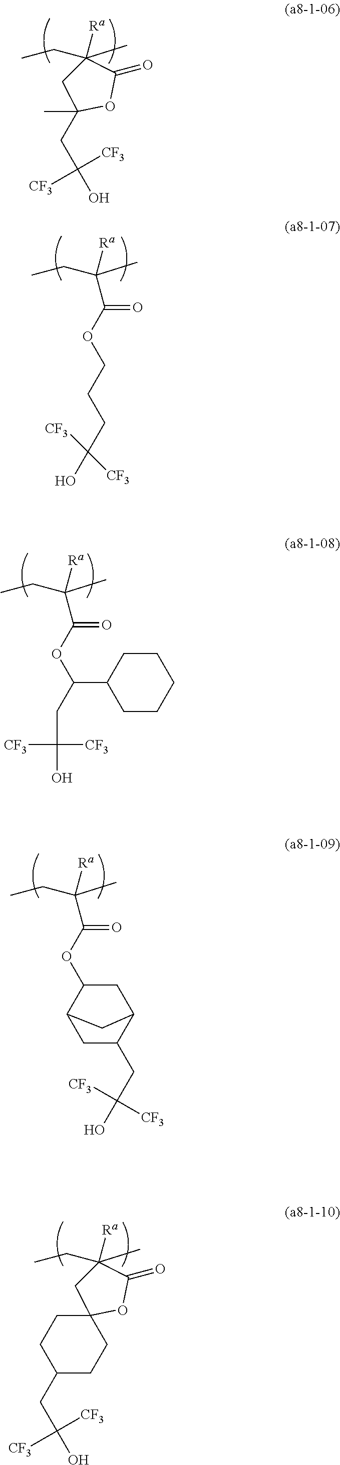

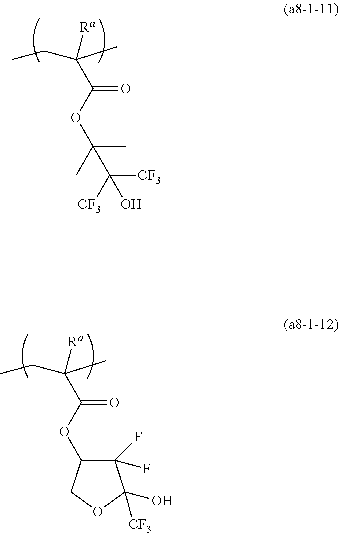

Among the examples, the constitutional unit (a8) is preferably at least one selected from the group consisting of constitutional units each represented by Chemical Formulae (a8-1-01) to (a8-1-04), (a8-1-06), (a8-1-08), (a8-1-09), and (a8-1-10) and more preferably at least one selected from the group consisting of constitutional units each represented by Chemical Formulae (a8-1-01) to (a8-1-04) and (a8-1-09).

The constitutional unit (a8) contained in the component (A1) may be used alone or two or more kinds thereof may be used. The component (A1) may or may not have the constitutional unit (a8).

The proportion of the constitutional unit (a8) in the component (A1) is preferably in a range of 0% to 50% by mole and more preferably in a range of 0% to 30% by mole with respect to the total amount (100% by mole) of all constitutional units constituting the component (A1).

The component (A1) contained in the resist composition may be used alone or a combination of two or more kinds thereof may be used.

Examples of the component (A1) include a polymer compound having a repeating structure of the constitutional unit (a1) and the constitutional unit (a10).

Such a component (A1) can be produced by dissolving a monomer, from which each constitutional unit is derived, in a polymerization solvent and adding a radical polymerization initiator such as azobisisobutyronitrile (AIBN) or dimethyl azobisisobutyrate (for example, V-601) to the solution so that the polymerization is carried out.

Alternatively, such a component (A1) can be produced by dissolving, in a polymerization solvent, a monomer from which the constitutional unit (a1) is derived and a monomer from which an optional constitutional unit (for example, the constitutional unit (a10) or the like) is derived, adding a radical polymerization initiator as described above thereto to carry out polymerization, and carrying out a deprotection reaction.

Further, a —C(CF₃)₂—OH group may be introduced into the terminal of the component (A1) during the polymerization using a chain transfer agent such as HS—CH₂—CH₂—CH₂—C(CF₃)₂—OH in combination. As described above, a copolymer into which a hydroxyalkyl group, formed by substitution of some hydrogen atoms in the alkyl group with fluorine atoms, has been introduced is effective for reducing development defects and reducing line edge roughness (LER: uneven irregularities of a line side wall).

The weight-average molecular weight (Mw) (in terms of polystyrene according to gel permeation chromatography (GPC)) of the component (A1) is not particularly limited, but is preferably in a range of 1000 to 50000, more preferably in a range of 5000 to 40000, and still more preferably in a range of 5000 to 30000.

In a case where the Mw of the component (A1) is less than or equal to the upper limits of the above-described preferable ranges, the resist composition exhibits a satisfactory solubility in a resist solvent for a resist enough to be used as a resist. On the contrary, in a case where the Mw of the component (A1) is greater than or equal to the lower limits of the above-described preferable ranges, the dry etching resistance and the cross-sectional shape of the resist pattern are excellent.

Further, the dispersity (Mw/Mn) of the component (A1) is not particularly limited, but is preferably in a range of 1.0 to 4.0, more preferably in a range of 1.0 to 3.0, and particularly preferably in a range of 1.0 to 2.0. Further, Mn represents the number average molecular weight.

### In Regard to Component (A2)

In the resist composition of the present embodiment, a base material component (hereinafter, also referred to as “component (A2)”) which does not correspond to the component (A1) and whose solubility in a developing solution is changed by the action of an acid may be used in combination as the component (A).

The component (A2) is not particularly limited and may be optionally selected from a plurality of components of the related art which have been known as base material components for a chemically amplified resist composition and used.

As the component (A2), a polymer compound or a low-molecular-weight compound may be used alone or a combination of two or more kinds thereof may be used.

The proportion of the component (A1) in the component (A) is preferably 25% by mass or greater, more preferably 50% by mass or greater, and still more preferably 75% by mass or greater, and may be 100% by mass with respect to the total mass of the component (A). In a case where the proportion thereof is 25% by mass or greater, a resist pattern having excellent various lithography characteristics such as high sensitivity, high resolution, and improved roughness is likely to be formed.

In the resist composition of the present embodiment, the content of the component (A) may be adjusted according to the thickness of the resist film intended to be formed.

### <Acid Generator Component (B)>

The resist composition according to the present embodiment further contains, in addition to the component (A), an acid generator component (component (B)) that generates an acid upon light exposure. The component (B) includes a compound (B0) (hereinafter, also referred to as “component (B0)”) represented by General Formula (b0).

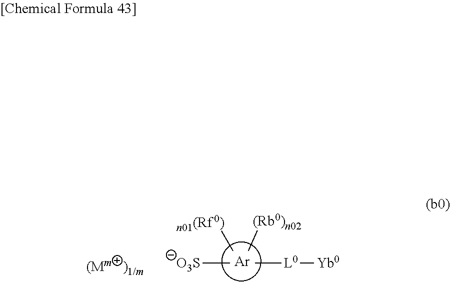

[In the formula, Ar represents an aromatic ring. Rf⁰ represents a fluorinated alkyl group having 1 to 5 carbon atoms or a fluorine atom. L⁰ represents a divalent linking group having —C(═O)—O—, —O—C(═O)—, or —O—S(═O)₂—. Yb⁰ represents a cyclic group. Rb⁰ represents an organic group. n01 represents an integer of 1 or greater as long as a valence is allowed. n02 represents an integer of 0 or greater as long as a valence is allowed. Here, in a case where L⁰-Yb⁰ represents —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of an aromatic ring is substituted with an alkyl group or an alkoxy group. In a case where n01 represents 2 or greater, a plurality of Rf⁰'s may be the same as or different from each other. In a case where n02 represents 2 or greater, a plurality of Rb⁰'s may be the same as or different from each other. m represents an integer of 1 or greater, and M^{m+} represents an m-valent cation.]

### {Anion Moiety}

In Formula (b0), the aromatic ring as Ar is not particularly limited as long as the aromatic ring is a cyclic conjugated system having 4n+2 π electrons, and may be monocyclic or polycyclic. The aromatic ring has preferably 5 to 30 carbon atoms, more preferably 5 to 20 carbon atoms, still more preferably 6 to 15 carbon atoms, and particularly preferably 6 to 12 carbon atoms.

Specific examples of the aromatic ring include an aromatic hydrocarbon ring such as benzene, naphthalene, anthracene, and phenanthrene; and an aromatic heterocyclic ring in which some carbon atoms constituting the aromatic hydrocarbon ring have been substituted with heteroatoms. Examples of the heteroatom in the aromatic heterocyclic rings include an oxygen atom, a sulfur atom, and a nitrogen atom. Specific examples of the aromatic heterocyclic ring include a pyridine ring, a thiophene ring, and a furan ring, but the present invention is not limited thereto.

The aromatic ring as Ar is preferably an aromatic hydrocarbon ring, more preferably a benzene ring or a naphthalene ring, and still more preferably a benzene ring.

In Formula (b0), the fluorinated alkyl group having 1 to 5 carbon atoms as Rf⁰ is a group in which some or all hydrogen atoms of an alkyl group having 1 to 5 carbon atoms are substituted with fluorine atoms. The fluorinated alkyl group may be linear or branched, but is preferably linear.

The linear fluorinated alkyl group as Rf⁰ has preferably 1 to 3 carbon atoms, more preferably 1 or 2 carbon atoms, and still more preferably 1 carbon atom. The branched fluorinated alkyl group as Rf⁰ has preferably 3 or 4 carbon atoms and more preferably 3 carbon atoms.

From the viewpoint of acid strength, the fluorinated alkyl group having 1 to 5 carbon atoms as Rf⁰ is preferably a perfluoroalkyl group and more preferably a trifluoromethyl group.

From the viewpoint of acid strength, Rf⁰ represents preferably a fluorine atom or a trifluoromethyl group and more preferably a fluorine atom.

In Formula (b0), the organic group as Rb⁰ is an organic group other than a fluorinated alkyl group. Examples of the organic group as Rb⁰ include a hydrocarbon group which may have a substituent. The hydrocarbon group may be an aliphatic hydrocarbon group or an aromatic hydrocarbon group, but is preferably an aliphatic hydrocarbon group. The hydrocarbon group may or may not have a substituent, but it is preferable that the hydrocarbon group does not have a substituent.

The aliphatic hydrocarbon group as Rb⁰ has preferably 1 to 10 carbon atoms, more preferably 1 to 6 carbon atoms, still more preferably 1 to 4 carbon atoms, and particularly preferably 1 to 3 carbon atoms. The aliphatic hydrocarbon group as Rb⁰ may be saturated or unsaturated, but is preferably saturated. As the aliphatic hydrocarbon group as Rb⁰, a linear or branched alkyl group is preferable.

The linear alkyl group as Rb⁰ has preferably 1 to 3 carbon atoms, more preferably 1 or 2 carbon atoms, and still more preferably 1 carbon atom. The branched alkyl group as Rb⁰ has preferably 3 or 4 carbon atoms and more preferably 3 carbon atoms.

The organic group as Rb⁰ is preferably a linear or branched alkyl group having 1 to 5 carbon atoms, more preferably a linear or branched alkyl group having 1 to 3 carbon atoms, and still more preferably an ethyl group or a methyl group.

In Formula (b0), n01 represents an integer of 1 or greater as long as the valence is allowed. n01 represents preferably 1 to 4, more preferably 2 to 4, still more preferably 3 or 4, and particularly preferably 4.

In Formula (b0), n02 represents an integer of 0 or greater as long as the valence is allowed. n02 represents preferably 0 to 3, more preferably 0 to 2, still more preferably 0 or 1, and particularly preferably 0.

In Formula (b0), examples of the divalent linking group as L⁰ include —C(═O)—O—, —O—C(═O)—, —O—S(═O)₂—, and a combination of any one or more of these groups and an alkylene group. In a case where the divalent linking group as L⁰ is —C(═O)—O—, —O—C(═O)—, or —O—S(═O)₂—, L⁰-Yb⁰ is —C(═O)—O—Yb⁰, —O—C(═O)—Yb⁰, or —O—S(═O)₂—Yb⁰. In a case where L⁰ represents a combination of any one or more of —C(═O)—O—, —O—C(═O)—, and —O—S(═O)₂— and an alkylene group, the alkylene group may be linear or branched.

Examples of the divalent linking group as L⁰ include a linking group represented by General Formula (L0-1).

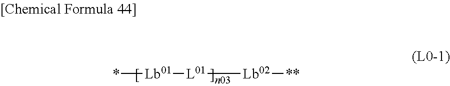

[In the formula, Lb⁰¹ and Lb⁰² each independently represent a single bond or an alkylene group having 1 to 5 carbon atoms, L⁰¹ represents —C(═O)—O—, —O—C(═O)—, or —O—S(═O)₂—, and n03 represents an integer of 1 to 3. Here, in a case where n03 represents 2 or 3, Lb⁰¹ does not represent a single bond. In a case where n03 represents 2 or 3, a plurality of Lb⁰'s may be the same as or different from each other. In a case where n03 represents 2 or greater, a plurality of L⁰¹'s may be the same as or different from each other. * represents a bonding site that is bonded to Ar in Formula (b0), and ** represents a bonding site that is bonded to Yb⁰¹ in Formula (b0).]

In Formula (L0-1), the alkylene group having 1 to 5 carbon atoms as Lb⁰¹ and Lb⁰² may be linear or branched, but is preferably linear. The linear alkylene group as Lb⁰¹ and Lb⁰² has preferably 1 to 3 carbon atoms and more preferably 1 or 2 carbon atoms. The branched alkylene group as Lb⁰¹ and Lb⁰² has preferably 2 to 4 carbon atoms and more preferably 2 or 3 carbon atoms.

In Formula (L0-1), n03 represents preferably 1 or 2 and more preferably 1.

Examples of the divalent linking group as L⁰ include linking groups represented by General Formulae (L01-1) to (L03-1).

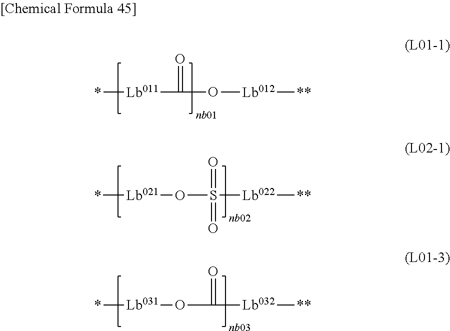

[In the formulae, Lb⁰¹¹ to Lb⁰³¹ each independently represent a single bond or an alkylene group having 1 to 5 carbon atoms. Lb⁰¹² to Lb⁰³² each independently represent a single bond or a divalent linking group. nb01 to nb03 each independently represent an integer of 1 to 3. Here, in a case where nb01 to nb03 represent 2 or 3, Lb⁰¹¹ to Lb⁰³¹ do not represent a single bond. In a case where nb01 represents 2 or 3, a plurality of Lb⁰¹¹'s may be the same as or different from each other. In a case where nb02 represents 2 or 3, a plurality of Lb⁰²¹'s may be the same as or different from each other. In a case where nb03 represents 2 or 3, a plurality of Lb⁰³¹'s may be the same as or different from each other. * represents a bonding site that is bonded to Ar in General Formula (b0), and ** represents a bonding site that is bonded to Yb⁰ in General Formula (b0).]

In Formulae (L01-1) to (L01-3), examples of the alkylene group having 1 to 5 carbon atoms as Lb⁰¹¹ to Lb⁰³¹ include the same groups as those for Lb⁰¹ in Formula (L0-1).

In Formulae (L01-1) to (L01-3), examples of the divalent linking group as Lb⁰¹² to Lb⁰³² include an alkylene group having 1 to 5 carbon atoms, which may have —C(═O)—O—, —O—C(═O)—, or —O—S(═O)₂—. Examples of the alkylene group having 1 to 5 carbon atoms include the same groups as those for Lb⁰¹ in Formula (L0-1).

In Formulae (L01-1) to (L01-3), nb01 to nb03 represent preferably 1 or 2 and more preferably 1.

Examples of L⁰ in Formula (b0) include a group represented by any of General Formulae (L0-1-1) to (L0-1-5).

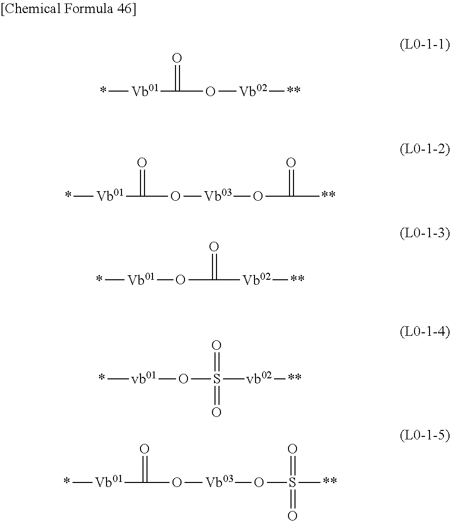

[In the formulae, Vb⁰¹ and Vb⁰² each independently represent a single bond or an alkylene group having 1 to 5 carbon atoms. Vb⁰³ represents an alkylene group having 1 to 5 carbon atoms. * represents a bonding site that is bonded to Ar in Formula (b0), and ** represents a bonding site that is bonded to Yb⁰¹ in Formula (b0).]

In Formulae (L0-1-1) to (L0-1-5), the alkylene group having 1 to 5 carbon atoms as Vb⁰¹ and Vb⁰² may be linear or branched, but is preferably linear. The linear alkylene group as Vb⁰¹ and Vb⁰² has preferably 1 to 3 carbon atoms and more preferably 1 or 2 carbon atoms. The branched alkylene group as Vb⁰¹ and Vb⁰² has preferably 2 to 4 carbon atoms and more preferably 2 or 3 carbon atoms.

### <<In Regard to Yb^{0>>}

Case where L⁰-Yb⁰ does not represent —O—C(═O)—Yb⁰:

In a case where L⁰-Yb⁰ does not represent —O—C(═O)—Yb⁰, Yb⁰ represents a cyclic group.

The cyclic group as Yb⁰ is preferably a cyclic hydrocarbon group. The above-described cyclic hydrocarbon group may be an aliphatic hydrocarbon group or an aromatic hydrocarbon group. It is preferable that the aliphatic hydrocarbon group is saturated.

The aromatic hydrocarbon group as Yb⁰ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as Yb⁰ include benzene, fluorene, naphthalene, anthracene, phenanthrene, biphenyl, and an aromatic heterocyclic ring in which some carbon atoms constituting these aromatic ring are substituted with heteroatoms. Examples of the heteroatom in the aromatic heterocyclic rings include an oxygen atom, a sulfur atom, and a nitrogen atom.

Specific examples of the aromatic hydrocarbon group as Yb⁰ include a group obtained by removing one hydrogen atom from the aromatic ring (an aryl group such as a phenyl group or a naphthyl group) and a group in which one hydrogen atom of the aromatic ring is substituted with an alkylene group (for example, a benzyl group, a phenethyl group, or a 1-naphthylmethyl group). The number of carbon atoms in the alkylene group (alkyl chain in the arylalkyl group) is preferably in a range of 1 to 4, more preferably 1 or 2, and particularly preferably 1.

Examples of the cyclic aliphatic hydrocarbon group as Yb⁰ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The number of carbon atoms in the alicyclic hydrocarbon group is preferably in a range of 3 to 20 and more preferably in a range of 3 to 12.

The alicyclic hydrocarbon group may be a polycyclic group or a monocyclic group. As the monocyclic alicyclic hydrocarbon group, a group in which one or more hydrogen atoms have been removed from a monocycloalkane is preferable. The number of carbon atoms in the monocycloalkane is preferably 3 to 6, and specifically, cyclopentane and cyclohexane are exemplary examples. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, bornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

As the cyclic aliphatic hydrocarbon group as Yb⁰, a group in which one or more hydrogen atoms have been removed from a monocycloalkane or a polycycloalkane is preferable, a group in which one hydrogen atom has been removed from a polycycloalkane is more preferable, an adamantyl group, a norbornyl group, or a bornyl group is still more preferable, and an adamantyl group is particularly preferable.

The linear aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and most preferably 1 to 3 carbon atoms. As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to the alicyclic hydrocarbon group has preferably 2 to 10 carbon atoms, more preferably 3 to 6 carbon atoms, still more preferably 3 or 4 carbon atoms, and most preferably 3 carbon atoms. As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as Yb⁰ may have a heteroatom such as a heterocyclic ring. Specific examples thereof include lactone-containing cyclic groups each represented by General Formulae (a2-r-1) to (a2-r-7), —SO₂-containing cyclic groups each represented by General Formulae (a5-r-1) to (a5-r-4), and heterocyclic groups each represented by Chemical Formulae (r-hr-1) to (r-hr-16). In the formula, * represents a bonding site that is bonded to L⁰ in Formula (b0).

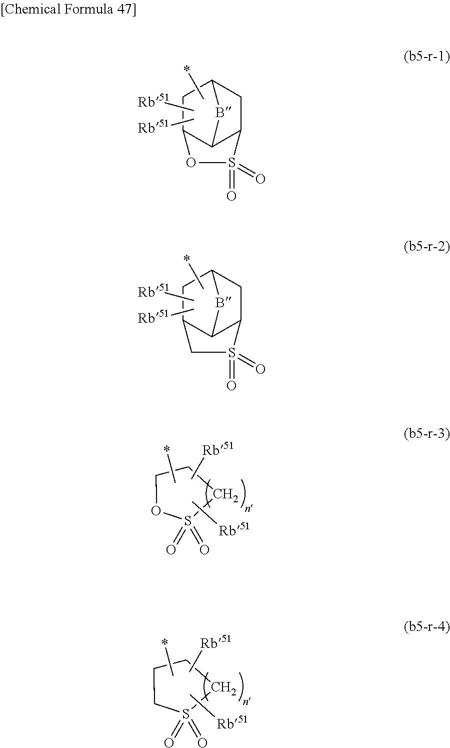

[In the formulae, Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group; R″ represents a hydrogen atom, an alkyl group, a lactone-containing cyclic group, or a —SO₂-containing cyclic group; B″ represents an oxygen atom, a sulfur atom, or an alkylene group having 1 to 5 carbon atoms, which may have an oxygen atom or a sulfur atom; and n′ represents an integer of 0 to 2. * represents a bonding site.]

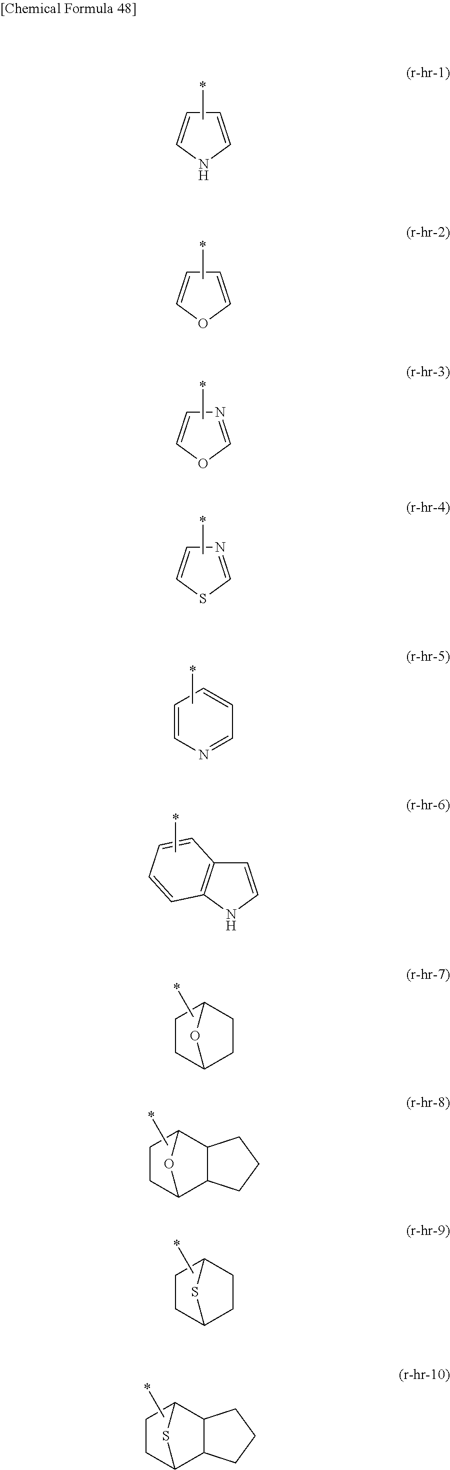

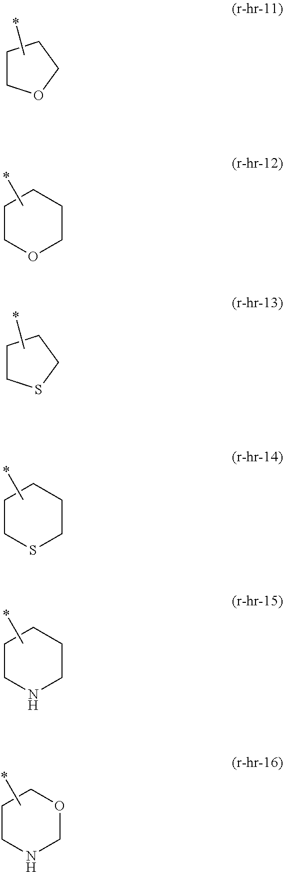

In General Formulae (b5-r-1) and (b5-r-2), B″ represents an alkylene group having 1 to 5 carbon atoms which may have an oxygen atom or a sulfur atom, an oxygen atom, or a sulfur atom.

B″ represents preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s each independently represent a hydrogen atom, an alkyl group, an alkoxy group, a halogen atom, a halogenated alkyl group, a hydroxyl group, —COOR″, —OC(═O)R″, a hydroxyalkyl group, or a cyano group. Among the examples, it is preferable that Rb′⁵¹'s each independently represent a hydrogen atom or a cyano group.

Specific examples of the groups each represented by General Formulae (b5-r-1) to (b5-r-4) are shown below. In the formulae shown below, “Ac” represents an acetyl group.

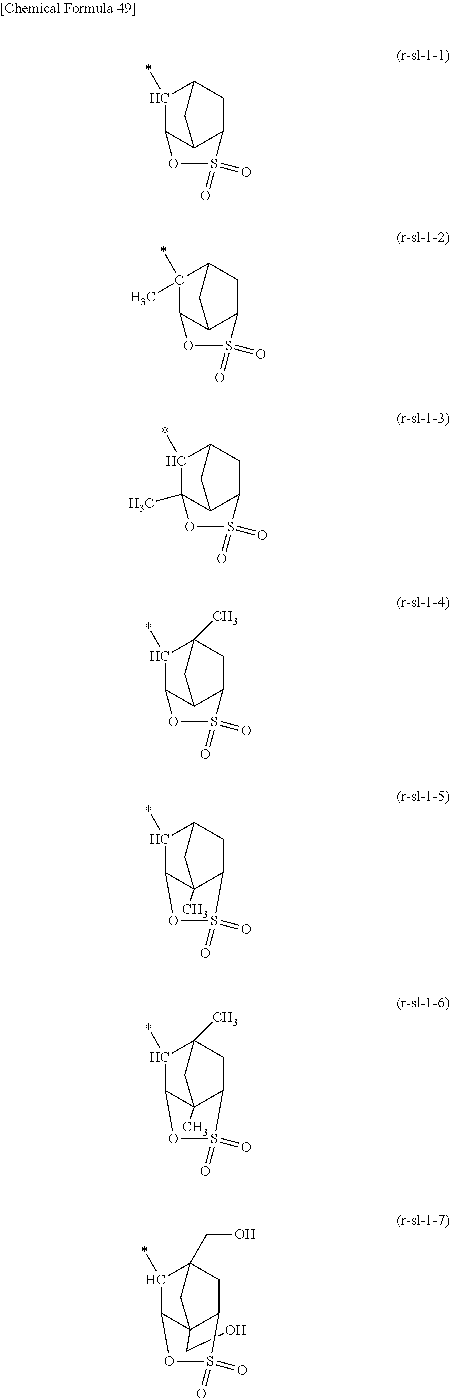

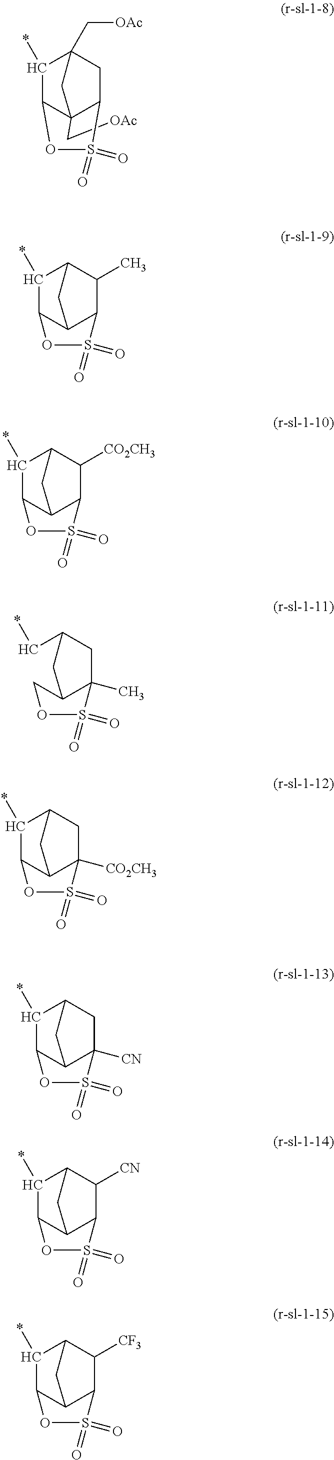

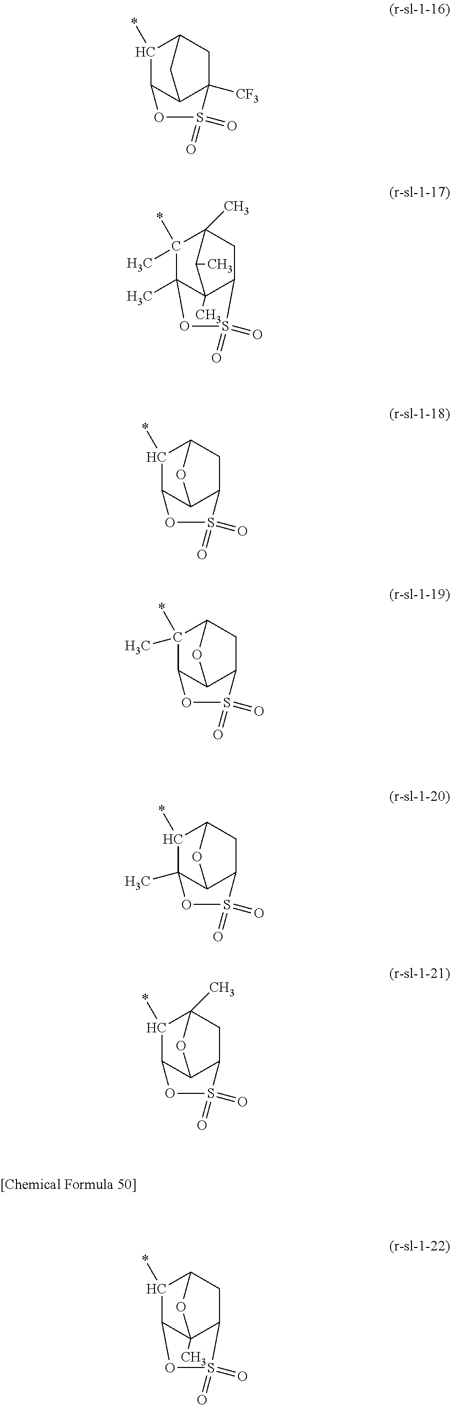

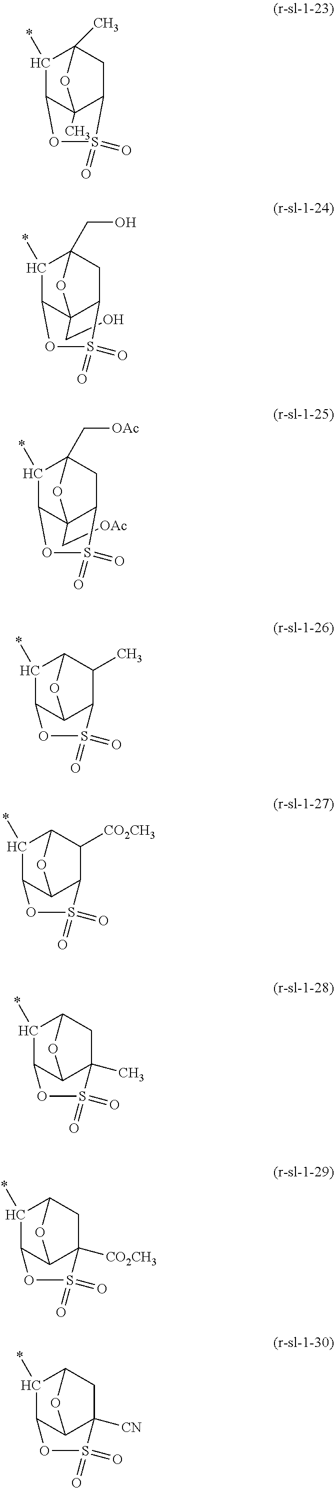

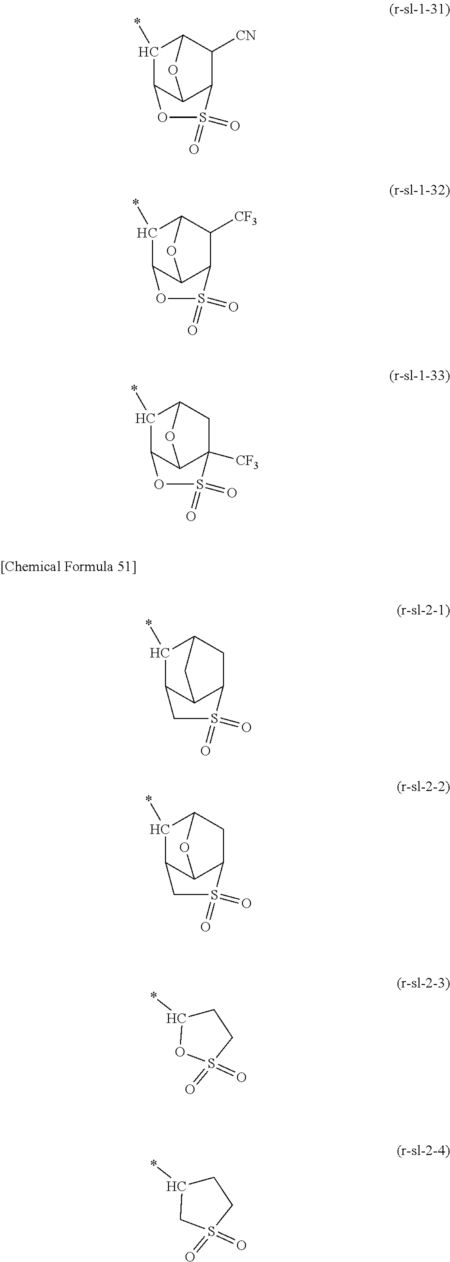

The cyclic group as Yb⁰ may have a substituent. Examples of the substituent include an alkyl group, an alkoxy group, a halogen atom, a halogenated alkyl group, a hydroxyl group, a carbonyl group, and a nitro group.

An alkyl group having 1 to 5 carbon atoms is preferable as the alkyl group serving as a substituent.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

A fluorine atom, a bromine atom, or an iodine atom is preferable as the halogen atom serving as a substituent.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group are substituted with the above-described halogen atoms.

The carbonyl group as the substituent is a group that substitutes a methylene group (—CH₂—) constituting the cyclic hydrocarbon group.

The cyclic hydrocarbon group as Yb⁰ may be a condensed cyclic group having a condensed ring in which an aliphatic hydrocarbon ring and an aromatic ring are condensed. Examples of the condensed ring include those obtained by fusing one or more aromatic rings with a polycycloalkane having a crosslinked ring-based polycyclic skeleton. Specific examples of the crosslinked ring polycycloalkane include a bicycloalkane such as bicyclo[2.2.1]heptane (norbornane) and bicyclo[2.2.2]octane. As the condensed cyclic group, a group having a condensed ring in which two or three aromatic rings are condensed with a bicycloalkane is preferable, and a group having a condensed ring in which two or three aromatic rings are condensed with bicyclo[2.2.2]octane is more preferable. Specific examples of the condensed cyclic group as Yb⁰ include groups represented by Formulae (r-br-1) and (r-br-2). In the formula, * represents a bonding site that is bonded to L⁰ in Formula (b0). Rx⁰ represents a substituent.

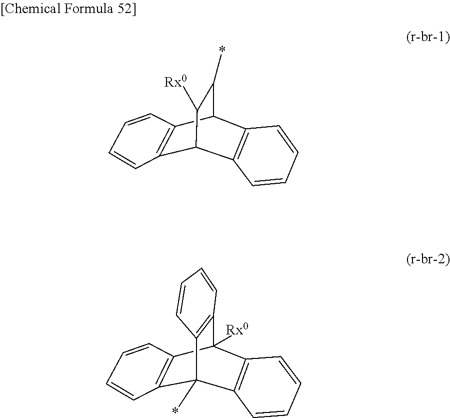

Examples of the substituent that the condensed cyclic group as Yb⁰ may have include an alkyl group, an alkoxy group, a halogen atom, a halogenated alkyl group, a hydroxyl group, a carbonyl group, a nitro group, an aromatic hydrocarbon group, and an alicyclic hydrocarbon group.

Examples of the alkyl group, the alkoxy group, the halogen atom, and the halogenated alkyl group as the substituent of the condensed cyclic group include the same groups as those for the substituent of the cyclic group.

Examples of the aromatic hydrocarbon group as the substituent of the condensed cyclic group include a group in which one hydrogen atom has been removed from the aromatic ring (an aryl group such as a phenyl group or a naphthyl group), a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group), and a heterocyclic group represented by any of Formulae (r-hr-1) to (r-hr-6).

Examples of the alicyclic hydrocarbon group as the substituent of the condensed cyclic group include a group in which one hydrogen atom has been removed from a monocycloalkane such as cyclopentane or cyclohexane, a group in which one hydrogen atom has been removed from a polycycloalkane such as adamantane, norbornane, isobornane, tricyclo[5.2.1.0^{2,6}]decane, or tetracyclododecane, a lactone-containing cyclic group represented by any of General Formulae (a2-r-1) to (a2-r-7), a —SO₂-containing cyclic group represented by any of General Formulae (a5-r-1) to (a5-r-4), and a heterocyclic group represented by any of Formulae (r-hr-7) to (r-hr-16).

The substituent of the condensed cyclic group may be an alkyl group in which some hydrogen atoms are substituted with heteroatom-containing groups. The alkyl group as a substituent of the condensed cyclic group may be, for example, a group in which some hydrogen atoms are substituted with heteroatom-containing groups. Alternatively, in the alkyl group as a substituent of the condensed cyclic group, some carbon atoms (methylene group and the like) constituting a hydrocarbon chain may be substituted with heteroatom-containing groups. Examples of the heteroatom include an oxygen atom, a sulfur atom, and a nitrogen atom.

Examples of the heteroatom-containing group that substitutes a hydrogen atom of an alkyl group include a hydroxyl group, an alkoxy group, a halogen atom, a halogenated alkyl group, and a carboxy group.

Examples of the heteroatom-containing group that substitutes a carbon atom constituting a hydrocarbon chain of an alkyl group include —O—, —C(═O)—O—, —O—C(═O)—, —C(═O)—, —O—C(═O)—O—, —C(═O)—NH—, —NH—, —S—, —S(═O)₂—, and —S(═O)₂—O—. As the heteroatom-containing group, —O—, —C(═O)—O—, —O—C(═O)—, or —S(═O)₂—O— is preferable, and —C(═O)—O— is more preferable.

Examples of the substituent of the condensed cyclic group include a group represented by —C(═O)—O—Rx⁰¹. Rx⁰¹ represents an alkyl group, and may be linear, branched, or cyclic. Examples of the linear alkyl group include an alkyl group having 1 to 5 carbon atoms. Among these, a methyl group, an ethyl group, an n-propyl group, or an n-butyl group is preferable. Examples of the branched alkyl group include an alkyl group having 3 to 5 carbon atoms. Among these, an isopropyl group, a tert-butyl group, a sec-butyl group, or an isobutyl group is preferable. Examples of the cyclic alkyl group include a group obtained by removing one hydrogen atom from a cycloalkane having 1 to 8 carbon atoms. Among these, a cyclopropyl group, a cyclobutyl group, a cyclopentyl group, or a cyclohexyl group is preferable, and a cyclopentyl group or a cyclohexyl group is more preferable.

It is preferable that Yb⁰ represents an adamantyl group which may have a substituent, a cyclic group having a steroid skeleton which may have a substituent, a lactone-containing cyclic group represented by any of Formulae (a2-r-1) to (a2-r-7), a —SO₂-containing cyclic group represented by any of Formulae (a5-r-1) to (a5-r-4), a condensed cyclic group having a condensed ring in which an aliphatic hydrocarbon ring and an aromatic ring are condensed, or an aromatic hydrocarbon group which may have a substituent. A group represented by Formula (r-br-1) or (r-br-2) is preferable as the condensed cyclic group. Examples of the substituent of the adamantyl group include a hydroxy group and a carbonyl group.

Examples of L⁰ in a case where L⁰-Yb⁰ is not-O—C(═O)—Yb⁰ include a divalent linking group having —C(═O)—O—, a divalent linking group having —S(═O)₂—O—, and a group represented by Formula (L0-3) (here, a case where nb03 represents 1 and both Lb⁰³¹ and Lb⁰³² represent a single bond is excluded). Examples of the divalent linking group having —C(═O)—O— include a group represented by Formula (L01-1), and a group represented by Formula (L0-1-1), (L0-1-2), or (L0-1-5) is preferable. Examples of the divalent linking group having —S(═O)₂—O— include a group represented by Formula (L0-2), and a group represented by (L0-1-4) or (L0-1-5) is preferable.

Case where L⁰-Yb⁰ represents —O—C(═O)—Yb⁰:

In this case, Yb⁰ represents an alicyclic group which may have a substituent, a condensed cyclic group of an aliphatic ring and an aromatic ring, which may have a substituent, or an aromatic hydrocarbon group which has a substituent. In the aromatic hydrocarbon group, at least one hydrogen atom of the aromatic ring is substituted with an alkyl group or an alkoxy group.

Examples of the alicyclic group which may have a substituent as Yb⁰ include the same groups as those in the section of “Case where L⁰-Yb⁰ does not represent —O—C(═O)—Yb⁰”. Examples of the alicyclic group include a group obtained by removing one or more hydrogen atoms from a monocycloalkane or a polycycloalkane, a cyclic group having a steroid skeleton, a lactone-containing cyclic group represented by any of Formulae (a2-r-1) to (a2-r-7), a —SO₂-containing cyclic group represented by any of Formulae (a5-r-1) to (a5-r-4), and a heterocyclic group represented by any of Chemical Formulae (r-hr-1) to (r-hr-16).

Examples of the substituent contained in the alicyclic group as Yb⁰ include the same groups as those in the section of “Case where L⁰-Yb⁰ does not represent —O—C(═O)—Yb⁰”.

Examples of the condensed cyclic group of an aliphatic ring and an aromatic ring as Yb⁰ include the same groups as those in the section of “Case where L⁰-Yb⁰ does not represent —O—C(═O)—Yb⁰”. Examples of the condensed cyclic group include a group represented by Formula (r-br-1) or (r-br-2).

Examples of the substituent contained in the condensed cyclic group as Yb⁰ include the same groups as those in the section of “Case where L⁰-Yb⁰ does not represent —O—C(═O)—Yb⁰”.

Examples of the aromatic hydrocarbon group having a substituent as Yb⁰ include the same groups as those in the section of “Case where L⁰-Yb⁰ does not represent —O—C(═O)—Yb⁰”. Here, the aromatic hydrocarbon group may be formed such that at least one hydrogen atom of the aromatic ring is substituted with an alkyl group or an alkoxy group. The aromatic ring may have an alkyl group or an alkoxy group or may have both an alkyl group and an alkoxy group. It is preferable that the aromatic ring has an alkoxy group.

The aromatic ring may have a substituent other than the alkyl group and the alkoxy group. Examples of the substituent include a halogen atom, a halogenated alkyl group, a hydroxyl group, a carbonyl group, and a nitro group. Among these, the aromatic ring has preferably a halogen atom as a substituent and more preferably an iodine atom as a substituent.

The aromatic ring contained in the aromatic hydrocarbon group as Yb⁰ is preferably a benzene ring or a naphthalene ring and more preferably a benzene ring.

In a case where L⁰-Yb⁰ represents —O—C(═O)—Yb⁰, Formula (b0) can be represented by Formula (b02-1).

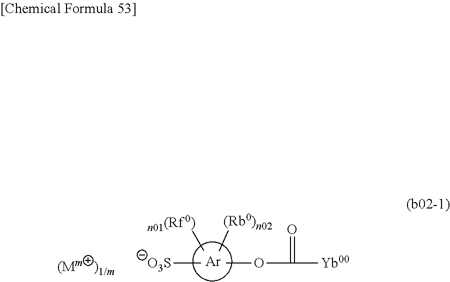

[In the formula, Ar, Rf⁰, n01, and n02 each have the same definition as that for Ar, Rf⁰, n01, and n02 in Formula (b0). Yb⁰⁰ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of the aromatic ring is substituted with an alkyl group or an alkoxy group. m represents an integer of 1 or greater, and M^{m+} represents an m-valent cation.]

A compound represented by General Formula (b0-1) is preferable as the component (B0).

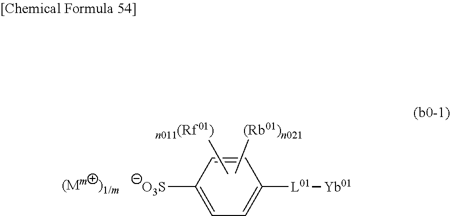

[In the formula, Rf⁰¹ represents a fluorinated alkyl group having 1 to 5 carbon atoms or a fluorine atom. L⁰¹ represents a divalent linking group having —C(═O)—O—, —O—C(═O)—, or —O—S(═O)₂—. Yb⁰¹ represents a cyclic group. Rb⁰¹ represents an organic group. n011 represents an integer of 1 or greater, n021 represents an integer of 0 or greater, and n011+n021≤4 is satisfied. Here, in a case where L⁰¹-Yb⁰¹ represents —O—C(═O)—Yb⁰¹, Yb⁰¹ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of the aromatic ring is substituted with an alkyl group or an alkoxy group. In a case where n011 represents 2 or greater, a plurality of Rf⁰¹'s may be the same as or different from each other. In a case where n021 represents 2 or greater, a plurality of Rb⁰¹'s may be the same as or different from each other. In a case where n031 represents 2, two L⁰¹'s may be the same as or different from each other. m represents an integer of 1 or greater, and M^{m+} represents an m-valent cation.]

In Formula (b0-1), Rf⁰¹, Rb⁰¹, L⁰¹, and Yb⁰¹ each have the same definition as that for Rf⁰, Rb⁰, L⁰, and Yb⁰ in Formula (b0). n011 is preferably an integer of 1 to 4, more preferably an integer of 2 to 4, still more preferably 3 or 4, and particularly preferably 4. n021 represents preferably an integer of 0 to 3, more preferably an integer of 0 to 2, still more preferably 0 or 1, and particularly preferably 0.

A compound represented by General Formula (b0-1-1) is more preferable as the component (B0).

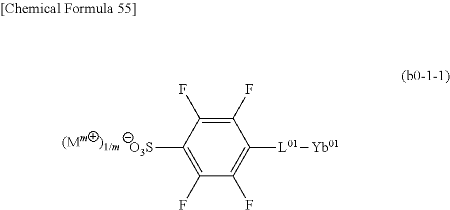

[In the formula, L⁰¹ and Yb⁰¹ each have the same definition as that for L⁰¹ and Yb⁰¹ in Formula (b0-1). m represents an integer of 1 or greater, and M^{m+} represents an m-valent organic cation.]

Specific examples of the component (B0) are shown below but are not limited thereto. In the following formulae, m represents an integer of 1 or greater, and M^{m+} represents an m-valent cation.

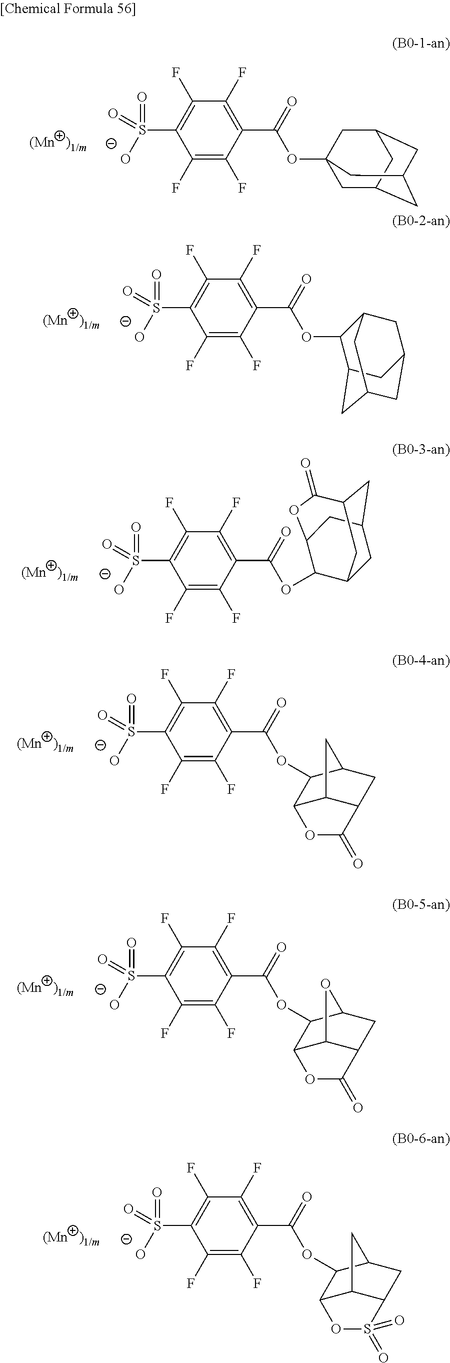

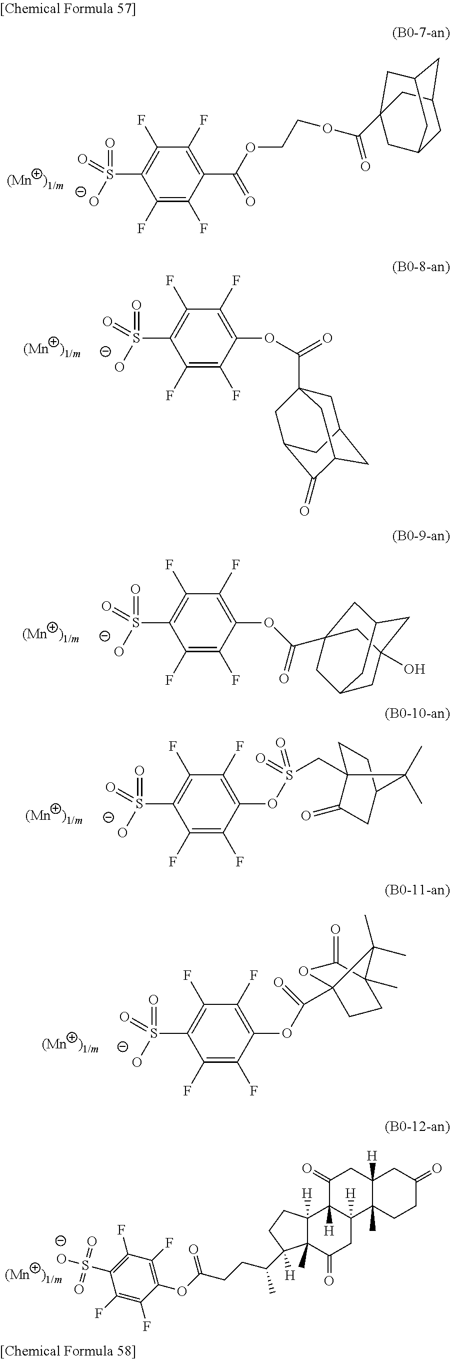

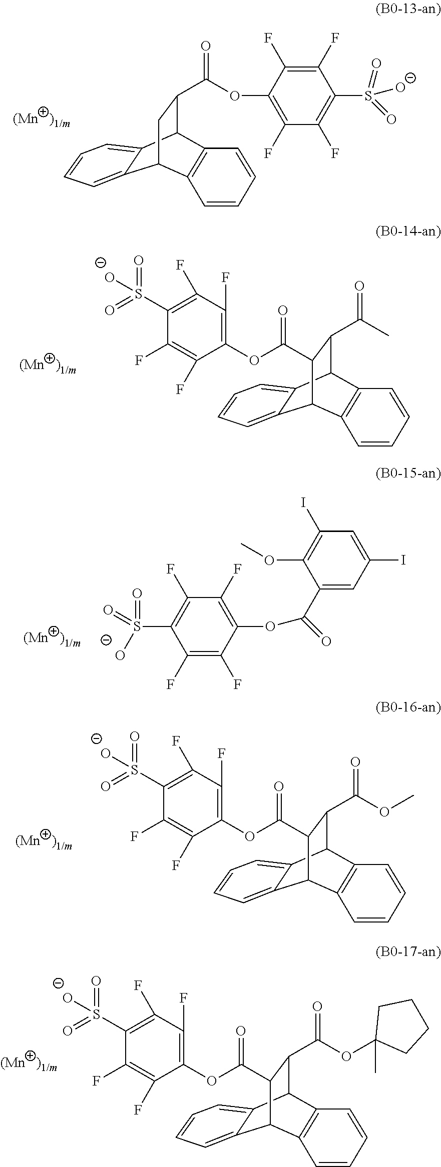 [SMILES: O=C(Oc1c(F)c(F)c(S(=O)(=O)[O-])c(F)c1F)C1CC2c3ccccc3C1c1ccccc12 ; CC(=O)C1C2c3ccccc3C(c3ccccc32)C1C(=O)Oc1c(F)c(F)c(S(=O)(=O)[O-])c(F)c1F ; COc1c(I)cc(I)cc1C(=O)Oc1c(F)c(F)c(S(=O)(=O)[O-])c(F)c1F ; COC(=O)C1C2c3ccccc3C(c3ccccc32)C1C(=O)Oc1c(F)c(F)c(S(=O)(=O)[O-])c(F)c1F ; CC1(OC(=O)C2C3c4ccccc4C(c4ccccc43)C2C(=O)Oc2c(F)c(F)c(S(=O)(=O)[O-])c(F)c2F)CCCC1]

### {Cation Moiety}

In Formula (b0), M^{m+} represents an m-valent onium cation. The onium cation is preferably a sulfonium cation or an iodonium cation. m represents an integer of 1 or greater.

Preferred examples of the cation moiety ((M^{m+})_{l/m}) include organic cations each represented by General Formulae (ca-1) to (ca-3).

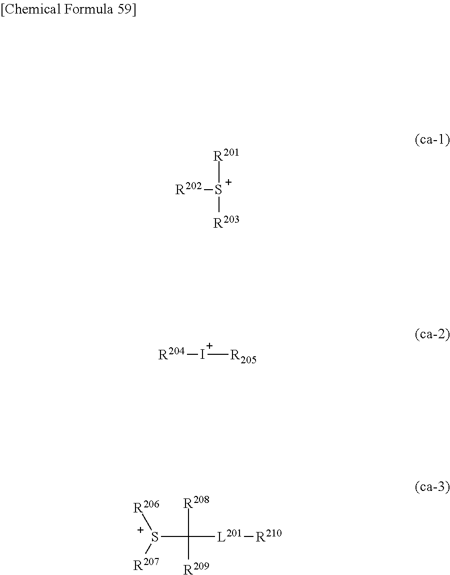

[In the formulae, R²⁰¹ to R²⁰⁷ each independently represent an aryl group, an alkyl group, or an alkenyl group, each of which may have a substituent. R²⁰¹ to R²⁰³ and R²⁰⁶ and R²⁰⁷ may be bonded to each other to form a ring together with the sulfur atoms in the formulae. R²⁰⁸ and R²⁰⁹ each independently represent a hydrogen atom or an alkyl group having 1 to 5 carbon atoms. R²¹⁰ represents an aryl group which may have a substituent, an alkyl group which may have a substituent, an alkenyl group which may have a substituent, or a —SO₂-containing cyclic group which may have a substituent. L²⁰¹ represents —C(═O)— or —C(═O)—O—.]

In General Formulae (ca-1) to (ca-3), examples of the aryl group as R²⁰¹ to R²⁰⁷ include an unsubstituted aryl group having 6 to 20 carbon atoms, where a phenyl group or a naphthyl group is preferable.

The alkyl group as R²⁰¹ to R²⁰⁷ is preferably a chain-like or cyclic alkyl group which has 1 to 30 carbon atoms.

The alkenyl group as R²⁰¹ to R²⁰⁷ preferably has 2 to 10 carbon atoms.

Examples of the substituent that R²⁰¹ to R²⁰⁷, and R²¹⁰ include an alkyl group, a halogen atom, a halogenated alkyl group, a carbonyl group, a cyano group, an amino group, an aryl group, and a group represented by any of General Formulae (ca-r-1) to (ca-r-7).

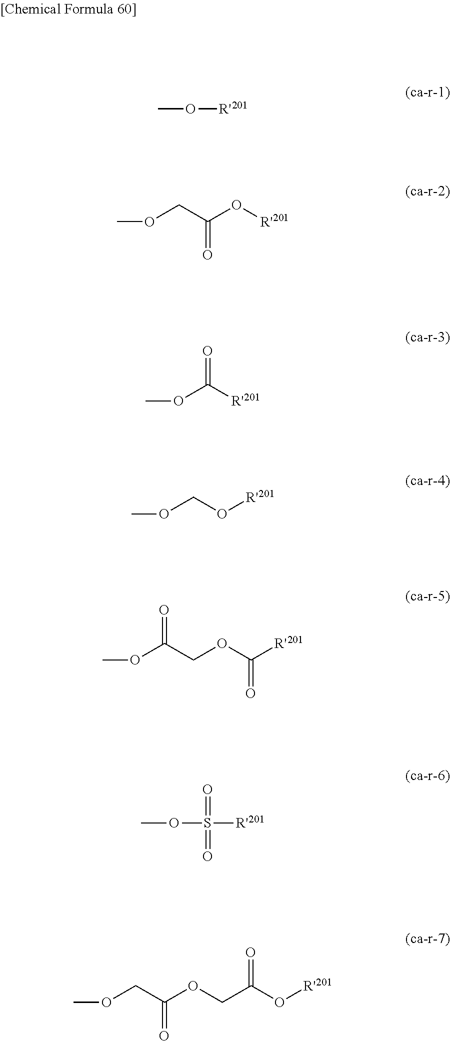

[In the formulae, R′²⁰¹'s each independently represents a hydrogen atom, a cyclic group which may have a substituent, a chain-like alkyl group which may have a substituent, or a chain-like alkenyl group which may have a substituent.]

Cyclic Group which May have Substituent:

The cyclic group is preferably a cyclic hydrocarbon group, and the cyclic hydrocarbon group may be an aromatic hydrocarbon group or an aliphatic hydrocarbon group. The aliphatic hydrocarbon group indicates a hydrocarbon group that has no aromaticity. Further, the aliphatic hydrocarbon group may be saturated or unsaturated. In general, it is preferable that the aliphatic hydrocarbon group is saturated.

The aromatic hydrocarbon group as R′²⁰¹ is a hydrocarbon group having an aromatic ring. The aromatic hydrocarbon group has preferably 3 to 30 carbon atoms, more preferably 5 to 30 carbon atoms, still more preferably 5 to 20 carbon atoms, particularly preferably 6 to 15 carbon atoms, and most preferably 6 to 10 carbon atoms. Here, the number of carbon atoms in a substituent is not included in the number of carbon atoms.

Specific examples of the aromatic ring contained in the aromatic hydrocarbon group as R′²⁰¹ include benzene, fluorene, naphthalene, anthracene, phenanthrene, biphenyl, or an aromatic heterocyclic ring in which some carbon atoms constituting any of these aromatic rings have been substituted with heteroatoms. Examples of the heteroatom in the aromatic heterocyclic rings include an oxygen atom, a sulfur atom, and a nitrogen atom.

Specific examples of the aromatic hydrocarbon group as R′²⁰¹ include a group in which one hydrogen atom has been removed from the aromatic ring (an aryl group such as a phenyl group or a naphthyl group), and a group in which one hydrogen atom in the aromatic ring has been substituted with an alkylene group (for example, an arylalkyl group such as a benzyl group, a phenethyl group, a 1-naphthylmethyl group, a 2-naphthylmethyl group, 1-naphthylethyl group, or a 2-naphthylethyl group). The alkylene group (alkyl chain in the arylalkyl group) has preferably 1 to 4 carbon atoms, more preferably 1 or 2 carbon atoms, and particularly preferably 1 carbon atom.

Examples of the cyclic aliphatic hydrocarbon group as R′²⁰¹ include an aliphatic hydrocarbon group having a ring in the structure thereof.

Examples of the aliphatic hydrocarbon group having a ring in the structure thereof include an alicyclic hydrocarbon group (group in which one hydrogen atom has been removed from an aliphatic hydrocarbon ring), a group in which the alicyclic hydrocarbon group is bonded to the terminal of a linear or branched aliphatic hydrocarbon group, and a group in which the alicyclic hydrocarbon group is interposed in the middle of a linear or branched aliphatic hydrocarbon group.

The alicyclic hydrocarbon group has preferably 3 to 20 carbon atoms and more preferably 3 to 12 carbon atoms.

The alicyclic hydrocarbon group may be a polycyclic group or a monocyclic group. As the monocyclic alicyclic hydrocarbon group, a group in which one or more hydrogen atoms have been removed from a monocycloalkane is preferable. The number of carbon atoms in the monocycloalkane is preferably 3 to 6, and specifically, cyclopentane and cyclohexane are exemplary examples. As the polycyclic alicyclic hydrocarbon group, a group in which one or more hydrogen atoms have been removed from a polycycloalkane is preferable, and the number of carbon atoms of the polycycloalkane is preferably in a range of 7 to 30. Among these, a polycycloalkane having a crosslinked ring polycyclic skeleton such as adamantane, norbornane, isobornane, tricyclo[5.2.1.0^{2,6}]decane, or tetracyclododecane; and a polycycloalkane having a condensed ring polycyclic skeleton such as a cyclic group having a steroid skeleton are preferable as the polycycloalkane.

Among these examples, as the cyclic aliphatic hydrocarbon group as R′²⁰¹, a group in which one or more hydrogen atoms have been removed from a monocycloalkane or a polycycloalkane is preferable, a group in which one hydrogen atom has been removed from a polycycloalkane is more preferable, an adamantyl group or a norbornyl group is particularly preferable, and an adamantyl group is most preferable.

The linear or branched aliphatic hydrocarbon group which may be bonded to the alicyclic hydrocarbon group has preferably 1 to 10 carbon atoms, more preferably 1 to 6 carbon atoms, still more preferably 1 to 4 carbon atoms, and particularly preferably 1 to 3 carbon atoms.

As the linear aliphatic hydrocarbon group, a linear alkylene group is preferable. Specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

As the branched aliphatic hydrocarbon group, a branched alkylene group is preferable. Specifically, alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂—, and —CH₂CH(CH₃)CH₂—; and alkyltetramethylene groups such as —CH(CH₃)CH₂CH₂CH₂— and —CH₂CH(CH₃)CH₂CH₂— are exemplary examples. As the alkyl group in the alkylalkylene group, a linear alkyl group having 1 to 5 carbon atoms is preferable.

Further, the cyclic hydrocarbon group as R′²⁰¹ may have a heteroatom such as a heterocyclic ring. Specific examples thereof include lactone-containing cyclic groups each represented by General Formulae (a2-r-1) to (a2-r-7), —SO₂-containing cyclic group each represented by General Formulae (a5-r-1) to (a5-r-4), and other heterocyclic groups each represented by Chemical Formulae (r-hr-1) to (r-hr-16).

Examples of the substituent for the cyclic group as R′²⁰¹ include an alkyl group, an alkoxy group, a halogen atom, a halogenated alkyl group, a hydroxyl group, a carbonyl group, and a nitro group.

As the alkyl group as the substituent, an alkyl group having 1 to 5 carbon atoms is preferable, and a methyl group, an ethyl group, a propyl group, an n-butyl group, or a tert-butyl group is most preferable.

As the alkoxy group as the substituent, an alkoxy group having 1 to 5 carbon atoms is preferable, a methoxy group, an ethoxy group, an n-propoxy group, an iso-propoxy group, an n-butoxy group, or a tert-butoxy group is more preferable, and a methoxy group or an ethoxy group is most preferable.

As the halogen atom as a substituent, a fluorine atom is preferable.

Example of the above-described halogenated alkyl group as the substituent includes a group in which some or all hydrogen atoms in an alkyl group having 1 to 5 carbon atoms such as a methyl group, an ethyl group, a propyl group, an n-butyl group, or a tert-butyl group have been substituted with the above-described halogen atoms.

The carbonyl group as the substituent is a group that substitutes a methylene group (—CH₂—) constituting the cyclic hydrocarbon group.

Chain-Like Alkyl Group which May have Substituent:

The chain-like alkyl group as R′²⁰¹ may be linear or branched.

The linear alkyl group has preferably 1 to 20 carbon atoms, more preferably 1 to 15 carbon atoms, and most preferably 1 to 10 carbon atoms.

The branched alkyl group has preferably 3 to 20 carbon atoms, more preferably 3 to 15 carbon atoms, and most preferably 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain-Like Alkenyl Group which May have Substituent:

The chain-like alkenyl group as R′²⁰¹ may be linear or branched, and has preferably 2 to 10 carbon atoms, more preferably 2 to 5 carbon atoms, still more preferably 2 to 4 carbon atoms, and particularly preferably 3 carbon atoms. Examples of the linear alkenyl group include a vinyl group, a propenyl group (allyl group), and a butenyl group. Examples of the branched alkenyl group include a 1-methylvinyl group, a 2-methylvinyl group, a 1-methylpropenyl group, and a 2-methylpropenyl group.

Among the examples, as the chain-like alkenyl group, a linear alkenyl group is preferable, a vinyl group or a propenyl group is more preferable, and a vinyl group is particularly preferable.

Examples of the substituent for the chain-like alkyl group or alkenyl group as R′²⁰¹ include an alkoxy group, a halogen atom, a halogenated alkyl group, a hydroxyl group, a carbonyl group, a nitro group, an amino group, and a cyclic group as R′²⁰¹.

Examples of the cyclic group which may have a substituent, the chain-like alkyl group which may have a substituent, and the chain-like alkenyl group which may have a substituent as R′²⁰¹ include those for the acid dissociable group represented by Formula (a1-r-2) which are the exemplary examples of the cyclic group which may have a substituent and the chain-like alkyl group which may have a substituent, in addition to those described above.

Among the examples, R′²⁰¹ represents preferably a cyclic group which may have a substituent and more preferably a cyclic hydrocarbon group which may have a substituent. More specific preferred examples thereof include a phenyl group, a naphthyl group, a group in which one or more hydrogen atoms have been removed from a polycycloalkane, a lactone-containing cyclic group represented by any of General Formulae (a2-r-1) to (a2-r-7), and a —SO₂-containing cyclic group represented by any of General Formulae (a5-r-1) to (a5-r-4).

In General Formulae (ca-1) to (ca-5), in a case where R²⁰¹ to R²⁰³, R²⁰⁶ and R²⁰⁷ and R²¹¹ and R²¹² are bonded to each other to form a ring with a sulfur atom in the formula, these groups may be bonded to one another via a heteroatom such as a sulfur atom, an oxygen atom, or a nitrogen atom, or a functional group such as a carbonyl group, —SO—, —SO₂—, —SO₃—, —COO—, —CONH— or —N(R_{N})— (here, R_{N} represents an alkyl group having 1 to 5 carbon atoms). Regarding the ring to be formed, it is preferable that a ring containing the sulfur atom in the formula in the ring skeleton thereof is a 3-membered to 10-membered ring and it is particularly preferable that it is a 5-membered to 7-membered ring, in a case where the sulfur atom is included. Specific examples of the ring to be formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ each independently represent a hydrogen atom or an alkyl group having 1 to 5 carbon atoms and preferably a hydrogen atom or an alkyl group having 1 to 3 carbon atoms. In a case where R²⁰⁸ and R²⁰⁹ each represent an alkyl group, R²⁰⁸ and R²⁰⁹ may be bonded to each other to form a ring.

R²¹⁰ represents an aryl group which may have a substituent, an alkyl group which may have a substituent, an alkenyl group which may have a substituent, or a —SO₂-containing cyclic group which may have a substituent.

Examples of the aryl group as R²¹⁰ include an unsubstituted aryl group having 6 to 20 carbon atoms. Among these, a phenyl group or a naphthyl group is preferable.

As the alkyl group represented by R²¹⁰, a chain-like or cyclic alkyl group having 1 to 30 carbon atoms is preferable.

It is preferable that the alkenyl group as R²¹⁰ has 2 to 10 carbon atoms.

As the —SO₂-containing cyclic group as R²¹⁰ which may have a substituent, the “—SO₂-containing polycyclic group” is preferable, and a group represented by General Formula (a5-r-1) is more preferable.

Specific suitable examples of the cation represented by Formula (ca-1) include cations each represented by Chemical Formulae (ca-1-1) to (ca-1-75).

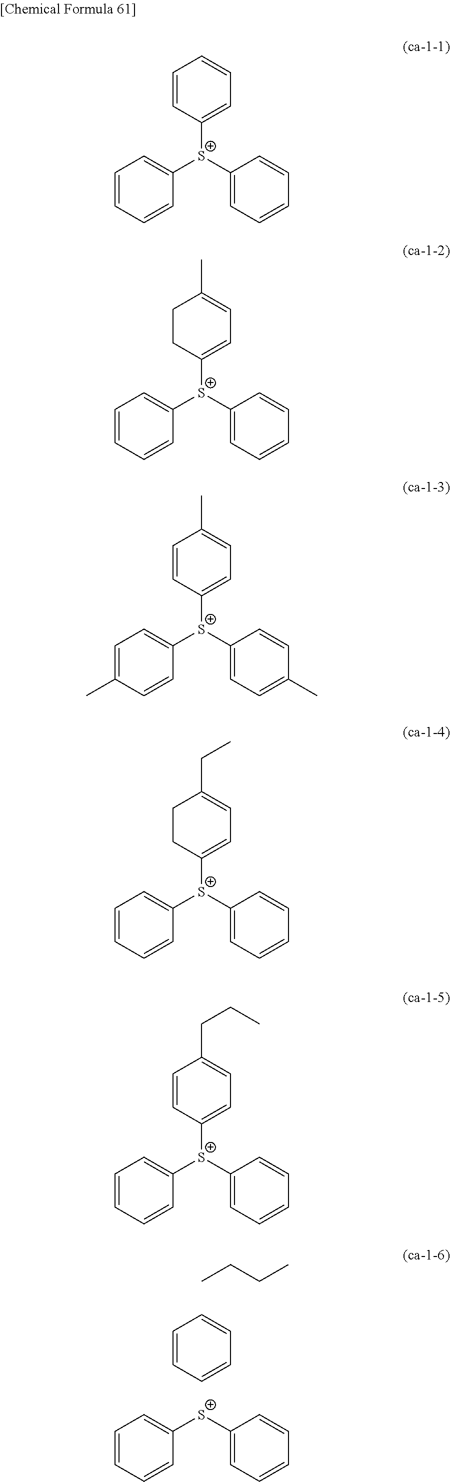

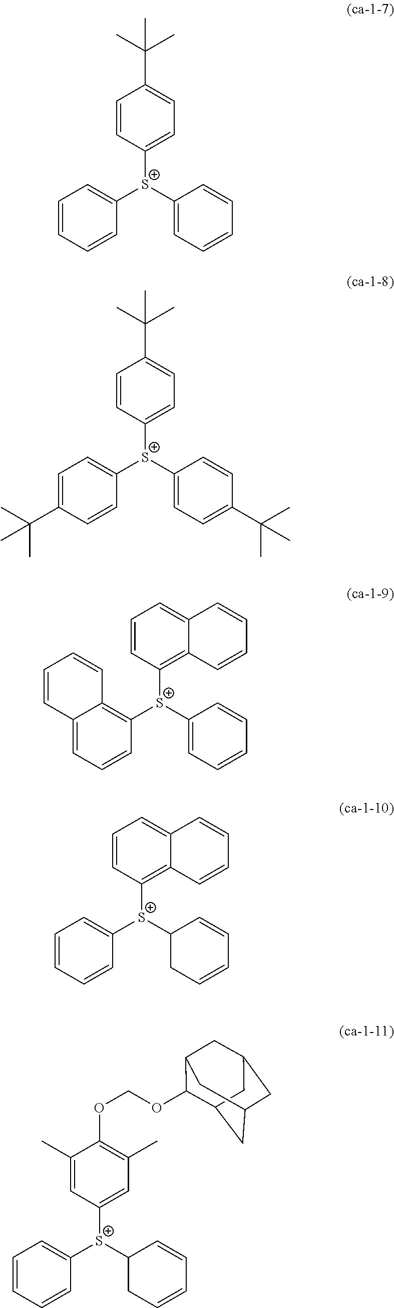 [SMILES: CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; C1=CCC([S+](c2ccccc2)c2cccc3ccccc23)C=C1 ; Cc1cc([S+](c2ccccc2)C2C=CC=CC2)cc(C)c1OCOC1C2CC3CC(C2)CC1C3]

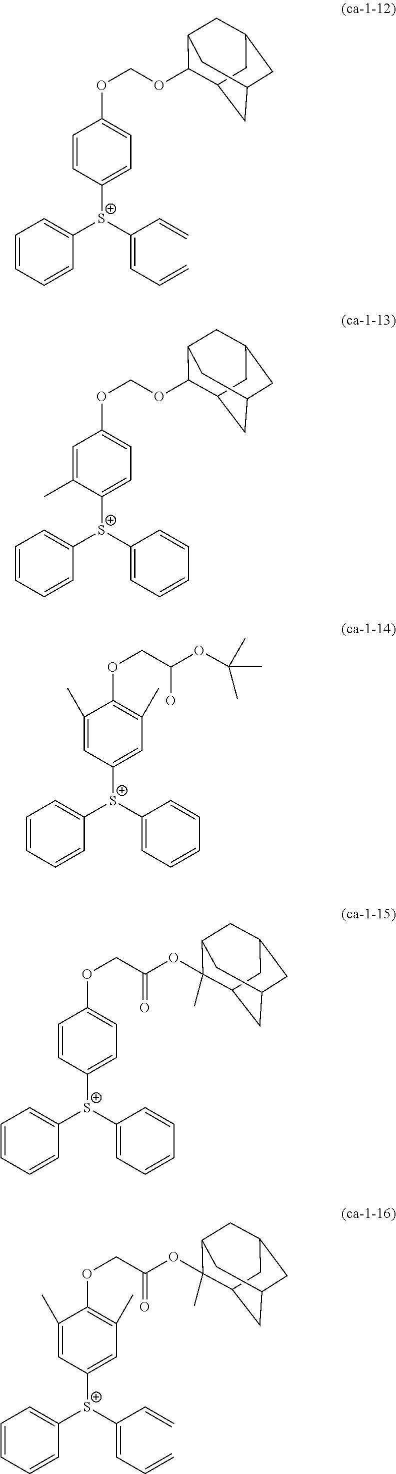 [SMILES: C=C/C=C(\C=C)[S+](c1ccccc1)c1ccc(OCOC2C3CC4CC(C3)CC2C4)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(O)OC(C)(C)C ; CC1(OC(=O)COc2ccc([S+](c3ccccc3)c3ccccc3)cc2)C2CC3CC(C2)CC1C3 ; C=C/C=C(\C=C)[S+](c1ccccc1)c1cc(C)c(OCC(=O)OC2(C)C3CC4CC(C3)CC2C4)c(C)c1]

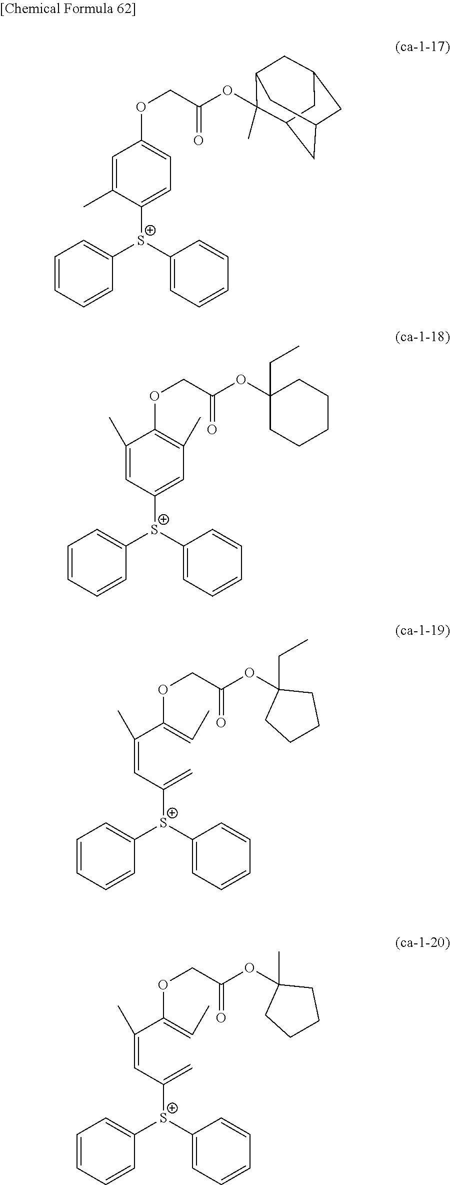 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; C=C(/C=C(C)\C(=C\C)OCC(=O)OC1(CC)CCCC1)[S+](c1ccccc1)c1ccccc1 ; C=C(/C=C(C)\C(=C\C)OCC(=O)OC1(C)CCCC1)[S+](c1ccccc1)c1ccccc1]

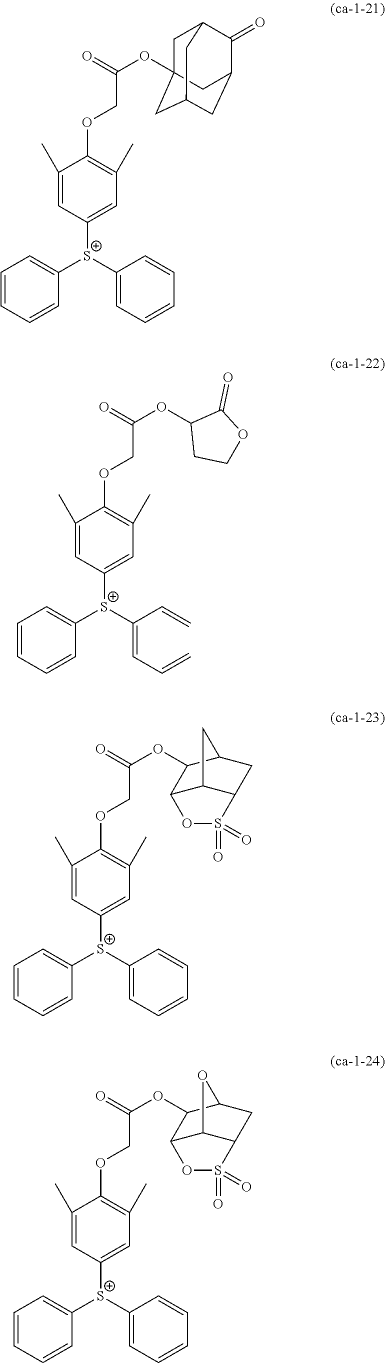 [SMILES: Cc1cc([S+](c2ccccc2)c2ccccc2)cc(C)c1OCC(=O)OC12CC3CC(C1)C(=O)C(C3)C2 ; C=C/C=C(\C=C)[S+](c1ccccc1)c1cc(C)c(OCC(=O)OC2CCOC2=O)c(C)c1 ; Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O]

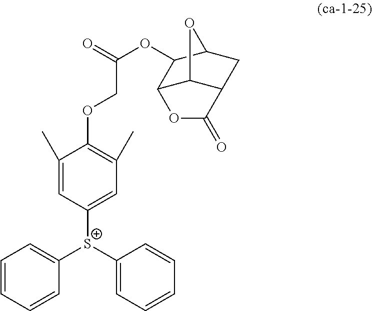 [SMILES: Cc1cc([S+](c2ccccc2)c2ccccc2)cc(C)c1OCC(=O)OC1C2CC3C(=O)OC1C3O2]

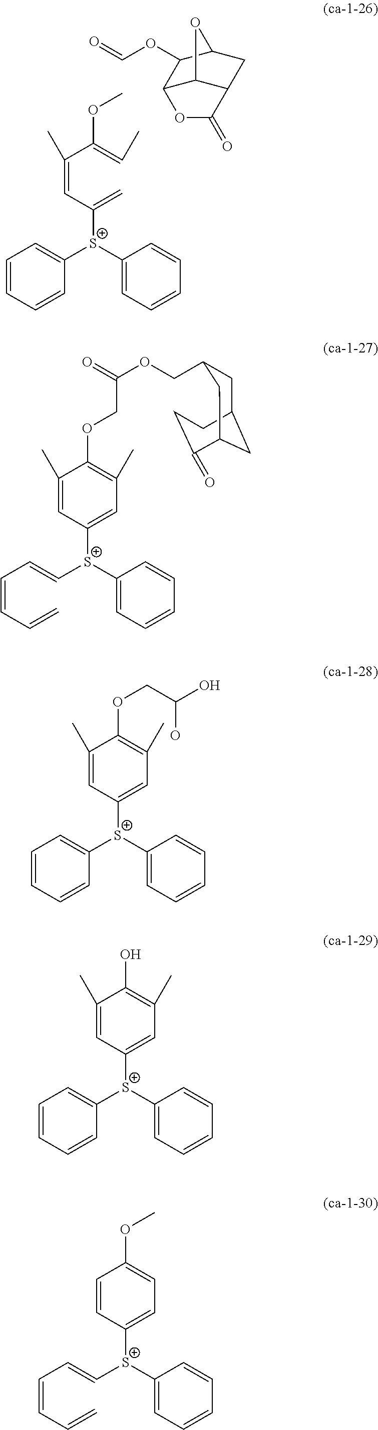 [SMILES: C=C(/C=C(C)\C(=C\C)OC)[S+](c1ccccc1)c1ccccc1 ; O=COC1C2CC3C(=O)OC1C3O2 ; C=C/C=C\C=C\[S+](c1ccccc1)c1cc(C)c(OCC(=O)OCC2CC3CCC(=O)C(C3)C2)c(C)c1 ; Cc1cc([S+](c2ccccc2)c2ccccc2)cc(C)c1OCC(O)O ; Cc1cc([S+](c2ccccc2)c2ccccc2)cc(C)c1O ; C=C/C=C\C=C\[S+](c1ccccc1)c1ccc(OC)cc1]

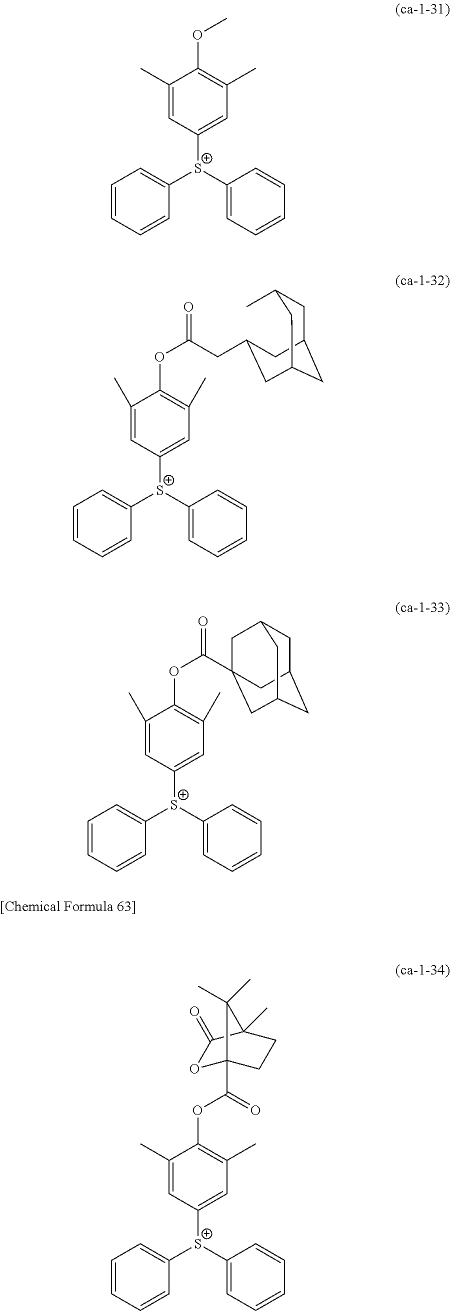 [SMILES: COc1c(C)cc([S+](c2ccccc2)c2ccccc2)cc1C ; Cc1cc([S+](c2ccccc2)c2ccccc2)cc(C)c1OC(=O)CC1CC2CC(C)CC(C1)C2 ; Cc1cc([S+](c2ccccc2)c2ccccc2)cc(C)c1OC(=O)C12CC3CC(CC(C3)C1)C2 ; Cc1cc([S+](c2ccccc2)c2ccccc2)cc(C)c1OC(=O)C12CCC(C)(C(=O)O1)C2(C)C]

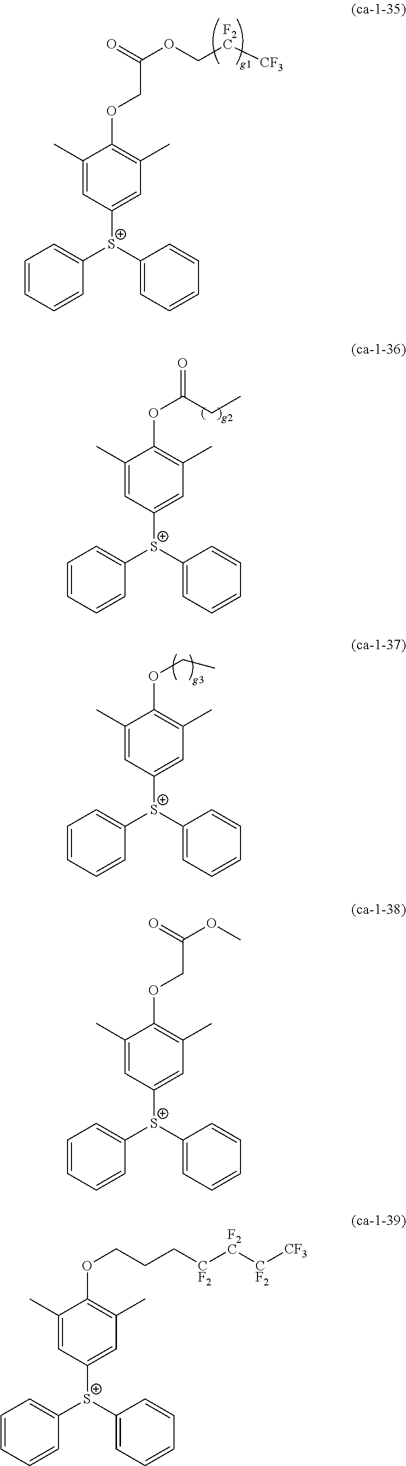

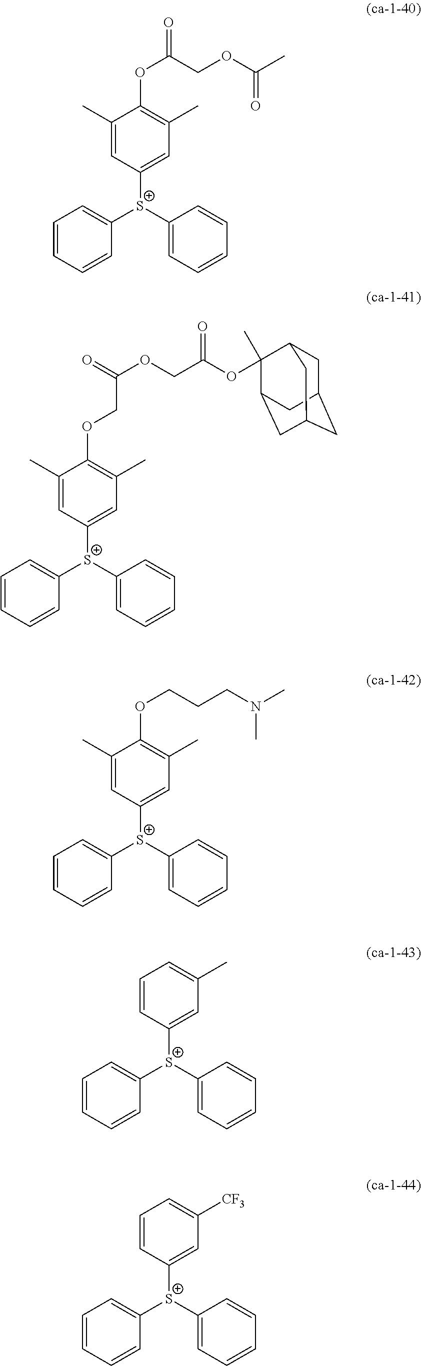 [SMILES: CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1 ; FC(F)(F)c1cccc([S+](c2ccccc2)c2ccccc2)c1]

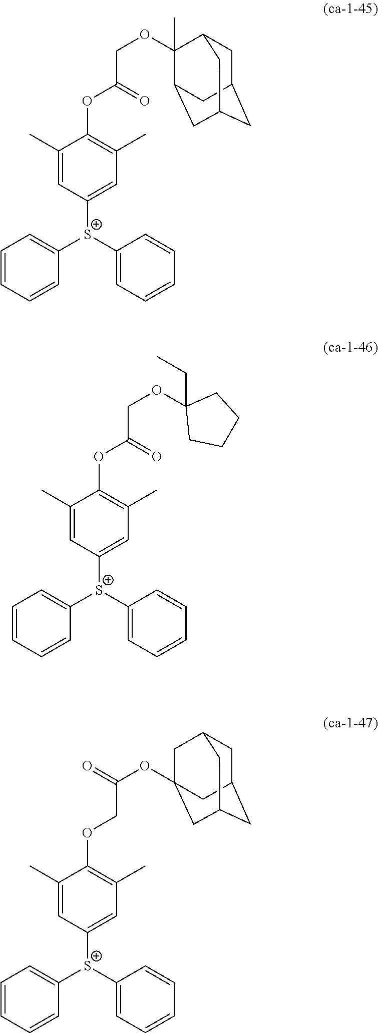 [SMILES: Cc1cc([S+](c2ccccc2)c2ccccc2)cc(C)c1OC(=O)COC1(C)C2CC3CC(C2)CC1C3 ; CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[In the formulae, g1, g2, and g3 represent a repeating number, g1 represents an integer of 1 to 5, g2 represents an integer of 0 to 20, and g3 represents an integer of 0 to 20.]

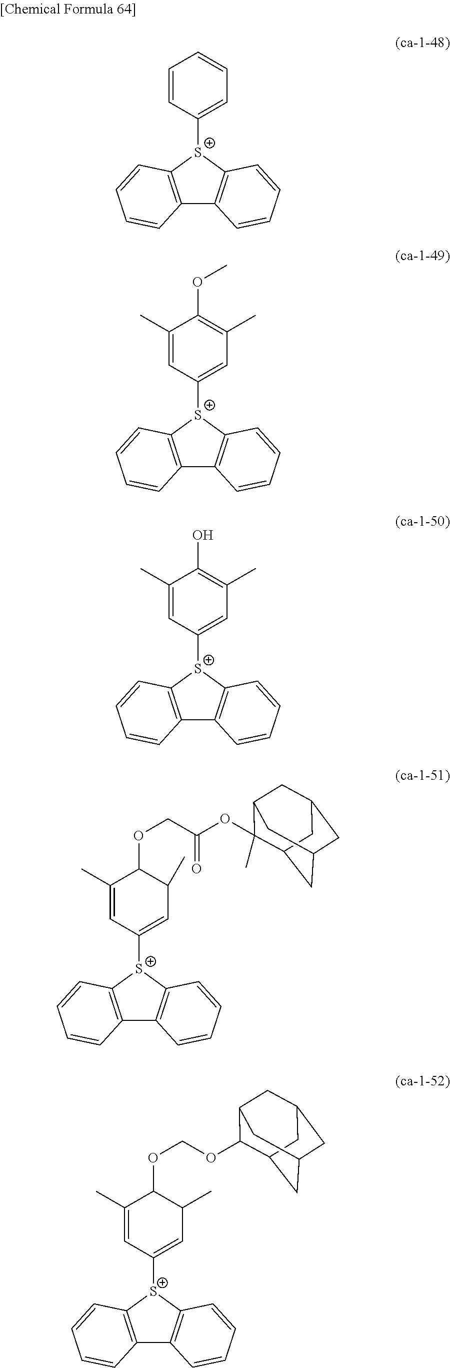 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CC1=CC([s+]2c3ccccc3c3ccccc32)=CC(C)C1OCOC1C2CC3CC(C2)CC1C3]

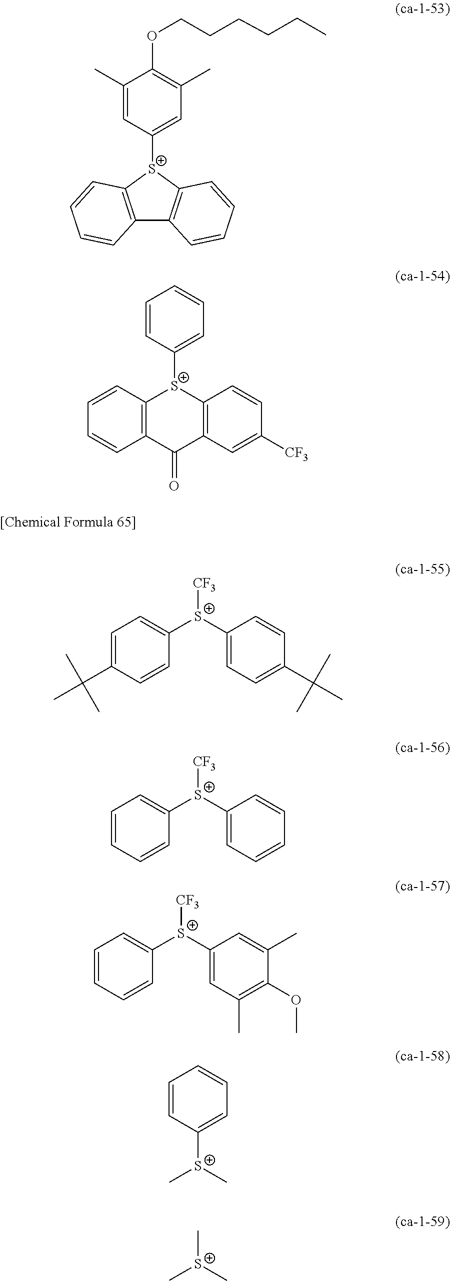

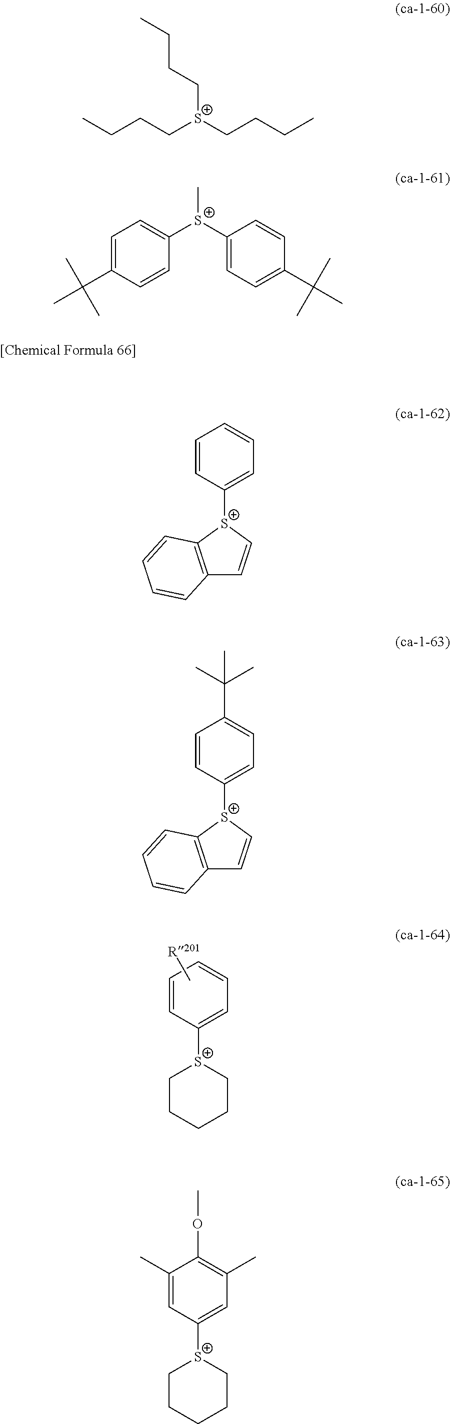

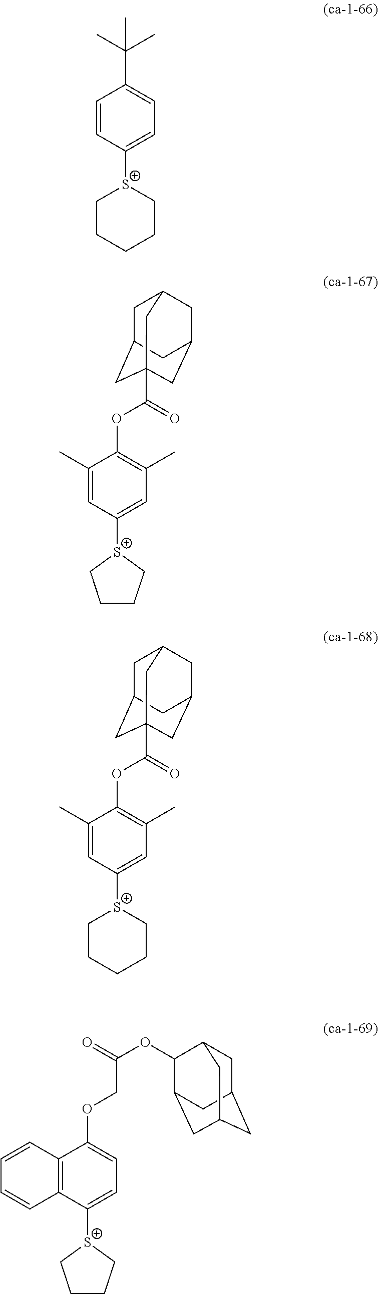 [SMILES: CC(C)(C)c1ccc([S+]2CCCCC2)cc1 ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

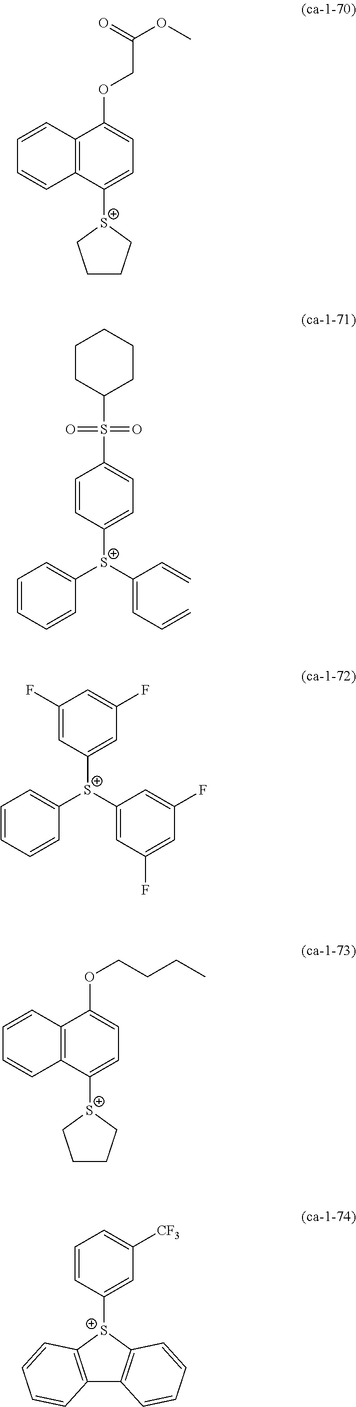 [SMILES: COC(=O)COc1ccc([S+]2CCCC2)c2ccccc12 ; C=C/C=C(\C=C)[S+](c1ccccc1)c1ccc(S(=O)(=O)C2CCCCC2)cc1 ; Fc1cc(F)cc([S+](c2ccccc2)c2cc(F)cc(F)c2)c1 ; CCCCOc1ccc([S+]2CCCC2)c2ccccc12 ; FC(F)(F)c1cccc(-[s+]2c3ccccc3c3ccccc32)c1]

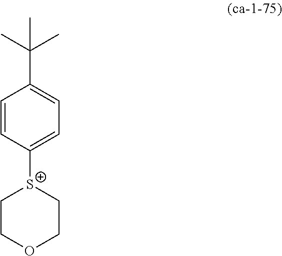 [SMILES: CC(C)(C)c1ccc([S+]2CCOCC2)cc1]

[In the formulae, R″²⁰¹ represents a hydrogen atom or a substituent, and examples of the substituent include the same groups as those for the substituents which may be included in R²⁰¹ to R²⁰⁷ and R²¹⁰ to R²¹².]

Specific examples of suitable cations represented by Formula (ca-2) include a diphenyliodonium cation and a bis(4-tert-butylphenyl) iodonium cation.

Specific examples of suitable cations represented by Formula (ca-3) include cations each represented by Formulae (ca-3-1) to (ca-3-6).

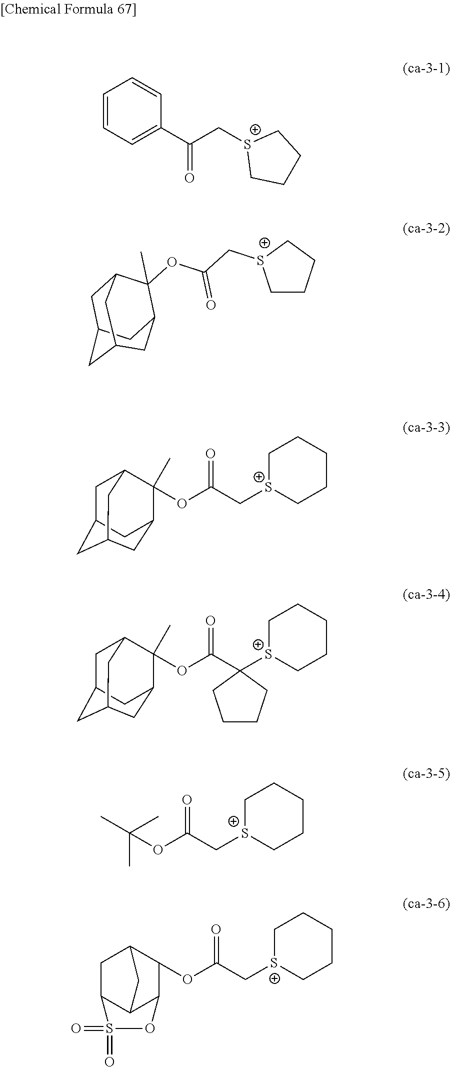 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

Among the examples, as the cation moiety ((M^{m+})_{l/m}), a cation represented by General Formula (ca-1) is preferable.

Specific examples of the component (B0) are shown below but are not limited thereto.

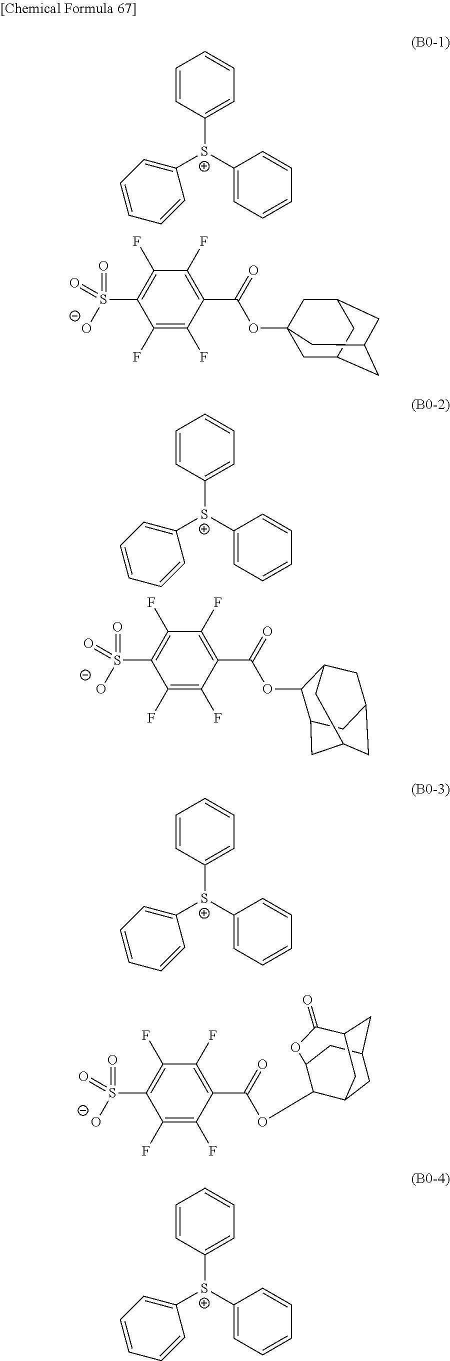

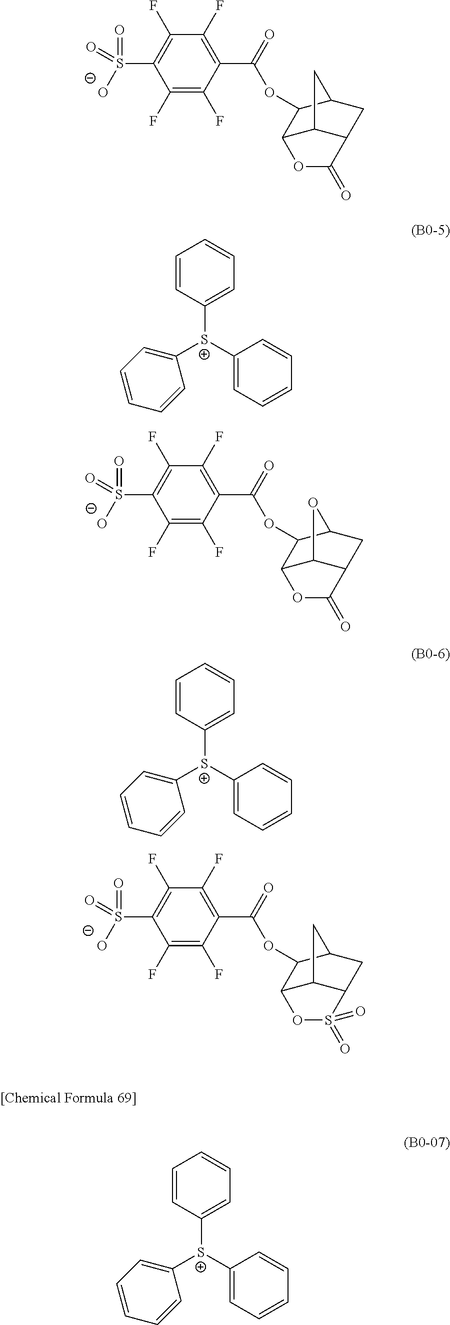 [SMILES: O=C(OC1C2CC3C(=O)OC1C3C2)c1c(F)c(F)c(S(=O)(=O)[O-])c(F)c1F ; c1ccc([S+](c2ccccc2)c2ccccc2)cc1 ; O=C(OC1C2CC3C(=O)OC1C3O2)c1c(F)c(F)c(S(=O)(=O)[O-])c(F)c1F ; c1ccc([S+](c2ccccc2)c2ccccc2)cc1 ; O=C(OC1C2CC3C1OS(=O)(=O)C3C2)c1c(F)c(F)c(S(=O)(=O)[O-])c(F)c1F ; c1ccc([S+](c2ccccc2)c2ccccc2)cc1]

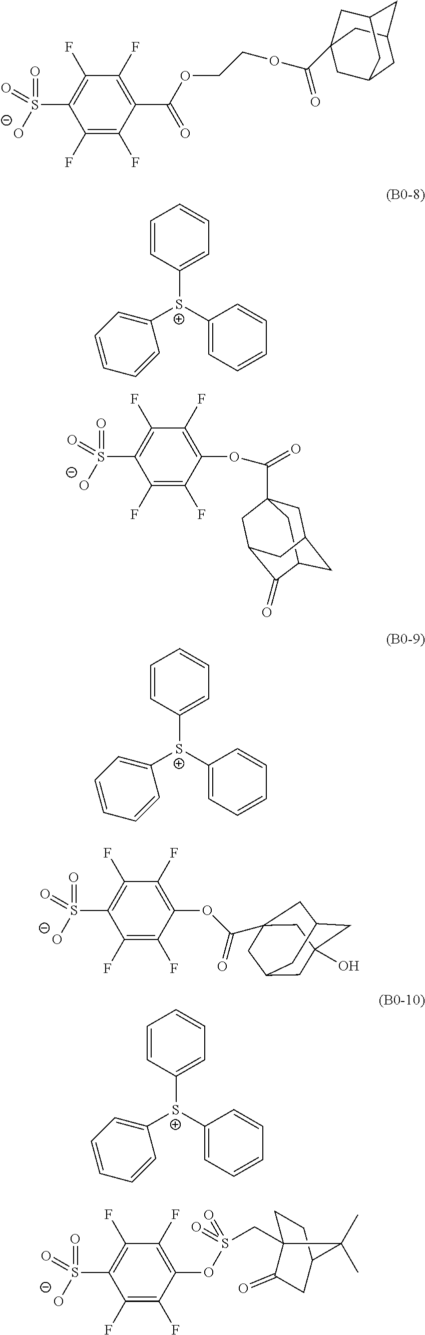

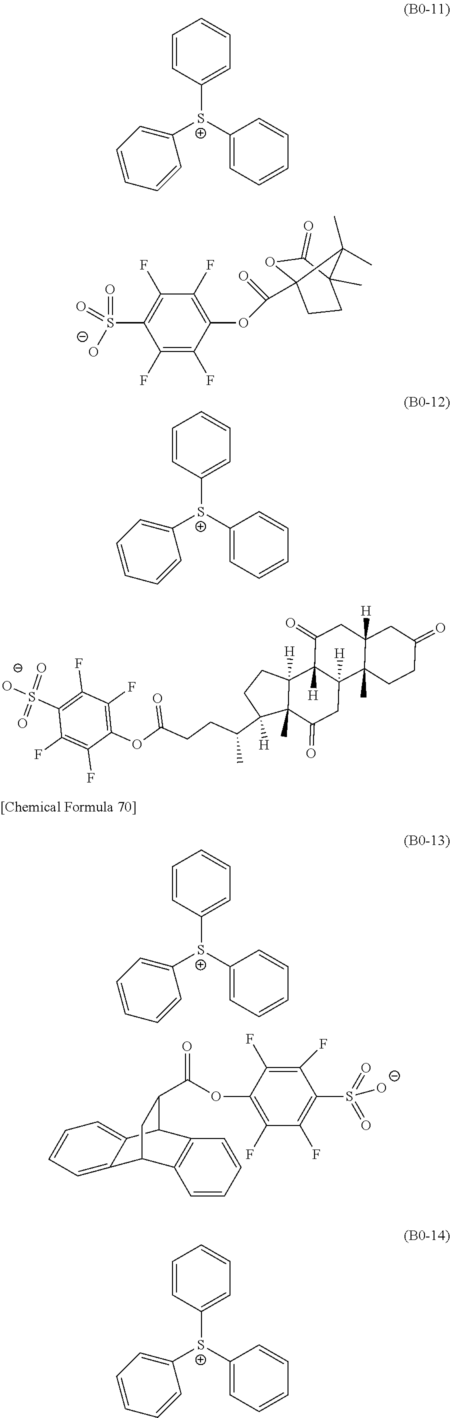

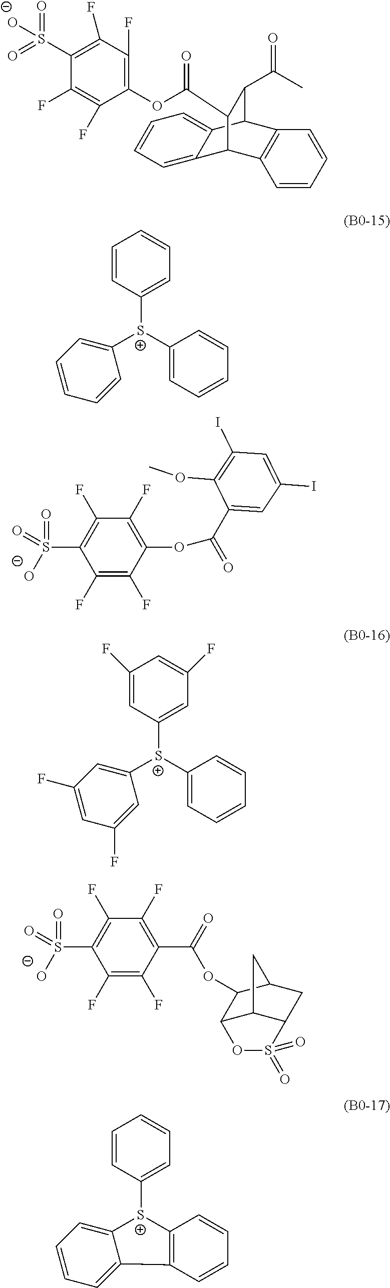 [SMILES: CC(=O)C1C2c3ccccc3C(c3ccccc32)C1C(=O)Oc1c(F)c(F)c(S(=O)(=O)[O-])c(F)c1F ; c1ccc([S+](c2ccccc2)c2ccccc2)cc1 ; COc1c(I)cc(I)cc1C(=O)Oc1c(F)c(F)c(S(=O)(=O)[O-])c(F)c1F ; Fc1cc(F)cc([S+](c2ccccc2)c2cc(F)cc(F)c2)c1 ; O=C(OC1C2CC3C1OS(=O)(=O)C3C2)c1c(F)c(F)c(S(=O)(=O)[O-])c(F)c1F ; c1ccc(-[s+]2c3ccccc3c3ccccc32)cc1]

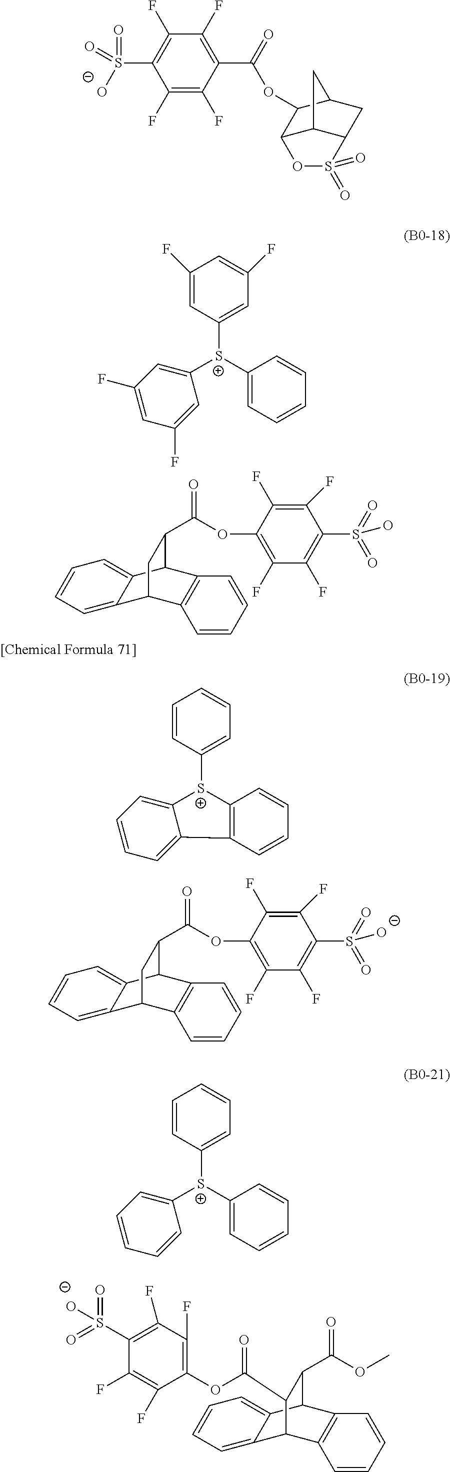

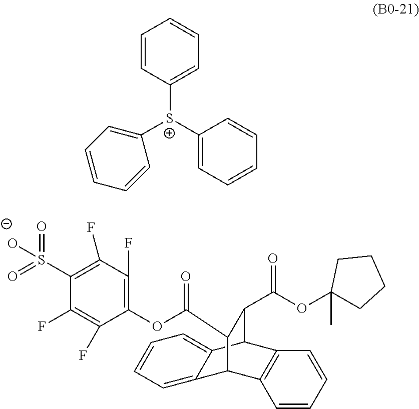 [SMILES: c1ccc([S+](c2ccccc2)c2ccccc2)cc1 ; CC1(OC(=O)C2C3c4ccccc4C(c4ccccc43)C2C(=O)Oc2c(F)c(F)c(S(=O)(=O)[O-])c(F)c2F)CCCC1]

In the resist composition according to the present embodiment, the component (B0) may be used alone or in a combination of two or more kinds thereof.

The content of the component (B0) in the resist composition according to the present embodiment is preferably in a range of 5 to 40 parts by mass, more preferably in a range of 10 to 40 parts by mass, still more preferably in a range of 15 to 40 parts by mass, and particularly preferably in a range of 20 to 35 parts by mass with respect to 100 parts by mass of the component (A).

In a case where the content of the component (B0) is greater than or equal to the lower limits of the above-described preferable ranges, the sensitivity and the lithography characteristics such as a line width roughness (LWR) reducing property are further improved in the resist pattern formation. Meanwhile, in a case where the content thereof is less than or equal to the upper limits of the above-described preferable ranges, a uniform solution is easily obtained, and the storage stability of the resist composition is further improved in a case of dissolving each component of the resist composition in an organic solvent.

The proportion of the component (B0) in the entire component (B) is, for example, 50% by mass or greater, preferably 70% by mass or greater, and more preferably 95% by mass or greater. The proportion of the component (B0) in the entire component (B) may be 100% by mass.

The component (B) may contain an acid generator component (B1) (hereinafter, also referred to as “component B1)”) other than the above-described component (B0).

Examples of the component (B1) are numerous and include onium salt-based acid generators such as iodonium salts and sulfonium salts; oxime sulfonate-based acid generators; diazomethane-based acid generators such as bisalkyl or bisaryl sulfonyl diazomethanes and poly(bis-sulfonyl)diazomethanes; nitrobenzyl sulfonate-based acid generators; iminosulfonate-based acid generators; and disulfone-based acid generators.

Examples of the onium salt-based acid generator as the component (B1) include a compound represented by any of General Formulae (b-1) to (b-3).

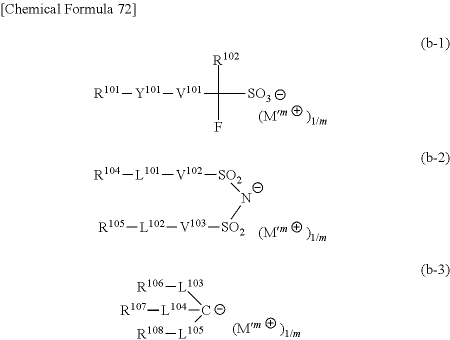

[In the formulae, R¹⁰¹ and R¹⁰⁴ to R¹⁰⁸ each independently represent a cyclic group which may have a substituent, a chain-like alkyl group which may have a substituent, or a chain-like alkenyl group which may have a substituent. R¹⁰⁴ and R¹⁰⁵ may be bonded to each other to form a ring structure. R¹⁰² represents a fluorinated alkyl group having 1 to 5 carbon atoms or a fluorine atom. Y¹⁰¹ represents a divalent linking group having an oxygen atom or a single bond. V¹⁰¹ to V¹⁰³ each independently represent a single bond, an alkylene group, or a fluorinated alkylene group. Here, Y¹⁰¹ and V¹⁰¹ do not represent a single bond at the same time. L¹⁰¹ and L¹⁰² each independently represent a single bond or an oxygen atom. L¹⁰³ to L¹⁰⁵ each independently represent a single bond, —CO—, or —SO₂—. m represents an integer of 1 or greater, and M′^{m+} represents an m-valent onium cation.]

In the resist composition according to the present embodiment, the component (B1) may be used alone or a combination of two or more kinds thereof may be used.

In a case where the resist composition of the present embodiment contains the component (B1), the proportion of the component (B1) in the entire component (B) is preferably less than 50% by mass, more preferably 40% by mass or less, still more preferably 30% by mass or less, and particularly preferably less than 20% by mass.

From the viewpoint of environmental burden, it is preferable that the resist composition of the present embodiment does not contain the component (B1).

### <Other Components>

The resist composition of the present embodiment may further contain other components in addition to the component (A) and the component (B) described above. Examples of other components include a component (D), a component (E), a component (F), and a component(S) described below.

### <<Base Component (D)>>

The resist composition according to the present embodiment may further contain, in addition to the components (A) and (B), a base component (component (D)) that traps (that is, controls the diffusion of an acid) an acid generated upon light exposure. The component (D) acts as a quencher (an acid diffusion control agent) which traps the acid generated in the resist composition upon light exposure.

Examples of the component (D) include a photodecomposable base (D1) having acid diffusion controllability (hereinafter, referred to as “component (D1)”) which is lost by the decomposition upon light exposure and a nitrogen-containing organic compound (D2) (hereinafter, referred to as “component (D2)”) which does not correspond to the component (D1). Among these, the photodecomposable base (component (D1)) is preferable from the viewpoint of easily increasing the sensitivity, reducing the roughness, and improving the characteristic of suppressing occurrence of coating defects.

### In Regard to Component (D1)

The component (D1) is not particularly limited as long as the component is decomposed upon light exposure and loses an acid diffusion controllability, and one or more compounds selected from the group consisting of a compound represented by General Formula (d1-1) (hereinafter, referred to as “component (d1-1)”), a compound represented by General Formula (d1-2) (hereinafter, referred to as “component (d1-2)”), and a compound represented by General Formula (d1-3) (hereinafter, referred to as “component (d1-3)”) are preferable.

Since the components (d1-1) to (d1-3) are decomposed and lose the acid diffusion controllability (basicity), the components (d1-1) to (d1-3) do not act as a quencher at the exposed portion of the resist film, but act as a quencher at the unexposed portion of the resist film.

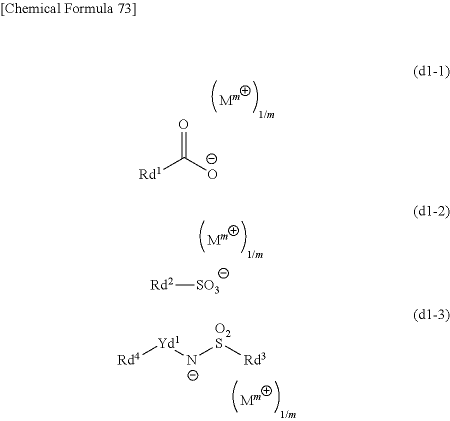

[In the formulae, Rd¹ to Rd⁴ represent a cyclic group which may have a substituent, a chain-like alkyl group which may have a substituent, or a chain-like alkenyl group which may have a substituent. Here, the carbon atom adjacent to the S atom as Rd² in Formula (d1-2) has no fluorine atom bonded thereto. Yd¹ represents a single bond or a divalent linking group. m represents an integer of 1 or greater, and each M^{m+} independently represents an m-valent organic cation.]

{Component (d1-1)}

### Anion Moiety

In Formula (d1-1), Rd¹ represents a cyclic group which may have a substituent, a chain-like alkyl group which may have a substituent, or a chain-like alkenyl group which may have a substituent, and examples thereof include the same groups as those for R′²⁰¹.

Among these, it is preferable that the group as Rd¹ represents an aromatic hydrocarbon group which may have a substituent, an aliphatic cyclic group which may have a substituent, or a chain-like alkyl group which may have a substituent. Examples of the substituent that may be included in these groups include a hydroxyl group, an oxo group, an alkyl group, an aryl group, a fluorine atom, a fluorinated alkyl group, a lactone-containing cyclic group represented by any of General Formulae (a2-r-1) to (a2-r-7), an ether bond, an ester bond, and a combination thereof. In a case where an ether bond or an ester bond is included as the substituent, the substituent may be bonded through an alkylene group, and a linking group represented by any of Formulae (y-a1-1) to (y-a1-5) is preferable as the substituent. Further, in a case where the aromatic hydrocarbon group, the aliphatic cyclic group, or the chain-like alkyl group as Rd¹ contains a linking group represented by any of General Formulae (y-a1-1) to (y-a1-7) as a substituent, V′¹⁰¹ in General Formula (y-a1-1) to (y-a1-7) is bonded to the carbon atom constituting the aromatic hydrocarbon group, the aliphatic cyclic group, or the chain-like alkyl group as Rd¹ in General Formula (d3-1), in Formulae (y-a1-1) to (y-a1-7).

Suitable examples of the aromatic hydrocarbon group include a phenyl group, a naphthyl group, and a polycyclic structure having a bicyclooctane skeleton (for example, a polycyclic structure formed of a bicyclooctane skeleton and a ring structure other than the bicyclooctane skeleton).

As the aliphatic cyclic group, a group in which one or more hydrogen atoms have been removed from a polycycloalkane such as adamantane, norbornane, isobornane, tricyclo[5.2.1.0^{2,6}]decane, or tetracyclododecane is more preferable.

It is preferable that the chain-like alkyl group has 1 to 10 carbon atoms, and specific examples thereof include a linear alkyl group such as a methyl group, an ethyl group, a propyl group, a butyl group, a pentyl group, a hexyl group, a heptyl group, an octyl group, a nonyl group, or a decyl group; and a branched alkyl group such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, or a 4-methylpentyl group.

In a case where the chain-like alkyl group is a fluorinated alkyl group having a fluorine atom or a fluorinated alkyl group as a substituent, the fluorinated alkyl group has preferably 1 to 11 carbon atoms, more preferably 1 to 8 carbon atoms, and still more preferably 1 to 4 carbon atoms. The fluorinated alkyl group may have an atom other than a fluorine atom. Examples of the atom other than a fluorine atom include an oxygen atom, a sulfur atom, and a nitrogen atom.

Specific preferred examples of the anion moiety in the component (d1-1) are described below.

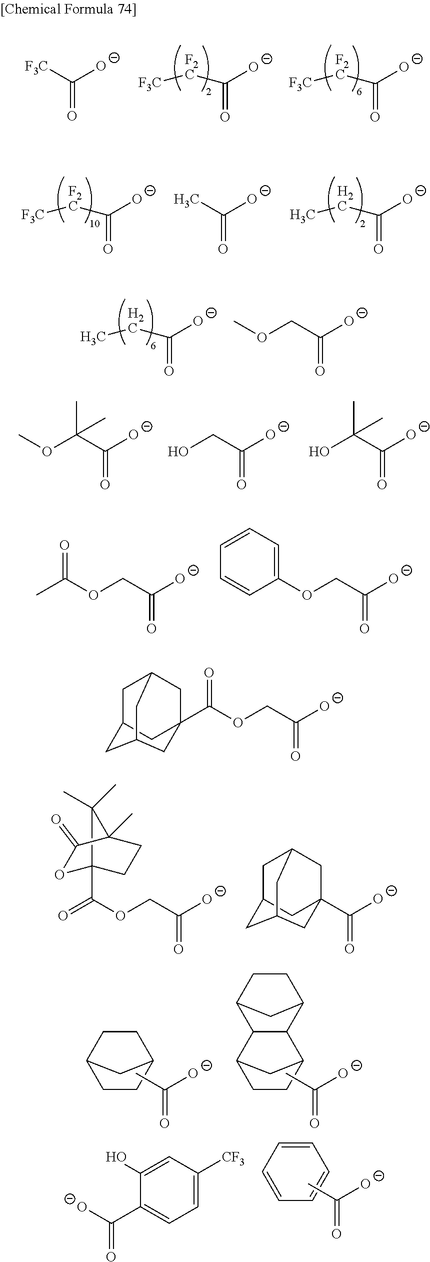

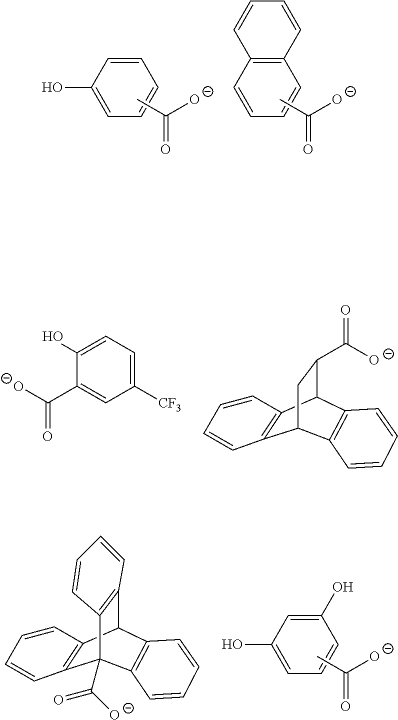

### Cation Moiety

In Formula (d1-1), M^{m+} represents an m-valent organic cation.

Examples of the organic cation as M^{m+} include the same cations as those for the cations each represented by General Formulae (ca-1) to (ca-3). Among these, a cation represented by General Formula (ca-1) is more preferable, and cations each represented by Formulae (ca-1-1) to (ca-1-75) are still more preferable.

The component (d1-1) may be used alone or a combination of two or more kinds thereof may be used.

{Component (d1-2)}

### Anion Moiety

In Formula (d1-2), Rd² represents a cyclic group which may have a substituent, a chain-like alkyl group which may have a substituent, or a chain-like alkenyl group which may have a substituent, and examples thereof include the same groups as those for R′²⁰¹.

Here, the carbon atom adjacent to the S atom in Rd² has no fluorine atom bonded thereto (the carbon atom is not substituted with a fluorine atom). As a result, the anion of the component (d1-2) becomes an appropriately weak acid anion, thereby improving the quenching ability of the component (D).

Rd² represents preferably a chain-like alkyl group which may have a substituent or an aliphatic cyclic group which may have a substituent and more preferably an aliphatic cyclic group which may have a substituent.

The chain-like alkyl group preferably has 1 to 10 carbon atoms and more preferably 3 to 10 carbon atoms.

As the aliphatic cyclic group, a group (which may have a substituent) in which one or more hydrogen atoms have been removed from adamantane, norbornane, isobornane, tricyclo[5.2.1.0^{2,6}]decane, or tetracyclododecane; and a group in which one or more hydrogen atoms have been removed from camphor is more preferable.

The hydrocarbon group as Rd² may have a substituent, and examples of the substituent include the same substituents as those that the hydrocarbon group (such as an aromatic hydrocarbon group, an aliphatic cyclic group, or a chain-like alkyl group) as Rd¹ in Formula (d1-1) may have.

Specific preferred examples of the anion moiety in the component (d1-2) are described below.

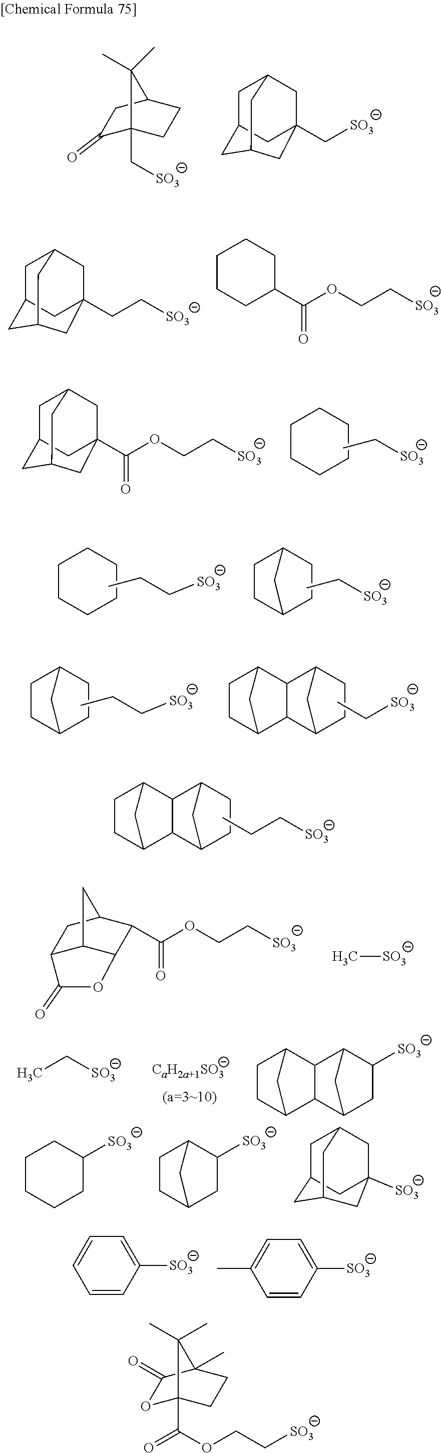

### Cation Moiety

In Formula (d1-2), M^{m+} represents an m-valent organic cation and has the same definition as that for M^{m+} in Formula (d1-1).

The component (d1-2) may be used alone or a combination of two or more kinds thereof may be used.

{Component (d1-3)}

### Anion Moiety

In Formula (d1-3), Rd³ represents a cyclic group which may have a substituent, a chain-like alkyl group which may have a substituent, or a chain-like alkenyl group which may have a substituent, and examples thereof include the same groups as those for R′²⁰¹. Among these, a cyclic group having a fluorine atom, a chain-like alkyl group, or a chain-like alkenyl group is preferable. Among these, a fluorinated alkyl group is preferable, and the same groups as those for the fluorinated alkyl group represented by Rd¹ are more preferable.

In Formula (d1-3), Rd⁴ represents a cyclic group which may have a substituent, a chain-like alkyl group which may have a substituent, or a chain-like alkenyl group which may have a substituent, and examples thereof include the same groups as those for R′²⁰¹.

Among these, an alkyl group which may have a substituent, an alkoxy group which may have a substituent, an alkenyl group which may have a substituent, or a cyclic group which may have a substituent is preferable.

It is preferable that the alkyl group as Rd⁴ is a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group as Rd⁴ may be substituted with a hydroxyl group, a cyano group, or the like.

It is preferable that the alkoxy group as Rd⁴ is an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among these, a methoxy group and an ethoxy group are preferable.

Examples of the alkenyl group as Rd⁴ include the same groups as those for the alkenyl group as R′²⁰¹. Among these, a vinyl group, a propenyl group (an allyl group), a 1-methylpropenyl group, and a 2-methylpropenyl group are preferable. These groups may have an alkyl group having 1 to 5 carbon atoms or a halogenated alkyl group having 1 to 5 carbon atoms as a substituent.

Examples of the cyclic group as Rd⁴ include the same groups as those for the cyclic group as R′²⁰¹. Among these, an alicyclic group in which one or more hydrogen atoms have been removed from a cycloalkane such as cyclopentane, cyclohexane, adamantane, norbornane, isobornane, tricyclo[5.2.1.0^{2,6}]decane, or tetracyclododecane or an aromatic group such as a phenyl group or a naphthyl group is preferable. In a case where Rd⁴ represents an alicyclic group, the resist composition is satisfactorily dissolved in an organic solvent so that the lithography characteristics are improved. Further, in a case where Rd⁴ represents an aromatic group, the resist composition has excellent light absorption efficiency in lithography using EUV or the like as an exposure light source, and thus the sensitivity and lithography characteristics are improved.

In Formula (d1-3), Yd¹ represents a single bond or a divalent linking group.

The divalent linking group as Yd¹ is not particularly limited, and examples thereof include a divalent hydrocarbon group (an aliphatic hydrocarbon group or an aromatic hydrocarbon group) which may have a substituent and a divalent linking group having a heteroatom. These divalent linking groups are the same as those for the divalent hydrocarbon group which may have a substituent and the divalent linking group having a heteroatom described in the section of the divalent linking group as Ya²¹ in Formula (a2-1).

It is preferable that Yd¹ represents a carbonyl group, an ester bond, an amide bond, an alkylene group, or a combination of these. As the alkylene group, a linear or branched alkylene group is more preferable, and a methylene group or an ethylene group is still more preferable.

Specific preferred examples of the anion moiety in the component (d1-3) are described below.

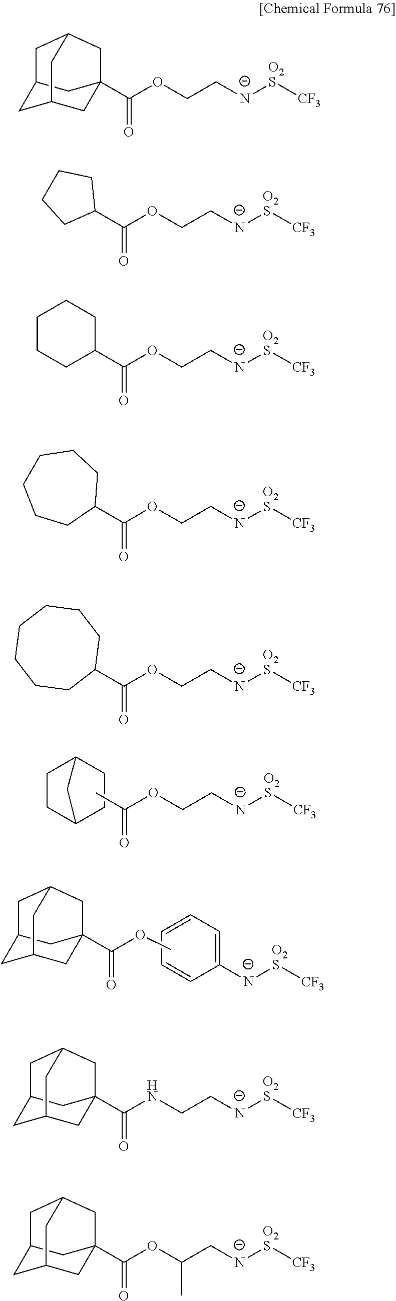

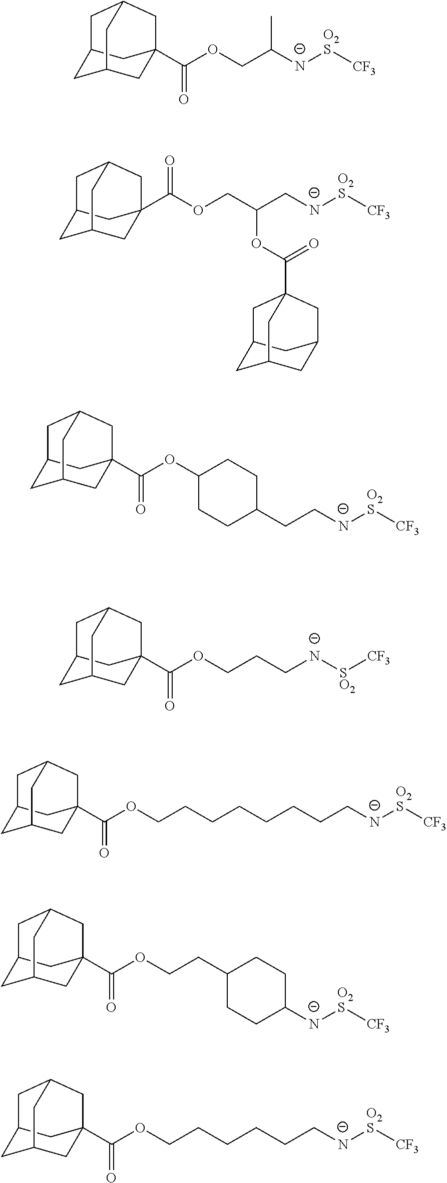

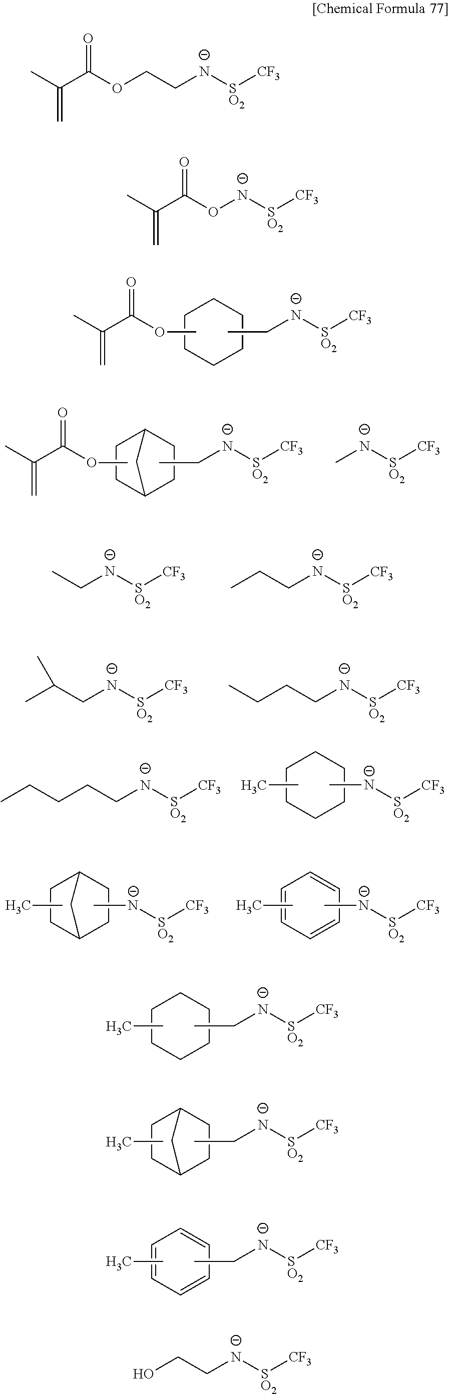

### Cation Moiety

In Formula (d1-3), M^{m+} represents an m-valent organic cation and has the same definition as that for M^{m+} in Formula (d1-1).

The component (d1-3) may be used alone or a combination of two or more kinds thereof may be used.

As the component (D1), only one of the above-described components (d1-1) to (d1-3) or a combination of two or more kinds thereof may be used.

In a case where the resist composition contains the component (D1), the content of the component (D1) in the resist composition is preferably in a range of 0.5 to 15 parts by mass, more preferably in a range of 1 to 12 parts by mass, and still more preferably in a range of 2 to 10 parts by mass with respect to 100 parts by mass of the component (A).

It is preferable that the component (D1) contains the component (d1-1).

The content of the component (d1-1) in the total component (D1) is preferably 50% by mass or more, preferably 70% by mass or more, and still more preferably 90% by mass or more, and the component (D1) may consist of only a compound for the component (d1-1).

### Method of Producing Component (D1):

The methods of producing the component (d1-1) and the component (d1-2) are not particularly limited, and these components can be produced by known methods.

Further, the method of producing the component (d1-3) is not particularly limited, and the component is produced by the same method as disclosed in United States Patent Application, Publication No. 2012-0149916.

### In Regard to Component (D2)

The component (D) may contain a nitrogen-containing organic compound component (hereinafter, referred to as a “component (D2)”) which does not correspond to the component (D1).

The component (D2) is not particularly limited as long as it acts as an acid diffusion control agent and does not correspond to the component (D1), and any known compound may be used. Among the examples, an aliphatic amine is preferable, and particularly a secondary aliphatic amine and a tertiary aliphatic amine are more preferable.

The aliphatic amine is an amine containing one or more aliphatic groups, and the number of carbon atoms in the aliphatic group is preferably in a range of 1 to 12.

Examples of these aliphatic amines include amines in which at least one hydrogen atom of ammonia NH₃ has been substituted with an alkyl group or hydroxyalkyl group having 12 or less carbon atoms (alkylamines or alkylalcoholamines), and cyclic amines.

Specific examples of the alkylamines and the alkylalcoholamines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkylalcoholamines such as diethanolamine, triethanolamine, diisopropanolamine, triisopropanolamine, di-n-octanolamine, and tri-n-octanolamine. Among these, a trialkylamine having 6 to 30 carbon atoms is still more preferable, and tri-n-pentylamine or tri-n-octylamine is particularly preferable.

Examples of the cyclic amine include a heterocyclic compound having a nitrogen atom as a heteroatom. The heterocyclic compound may be a monocyclic compound (aliphatic monocyclic amine) or a polycyclic compound (aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris {2-(2-methoxyethoxy)ethyl}amine, tris {2-(2-methoxyethoxymethoxy)ethyl}amine, tris {2-(1-methoxyethoxy)ethyl}amine, tris {2-(1-ethoxyethoxy)ethyl}amine, tris {2-(1-ethoxypropoxy)ethyl}amine, tris[2-{2-(2-hydroxyethoxy) ethoxy}ethyl]amine, and triethanolamine triacetate, and triethanolamine triacetate and 2,6-di-tert-butylpyridine are preferable.

As the component (D2), an aromatic amine may be used.

Examples of the aromatic amine include 4-dimethylaminopyridine, pyrrole, indole, pyrazole, imidazole, and derivatives thereof, tribenzylamine, 2,6-diisopropylaniline, N-tert-butoxycarbonylpyrrolidine, 2,6-di-tert-butylpyridine, and 2,6-tert-butylpyridine.

The component (D2) may be used alone or a combination of two or more kinds thereof may be used.

In a case where the resist composition contains the component (D2), the content of the component (D2) in the resist composition is typically in a range of 0.01 to 5 parts by mass with respect to 100 parts by mass of the component (A1). In a case where the content thereof is set to be in the above-described range, the resist pattern shape, the post-exposure temporal stability, and the like are improved.

<<At Least One Compound (E) Selected from Group Consisting of Organic Carboxylic Acids, Phosphorus Oxo Acids, and Derivatives Thereof>>

For the purpose of preventing any deterioration in sensitivity and improving the resist pattern shape and the post-exposure temporal stability, the resist composition according to the present embodiment may contain, as an optional component, at least one compound (E) (hereinafter referred to as “component (E)”) selected from the group consisting of an organic carboxylic acid, and a phosphorus oxo acid and a derivative thereof.

Specific examples of the organic carboxylic acid include acetic acid, malonic acid, citric acid, malic acid, succinic acid, benzoic acid, and salicylic acid. Among these, salicylic acid is preferable.

Examples of the phosphorus oxo acid include phosphoric acid, phosphonic acid, and phosphinic acid. Among these, phosphonic acid is particularly preferable.

In the resist composition of the present embodiment, the component (E) may be used alone or a combination of two or more kinds thereof may be used.

In a case where the resist composition contains the component (E), the content of the component (E) is preferably in a range of 0.01 to 5 parts by mass and more preferably in a range of 0.05 to 3 parts by mass with respect to 100 parts by mass of the component (A). In a case where the content thereof is in the above-described ranges, the lithography characteristics are further improved.

### <<Fluorine Additive Component (F)>>

The resist composition according to the present embodiment may further contain a fluorine additive component (hereinafter, referred to as “component (F)”) as a hydrophobic resin. The component (F) is used to impart water repellency to the resist film and used as a resin different from the component (A), whereby the lithography characteristics can be improved.

As the component (F), for example, the fluorine-containing polymer compounds described in Japanese Unexamined Patent Application, First Publication Nos. 2010-002870, 2010-032994, 2010-277043, 2011-13569, and 2011-128226 can be used.

Specific examples of the component (F) include a polymer having a constitutional unit (f1) represented by General Formula (f1-1). As the polymer, a polymer (homopolymer) formed of only the constitutional unit (f1) represented by Formula (f1-1); a copolymer of the constitutional unit (f1) and the constitutional unit (a1); or a copolymer of the constitutional unit (f1), a constitutional unit derived from acrylic acid or methacrylic acid, and the constitutional unit (a1) is preferable, and a copolymer of the constitutional unit (f1) and the constitutional unit (a1) is more preferable. Here, as the constitutional unit (a1) copolymerized with the constitutional unit (f1), a constitutional unit derived from 1-ethyl-1-cyclooctyl (meth)acrylate or a constitutional unit derived from 1-methyl-1-adamantyl (meth)acrylate is preferable, and a constitutional unit derived from 1-ethyl-1-cyclooctyl (meth)acrylate is more preferable.

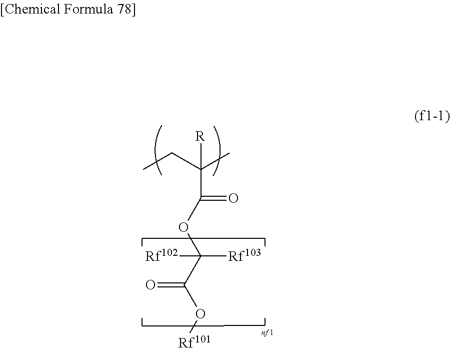

[In the formula, R has the same definition as described above, Rf¹⁰² and Rf¹⁰³ each independently represent a hydrogen atom, a halogen atom, an alkyl group having 1 to 5 carbon atoms, or a halogenated alkyl group having 1 to 5 carbon atoms, and Rf¹⁰² and Rf¹⁰³ may be the same as or different from each other. nf¹ represents an integer of 0 to 5, and Rf¹⁰¹ represents an organic group having a fluorine atom.]

In Formula (f1-1), R bonded to the carbon atom at the α-position has the same definition as described above. It is preferable that R represents a hydrogen atom or a methyl group.

In Formula (f1-1), a fluorine atom is preferable as the halogen atom as Rf¹⁰² and Rf¹⁰³. Examples of the alkyl group having 1 to 5 carbon atoms as Rf¹⁰² and Rf¹⁰³ include the same groups as those for the alkyl group having 1 to 5 carbon atoms as R. Among the examples, a methyl group or an ethyl group is preferable. Specific examples of the halogenated alkyl group having 1 to 5 carbon atoms for Rf¹⁰² and Rf¹⁰³ include groups in which some or all hydrogen atoms of an alkyl group having 1 to 5 carbon atoms have been substituted with halogen atoms. Among these, a fluorine atom is preferable as the halogen atom. Among these, Rf¹⁰² and Rf¹⁰³ represent preferably a hydrogen atom, a fluorine atom, or an alkyl group having 1 to 5 carbon atoms, more preferably a hydrogen atom, a fluorine atom, a methyl group, or an ethyl group, and still more preferably a hydrogen atom.

In Formula (f1-1), nf¹ represents an integer of 0 to 5, preferably an integer of 0 to 3, and more preferably 1 or 2.

In Formula (f1-1), Rf¹⁰¹ represents an organic group having a fluorine atom and preferably a hydrocarbon group having a fluorine atom.

The hydrocarbon group having a fluorine atom may be linear, branched, or cyclic, and the number of carbon atoms thereof is preferably in a range of 1 to 20, more preferably in a range of 1 to 15, and particularly preferably in a range of 1 to 10.

In the hydrocarbon group having a fluorine atom, preferably 25% or greater of the hydrogen atoms in the hydrocarbon group are fluorinated, more preferably 50% or greater thereof are fluorinated, and still more preferably 60% or greater thereof are fluorinated from the viewpoint of increasing the hydrophobicity of the resist film during immersion exposure.

Among examples, Rf¹⁰¹ represents more preferably a fluorinated hydrocarbon group having 1 to 6 carbon atoms and particularly preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃.

The weight-average molecular weight (Mw) (in terms of polystyrene according to gel permeation chromatography) of the component (F) is preferably in a range of 1000 to 50000, more preferably in a range of 5000 to 40000, and most preferably in a range of 10000 to 30000. In a case where the weight-average molecular weight thereof is set to be less than or equal to the upper limits of the above-described ranges, the resist composition exhibits a satisfactory solubility in a solvent for a resist enough to be used as a resist. Meanwhile, in a case where the weight-average molecular weight thereof is set to be greater than or equal to the lower limits of the above-described ranges, water repellency of the resist film is satisfactory.

Further, the dispersity (Mw/Mn) of the component (F) is preferably in a range of 1.0 to 5.0, more preferably in a range of 1.0 to 3.0, and most preferably in a range of 1.0 to 2.5.

In the resist composition according to the present embodiment, the component (F) may be used alone or a combination of two or more kinds thereof may be used.

In a case where the resist composition contains the component (F), the content of the component (F) is preferably in a range of 0.5 to 10 parts by mass and more preferably in a range of 1 to 10 parts by mass with respect to 100 parts by mass of the component (A).

### <<Organic Solvent Component (S)>>

The resist composition according to the present embodiment may be produced by dissolving the resist materials in an organic solvent component (hereinafter, referred to as a “component(S)”).

In the resist composition of the present embodiment, the component(S) may be used alone or in the form of a mixed solvent of two or more kinds thereof. Among these, PGMEA, PGME, γ-butyrolactone, EL, or cyclohexanone is preferable.

Further, a mixed solvent obtained by mixing PGMEA with a polar solvent is also preferable as the component(S). The blending ratio (mass ratio) may be appropriately determined in consideration of the compatibility between PGMEA and the polar solvent.

A mixed solvent of at least one selected from PGMEA and EL, and γ-butyrolactone is also preferable as the(S) component. In this case, as the mixing ratio, the mass ratio between the former and the latter is preferably in a range of 70:30 to 95:5.

The amount of the component(S) to be used is not particularly limited and is appropriately set to have a concentration which enables coating a substrate or the like depending on the thickness of the coated film. The component(S) is typically used in an amount such that the solid content concentration of the resist composition is set to be in a range of 0.1% to 20% by mass and preferably in a range of 0.2% to 15% by mass.

After the resist material is dissolved in the component(S), impurities may be removed from the resist composition of the present embodiment using a porous polyimide film, a porous polyamideimide film, or the like. For example, the resist composition may be filtered using a filter formed of a porous polyimide film, a filter formed of a porous polyamideimide film, a filter formed of a porous polyimide film and a porous polyamideimide film, or the like. Examples of the porous polyimide film and the porous polyamideimide film include those described in Japanese Unexamined Patent Application, First Publication No. 2016-155121.

The resist composition of the present embodiment described above contains the compound (B0) as the acid generator component, and thus, the lithography characteristics such as LWR can be improved while satisfactory sensitivity is maintained.

The compound (B0) has a bulky structure by having an aromatic ring containing a fluorine atom as a substituent between the cyclic group and the sulfonic acid group. It is assumed that the acid strength can be sufficiently obtained and the lithography characteristics can be improved by such a bulky structure.

In addition, it is considered that since the carbon atom adjacent to the sulfonic acid group is not fluorinated in the compound (B0), the risk of future use restriction and production restriction is low.

### (Method for Forming Resist Pattern)

A resist pattern formation method according to a second aspect of the present invention is a method including a step of forming a resist film on a support using the resist composition according to the first aspect of the present invention described above, a step of exposing the resist film to light, and a step of developing the resist film exposed to light to form a resist pattern.

According to the embodiment of the resist pattern formation method, a resist pattern formation method which is performed in the following manner is an exemplary example.

First, a support is coated with the resist composition of the present embodiment using a spinner or the like, and a bake (post applied bake (PAB)) treatment is performed under a temperature condition of 80° C. to 150° C. for 40 to 120 seconds and preferably 60 to 90 seconds to form a resist film.

Next, the selective exposure is performed on the resist film by, for example, light exposure through a mask (mask pattern) having a predetermined pattern formed thereon using an exposure apparatus such as an electron beam lithography apparatus or an ArF exposure apparatus, or direct irradiation with an electron beam for drawing without using a mask pattern, and a bake treatment (post-exposure bake (PEB)) is carried out, for example, under a temperature condition of 80° C. to 150° C. for 40 to 120 seconds and preferably 60 to 90 seconds.

Next, the resist film is subjected to a developing treatment. The developing treatment is conducted using an alkali developing solution in a case of an alkali developing process and using a developing solution containing an organic solvent (organic developing solution) in a case of a solvent developing process.

After the developing treatment, it is preferable to conduct a rinse treatment. As the rinse treatment, water rinsing using pure water is preferable in a case of the alkali developing process, and rinsing using a rinse solution containing an organic solvent is preferable in a case of the solvent developing process.

In a case of the solvent developing process, after the developing treatment or the rinse treatment, the developing solution or the rinse solution attached onto the pattern may be removed by a treatment using a supercritical fluid.

After the developing treatment or the rinse treatment, drying is conducted. As desired, a bake treatment (post-bake) may be conducted after the developing treatment.

The support is not particularly limited and a known support of the related art can be used, and examples thereof include a substrate for an electronic component and a substrate on which a predetermined wiring pattern has been formed. Specific examples thereof include a metal substrate such as a silicon wafer, copper, chromium, iron, or aluminum; and a glass substrate. As the materials of the wiring pattern, copper, aluminum, nickel, or gold can be used.

The wavelength to be used for light exposure is not particularly limited and the exposure can be conducted using radiation such as an ArF excimer laser, a KrF excimer laser, an F2 excimer laser, extreme ultraviolet (EUV) rays, vacuum ultraviolet rays (VUV), electron beams (EB), X-rays, and soft X-rays.

The method of exposing the resist film to light may be general exposure (dry exposure) conducted in air or an inert gas such as nitrogen, or liquid immersion exposure (liquid immersion lithography).

The liquid immersion exposure is an exposure method in which the region between the resist film and the lens at the lowermost position of the exposure apparatus is filled with a solvent (liquid immersion medium) in advance that has a refractive index greater than the refractive index of air, and the exposure (immersion exposure) is conducted in this state.

As the liquid immersion medium, a solvent having a refractive index greater than the refractive index of air but less than the refractive index of the resist film to be exposed is preferable, and examples thereof include water, a fluorine-based inert liquid, a silicon-based solvent, and a hydrocarbon-based solvent.

As the liquid immersion medium, water is preferably used.

As the alkali developing solution used for the developing treatment in the alkali developing process, a 0.1 to 10 mass % tetramethylammonium hydroxide (TMAH) aqueous solution is an exemplary example.

The organic solvent contained in the organic developing solution used for the developing treatment in the solvent developing process may be any solvent that is capable of dissolving the component (A) (the component (A) before light exposure) and can be appropriately selected from known organic solvents. Specific examples thereof include a polar solvent such as a ketone-based solvent, an ester-based solvent, an alcohol-based solvent, a nitrile-based solvent, an amide-based solvent, and an ether-based solvent, and a hydrocarbon-based solvent.

Examples of the ester-based solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile-based solvent include acetonitrile, propionitrile, valeronitrile, and butyronitrile.

Known additives can be blended into the organic developing solution as necessary. Examples of the additive include a surfactant. The surfactant is not particularly limited, and for example, an ionic or non-ionic fluorine-based and/or silicon-based surfactant can be used.

The developing treatment can be performed according to a known developing method, and examples thereof include a method of immersing a support in a developing solution for a certain time (a dip method), a method of raising a developing solution on the surface of a support using the surface tension and maintaining the state for a certain time (a puddle method), a method of spraying a developing solution to the surface of a support (spray method), and a method of continuously ejecting a developing solution onto a support rotating at a certain rate while scanning a developing solution ejection nozzle at a certain rate (dynamic dispense method).

As the organic solvent contained in the rinse solution used for the rinse treatment after the developing treatment in the solvent developing process, a solvent that is unlikely to dissolve a resist pattern can be appropriately selected from the organic solvents described as the organic solvent used in the organic developing solution and then used. Typically, at least one solvent selected from a hydrocarbon-based solvent, a ketone-based solvent, an ester-based solvent, an alcohol-based solvent, an amide-based solvent, and an ether-based solvent is used.

These organic solvents may be used alone or a combination of two or more kinds thereof may be used. Further, an organic solvent other than the above-described solvents and water may be mixed and used.

The rinse treatment carried out using a rinse solution (washing treatment) can be performed according to a known rinse method. Examples of the method of performing the rinse treatment include a method of continuously ejecting a rinse solution onto a support rotating at a certain rate (rotary coating method), a method of immersing a support in a rinse solution for a certain time (dip method), and a method of spraying a rinse solution to the surface of a support (spray method).

According to the resist pattern formation method of the present embodiment described above, since the above-described resist composition is used, a resist pattern having excellent lithography characteristics such as LWR while maintaining satisfactory sensitivity can be formed.

It is preferable that various materials that are used in the resist composition according to the above-described embodiment and the pattern forming method according to the above-described embodiment (for example, a resist solvent, a developing solution, a rinse solution, a composition for forming an antireflection film, and a composition for forming a top coat) do not contain impurities such as a metal, a metal salt containing halogen, an acid, an alkali, and a component having a sulfur atom or phosphorus atom. Here, examples of the impurities containing metal atoms include Na, K, Ca, Fe, Cu, Mn, Mg, Al, Cr, Ni, Zn, Ag, Sn, Pb, Li, and salts thereof. The content of the impurities contained in these materials is preferably 200 ppb or less, more preferably 1 ppb or less, still more preferably 100 parts per trillion (ppt) or less, particularly preferably 10 ppt or less, and most preferably substantially zero (less than or equal to the detection limit of the measuring device).

### (Compound)

A compound according to a third aspect of the present invention is a compound represented by General Formula (b0) (compound (B0)).

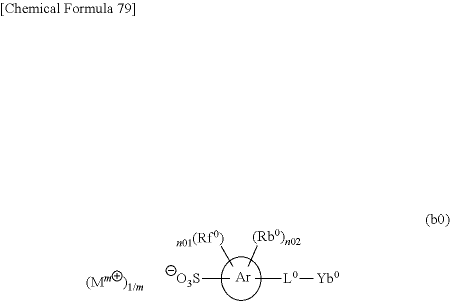

[In the formula, Ar represents an aromatic ring. Rf⁰ represents a fluorinated alkyl group having 1 to 5 carbon atoms or a fluorine atom. L⁰ represents a divalent linking group having —C(═O)—O—, —O—C(═O)—, or —O—S(═O)₂—. Yb⁰ represents a cyclic group. Rb⁰ represents an organic group. n01 represents an integer of 1 or greater as long as a valence is allowed. n02 represents an integer of 0 or greater as long as a valence is allowed. Here, in a case where L⁰-Yb⁰ represents —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of an aromatic ring is substituted with an alkyl group or an alkoxy group. In a case where n01 represents 2 or greater, a plurality of Rf⁰'s may be the same as or different from each other. In a case where n02 represents 2 or greater, a plurality of Rb⁰'s may be the same as or different from each other. m represents an integer of 1 or greater, and M^{m+} represents an m-valent cation.]

Ar, Rf⁰, L⁰, Rb⁰, n01, n02, and M^{m+} in Formula (b0) each have the same definition as described above. The compound of the present embodiment is preferably a compound represented by Formula (b0-1) and more preferably a compound represented by Formula (b0-1-1). Specific examples of the compound according to the present embodiment include a compound represented by any of Formulae (B0-1-an) to (B0-17-an). Specific examples of the compound according to the present embodiment further include a compound represented by any of Formulae (B0-1) to (B0-21).

### <Method for Producing Compound (B0)>

The compound (B0) according to the present embodiment can be produced by combining known methods.

The compound in which L⁰ in Formula (b0) represents a divalent linking group having —C(═O)—O— (hereinafter, also referred to as “compound (B0-a)”) can be obtained by the following reaction (Ia) and reaction (IIa).

### Reaction (Ia):

In the reaction (Ia), a compound represented by General Formula (Bpre0-a) is obtained by a reaction between a compound represented by General Formula (Xa) and a compound represented by General Formula (Ya).

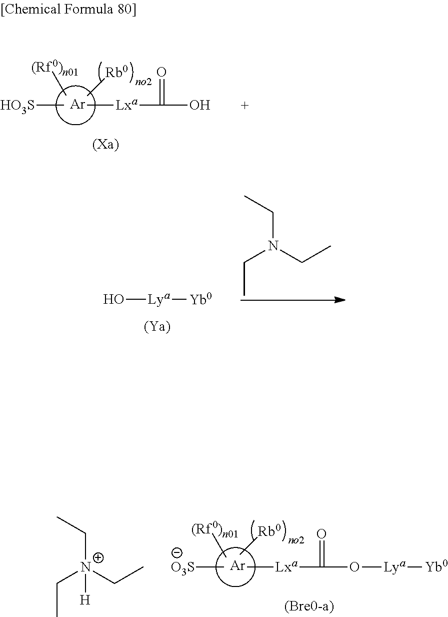

[In the reaction formula, Ar, Rf⁰, Rb⁰, Yb⁰, n01, and n02 each have the same definition as that for Ar, Rf⁰, Rb⁰, Yb⁰, n01, and n02 in Formula (b0). Lx^{a} and Ly^{a} each independently represent a single bond or a divalent linking group.]

As the divalent linking group represented by Lx^{a} and Ly^{a}, an alkylene group having 1 to 5 carbon atoms is preferable, and examples thereof include the same groups as those for Lb⁰¹ in Formula (L0-1).

### Reaction (IIa):

In the reaction (IIa), a salt exchange reaction between the compound represented by General Formula (Bre0-a) and the compound represented by General Formula (Z) is carried out to obtain a compound (B0-a).

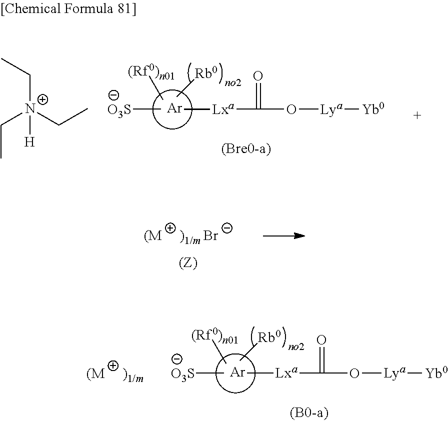

[In the reaction formula, Ar, Rf⁰, Rb⁰, Yb⁰, n01, and n02 each have the same definition as that for Ar, Rf⁰, Rb⁰, Yb⁰, n01, and n02 in Formula (b0). Lx^{a} and Ly^{a} each independently represent a single bond or a divalent linking group.]

The compound in which L⁰ in Formula (b0) represents a divalent linking group having —O—S(═O)— (hereinafter, also referred to as “compound (B0-b)”) can be obtained by the following reaction (Ib) and reaction (IIb).

### Reaction (Ib):

In the reaction (Ib), a compound represented by General Formula (Bpre0-b) is obtained by a reaction between a compound represented by General Formula (Xb) and a compound represented by General Formula (Yb).

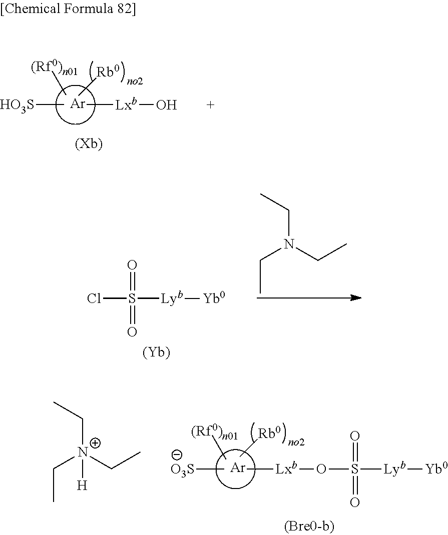

[In the reaction formula, Ar, Rf⁰, Rb⁰, Yb⁰, n01, and n02 each have the same definition as that for Ar, Rf⁰, Rb⁰, Yb⁰, n01, and n02 in Formula (b0). Lx^{b} and Ly^{b} each independently represent a single bond or a divalent linking group.]

As the divalent linking group represented by Lx^{b} and Ly^{b}, an alkylene group having 1 to 5 carbon atoms is preferable, and examples thereof include the same groups as those for Lb⁰¹ in Formula (L0-1).

### Reaction (IIb):

In the reaction (IIa), a compound (B0-b) is obtained by performing a salt exchange reaction between a compound represented by General Formula (Bre0-b) and a compound represented by General Formula (Z).

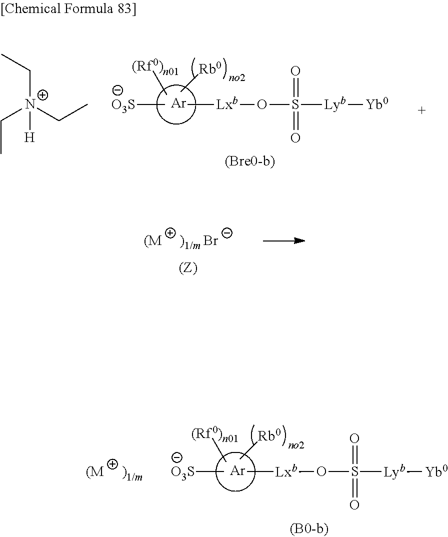

[In the reaction formula, Ar, Rf⁰, Rb⁰, Yb⁰, n01, and n02 each have the same definition as that for Ar, Rf⁰, Rb⁰, Yb⁰, n01, and n02 in Formula (b0). Lx^{a} and Ly^{a} each independently represent a single bond or a divalent linking group.]

The compound in which L⁰ in Formula (b0) represents a divalent linking group having —O—C(═O)— (hereinafter, also referred to as “compound (B0-c)”) can be obtained by the following reaction (Ic) and reaction (IIc).

### Reaction (Ic):

In the reaction (Ic), a compound represented by General Formula (Bpre0-c) is obtained by a reaction between a compound represented by General Formula (Xc) and a compound represented by General Formula (Yc).

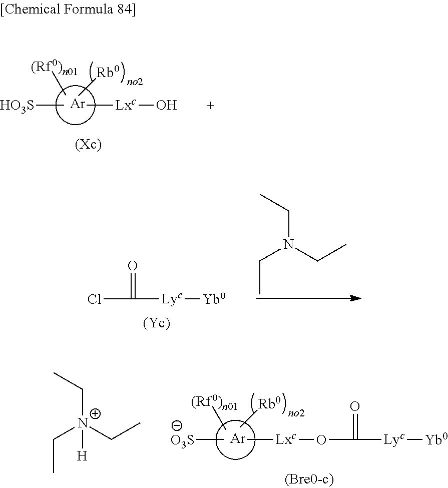

[In the reaction formula, Ar, Rf⁰, Rb⁰, Yb⁰, n01, and n02 each have the same definition as that for Ar, Rf⁰, Rb⁰, Yb⁰, n01, and n02 in Formula (b0). Lx^{c} and Ly^{c} each independently represent a single bond or a divalent linking group.]

As the divalent linking group represented by Lx^{c} and Ly^{c}, an alkylene group having 1 to 5 carbon atoms is preferable, and examples thereof include the same groups as those for Lb⁰¹ in Formula (L0-1).

### Reaction (IIc):

In the reaction (IIc), a compound (B0-c) is obtained by performing a salt exchange reaction between a compound represented by General Formula (Bre0-c) and a compound represented by General Formula (Z).

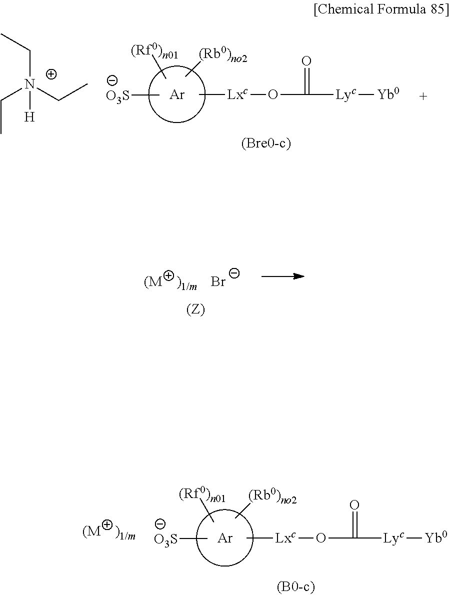

[In the reaction formula, Ar, Rf⁰, Rb⁰, Yb⁰, n01, and n02 each have the same definition as that for Ar, Rf⁰, Rb⁰, Yb⁰, n01, and n02 in Formula (b0). Lx^{c} and Ly^{c} each independently represent a single bond or a divalent linking group.]

The temperature conditions for the reaction (Ia) are not particularly limited, and are, for example, about −10° C. to 120° C., preferably in a range of 0° C. to 100° C., and more preferably in a range of 10° C. to 70° C.

The reaction time of the reaction (Ia) is not particularly limited, and is, for example, about 1 to 72 hours and preferably in a range of 1 to 24 hours.

Examples of the reaction solvent used in the reaction (Ia) include dichloromethane, dichloroethane, chloroform, tetrahydrofuran, N,N-dimethylformamide, acetonitrile, propionitrile, N,N′-dimethylacetamide, and dimethyl sulfoxide.

The condensation reaction in the reaction (la) may be carried out in the presence of a condensing agent.

Specific examples of the condensing agent include N,N′-dicyclohexylcarbodiimide, N,N′-diisopropylcarbodiimide (DIC), 1-ethyl-3-(3-dimethylaminopropyl) carbodiimide hydrochloride, and carbonyldiimidazole (CDI).

A basic catalyst may be used in the reaction (Ia).

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

The temperature conditions of the reactions (Ib) and (Ic) are not particularly limited, and are, for example, about −10° C. to 50° C., preferably in a range of 0° C. to 20° C., and more preferably in a range of 0° C. to 10° C.

The reaction time of the reactions (Ib) and (Ic) is not particularly limited, and is, for example, about 1 to 72 hours and preferably in a range of 1 to 24 hours.

Examples of the reaction solvent used in the reactions (Ib) and (Ic) described above include dichloromethane, dichloroethane, chloroform, tetrahydrofuran, N,N-dimethylformamide, acetonitrile, propionitrile, N,N′-dimethylacetamide, and dimethyl sulfoxide.

A basic catalyst may be used in the reactions (Ib) and (lc).

Specific examples of the basic catalyst include tertiary amines such as trimethylamine, triethylamine, and tributylamine, aromatic amines such as pyridine, dimethylaminopyridine (DMAP), and pyrrolidinopyridine, diazabicyclononene (DBN), and diazabicycloundecene (DBU).

The temperature conditions for the reactions (IIa) to (IIc) are not particularly limited, and are, for example, about 0° C. to 50° C.

The reaction time of the reactions (IIa) to (IIc) is not particularly limited, and is, for example, about 1 minute to 24 hours.

As the reaction solvent for the reactions (IIa) to (IIc), for example, a mixed solvent of an organic solvent and water is preferable. Examples of the organic solvent include a ketone-based solvent such as cyclohexanone, methyl ethyl ketone, or diethyl ketone, an ether-based solvent such as diethyl ether, t-butyl methyl ether, or diisopropyl ether, a halogen-based solvent such as tetrahydrofuran, 1,3-dioxolane, dichloromethane (methylene chloride), or 1,2-dichloroethane, an ester-based solvent such as ethyl acetate or propylene glycol monomethyl ether acetate, propionitrile, and a mixed solvent thereof.

After the completion of the reactions (IIa) to (IIc), the compound (B0) in the reaction solution may be isolated and purified.

Known methods of the related art can be used for the isolation and the purification, and for example, any one or a combination of two or more of concentrations, solvent extraction, distillation, crystallization, recrystallization, chromatography, and the like can be used.

The structure of the compound obtained as described above can be identified by typical organic analysis methods such as ¹H-nuclear magnetic resonance (NMR) spectroscopy, ¹³C-NMR spectroscopy, ¹⁹F-NMR spectroscopy, infrared (IR) absorption spectroscopy, mass spectrometry (MS), elemental analysis, and X-ray crystal diffraction.

The compound according to the present embodiment can be used as an acid generator according to a fourth aspect described below.

### (Acid Generator)

The acid generator according to the fourth aspect of the present invention contains the compound (compound (B0)) according to the third aspect.

The acid generator of the present embodiment can be used for the production of the resist composition according to the first aspect.

### EXAMPLES

Hereinafter, the present invention will be described in more detail based on examples, but the present invention is not limited to these examples.

### Synthesis Example of Compound

### Synthesis Example 1: Compound (B0-1)

20.0 g of a compound (X1), 13.3 g of a compound (Y1), 18.5 g of triethylamine, and 100 g of dichloromethane were added to a three-neck flask in a nitrogen atmosphere, and the mixture was stirred at 10° C. or lower. 12.0 g of diisopropylcarbodiimide was added thereto while the temperature was maintained, and the mixture was stirred at 10° C. or lower for 0.5 hours, heated to 25° C., and stirred for 12 hours. Thereafter, the reaction solution was filtered, and the filtrate was washed with 100 g of a 1% hydrochloric acid aqueous solution and washed with 100 g of water four times. After the filtrate was washed, the mixture was added dropwise to 500 g of hexane while being stirred, and the mixture was stirred for 30 minutes and filtered. The obtained powder was dried, thereby obtaining 20.4 g of a compound (Bpre-1).

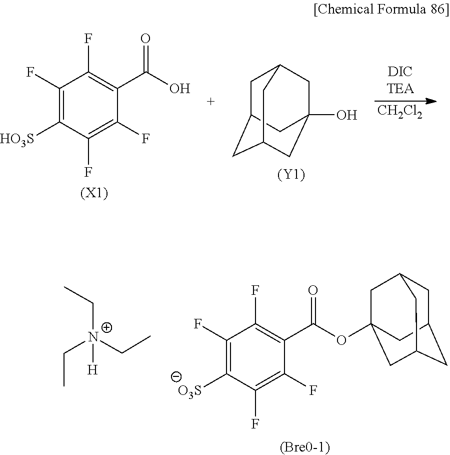

A precursor (Bpre0-1) (20 g, 58 mmol) and a compound Z (28 g, 55 mmol) were dissolved in dichloromethane (80 g), and ultrapure water (80 g) was added thereto to carry out a reaction at room temperature for 30 minutes. After completion of the reaction, the aqueous phase was removed, and the organic phase was washed with ultrapure water (80 g) four times. The organic phase was concentrated and dried using a rotary evaporator, thereby obtaining a compound (B0-1) (30 g, yield=82%).

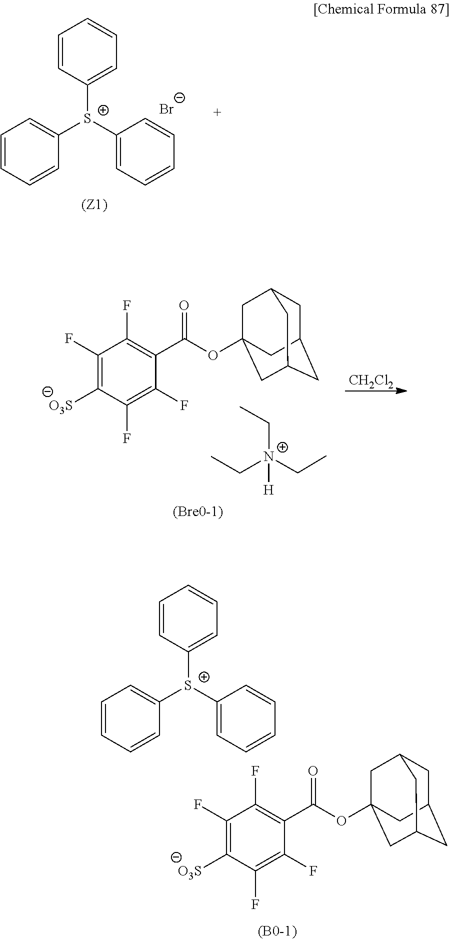

The obtained compound (B0-1) was subjected to NMR measurement, and the structure thereof was identified from the following analysis results.

¹H-NMR (DMSO, 400 MHz): δ (ppm)=1.76 (m, CH₂, 6H), 1.87 (m, CH, 3H), 2.00-2.11 (m, CH₂, 6H), 7.74-7.90 (m, CH, 15H)

### Synthesis Example 2: Compound (B0-2)

A compound (B0-2) was obtained by the same method as in Synthesis Example 1 except that the compound (Y1) was changed to a compound (Y2).

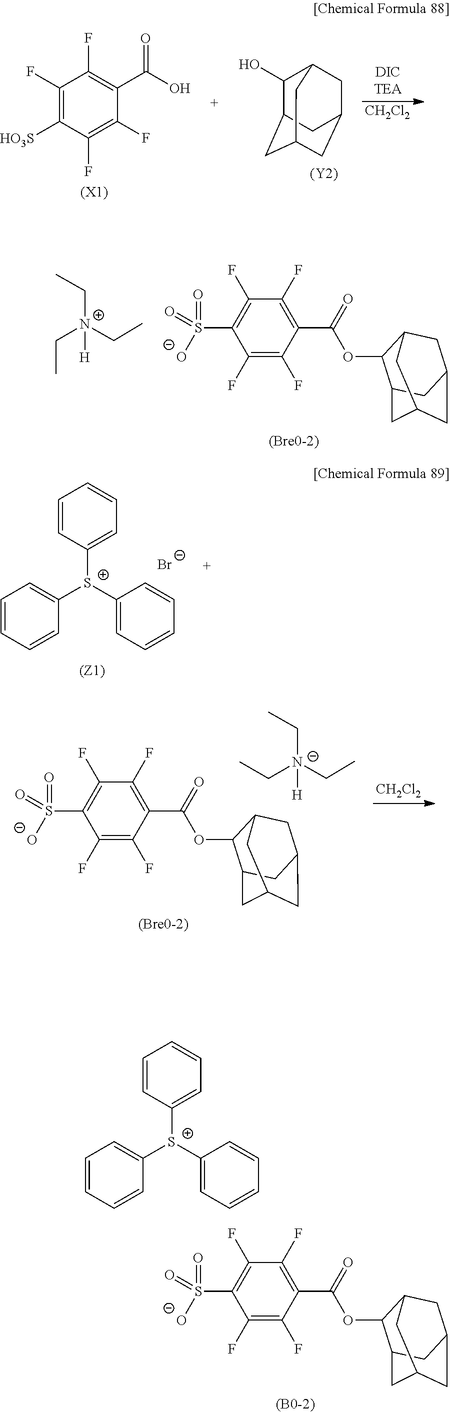

The obtained compound (B0-2) was subjected to NMR measurement, and the structure thereof was identified from the following analysis results.

¹H-NMR (DMSO, 400 MHz): δ (ppm)=1.76 (m, CH₂, 10H), 1.87 (m, CH, 3H), 2.50 (m, CH, 2H), 7.74-7.90 (m, CH, 15H)

### Synthesis Example 3: Compound (B0-3)

A compound (B0-3) was obtained by the same method as in Synthesis Example 1 except that the compound (Y1) was changed to a compound (Y3).

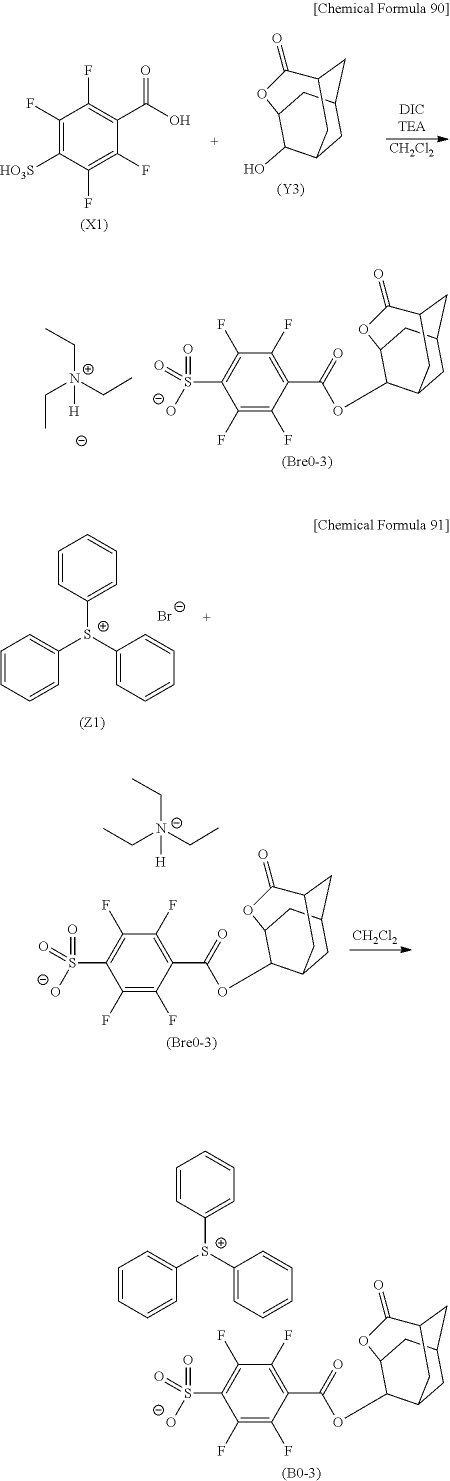

The obtained compound (B0-3) was subjected to NMR measurement, and the structure thereof was identified from the following analysis results.

¹H-NMR (DMSO, 400 MHz): δ (ppm)=1.10-1.32 (m, CH, 5H), 1.75 (m, CH₂, 4H), 2.00-2.35 (m, CH, 3H), 4.61 (m, CH, 1H), 7.74-7.90 (m, CH, 15H)

### Synthesis Example 4: Compound (B0-4)

A compound (B0-4) was obtained by the same method as in Synthesis Example 1 except that the compound (Y1) was changed to a compound (Y4).

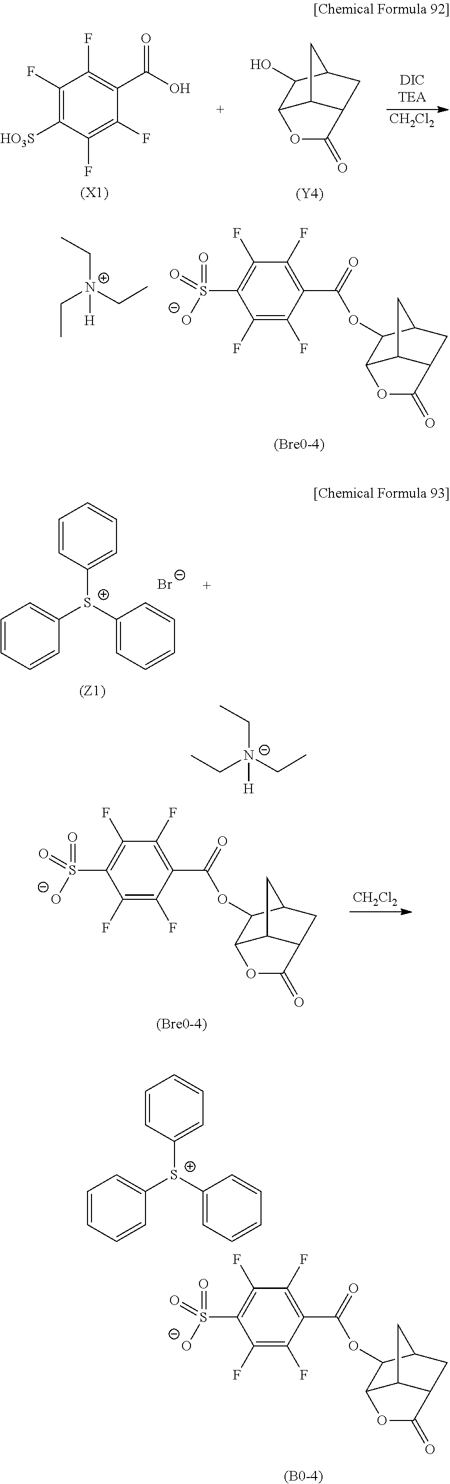

The obtained compound (B0-4) was subjected to NMR measurement, and the structure thereof was identified from the following analysis results.

¹H-NMR (DMSO, 400 MHz): δ (ppm)=1.60-1.86 (m, CH₂, 2H), 1.88-2.13 (m, CH₂+CH, 3H), 2.62-2.66 (m, CH, 2H), 5.10 (m, CH, 2H), 7.74-7.90 (m, CH, 15H)

### Synthesis Example 5: Compound (B0-5)

A compound (B0-5) was obtained by the same method as in Synthesis Example 1 except that the compound (Y1) was changed to a compound (Y5).

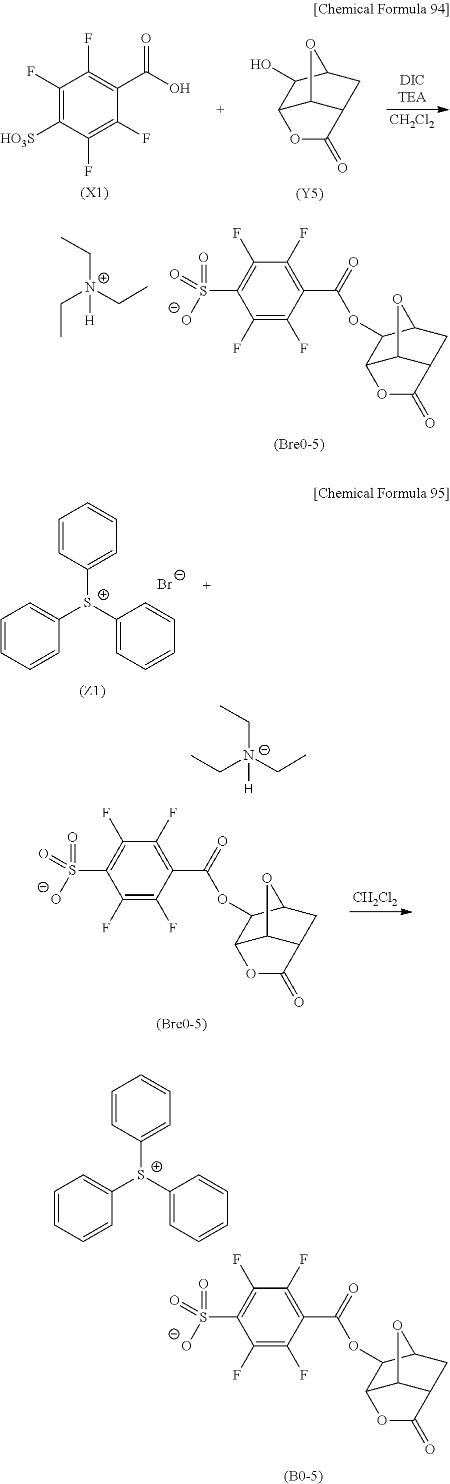

The obtained compound (B0-4) was subjected to NMR measurement, and the structure thereof was identified from the following analysis results.

¹H-NMR (DMSO, 400 MHz): δ (ppm)=1.79-2.04 (m, CH₂, 2H), 2.47 (m, CH, 1H), 3.83 (m, CH, 1H), 4.46 (m, CH, 1H), 5.40 (m, CH, 2H), 7.74-7.90 (m, CH, 15H)

### Synthesis Example 6: Compound (B0-6)

A compound (B0-6) was obtained by the same method as in Synthesis Example 1 except that the compound (Y1) was changed to a compound (Y6).

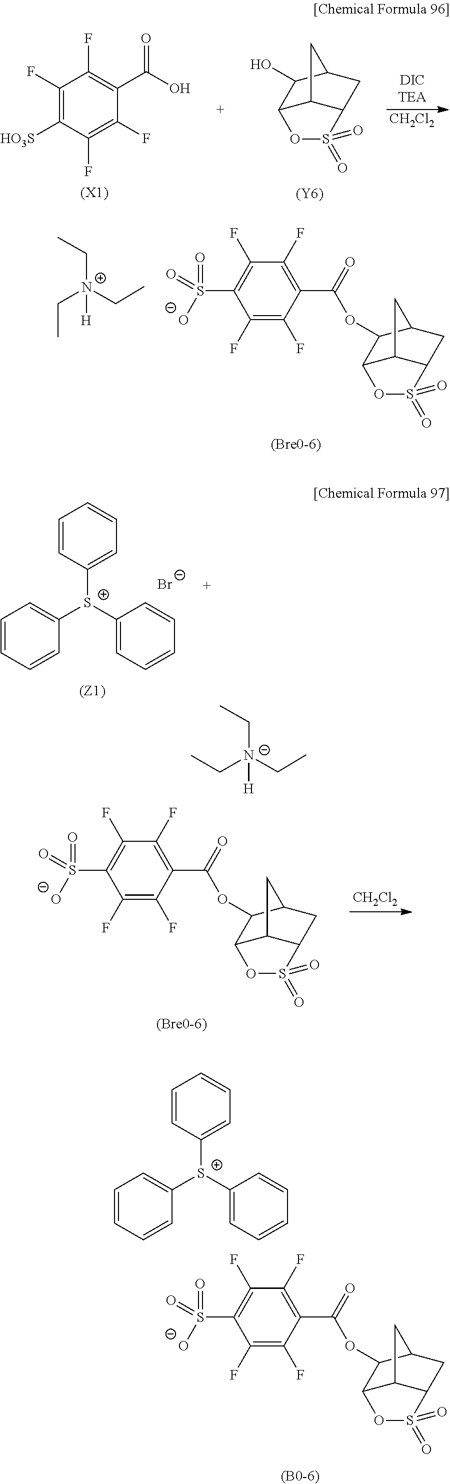

The obtained compound (B0-6) was subjected to NMR measurement, and the structure thereof was identified from the following analysis results.

¹H-NMR (DMSO, 400 MHz): δ (ppm)=1.60-1.91 (m, CH+CH₂, 3H), 2.13-2.25 (m, CH₂, 2H), 2.62-2.84 (m, CH, 2H), 4.65 (m, CH, 1H), 5.23 (m, CH, 1H), 7.74-7.90 (m, CH, 15H)

### Synthesis Example 7: Compound (B0-7)

A compound (B0-7) was obtained by the same method as in Synthesis Example 1 except that the compound (Y1) was changed to a compound (Y7).

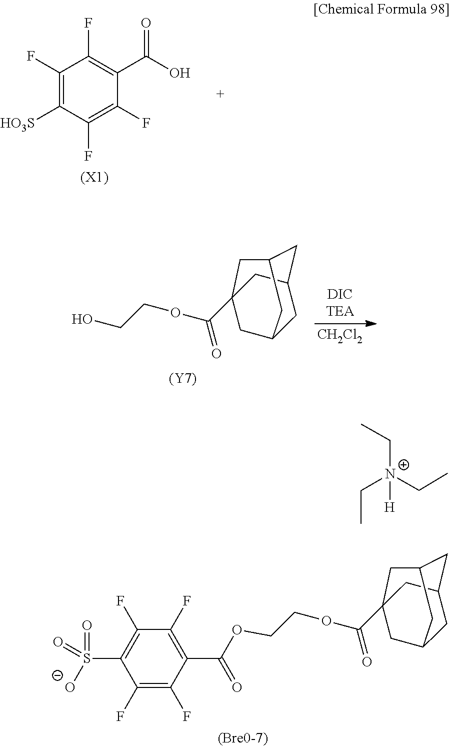

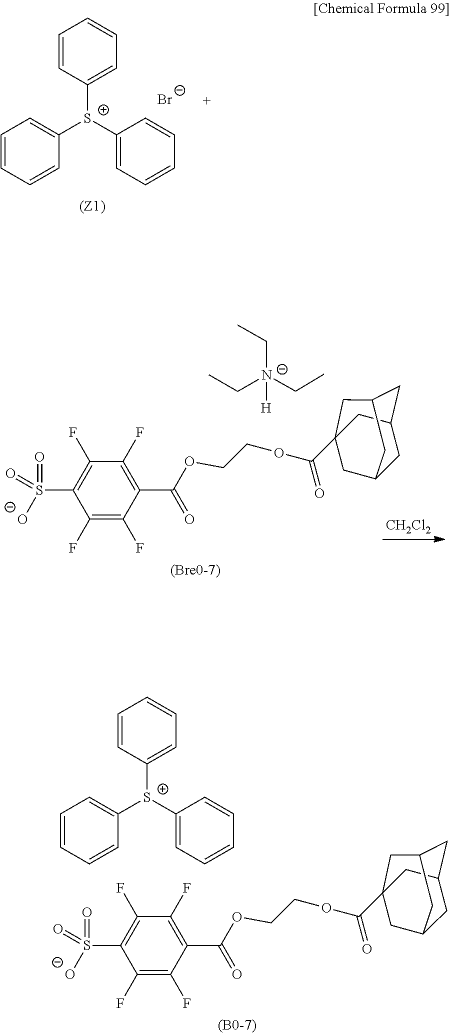

The obtained compound (B0-7) was subjected to NMR measurement, and the structure thereof was identified from the following analysis results.

¹H-NMR (DMSO, 400 MHz): δ (ppm)=1.87-2.11 (m, CH+CH₂, 15H), 4.21 (m, 2H, 2H), 4.46 (m, 2H, 2H), 7.74-7.90 (m, CH, 15H)

### Synthesis Example 8: Compound (B0-8)

15.2 g of a compound (X2), 14.4 g of triethylamine (TEA), and 60.8 g of dichloromethane (CH₂Cl₂) were added to a three-neck flask under a nitrogen atmosphere, and the mixture was stirred at 10° C. or lower. A solution of 14.4 g of a compound (Y8) dissolved in advance and 13.5 g of dichloromethane (CH₂Cl₂) was added thereto while the temperature of the mixture was maintained at 10° C. or lower, and the mixture was stirred at 10° C. or lower for 16 hours. Thereafter, the reaction solution was washed with 60.8 g of a 1% hydrochloric acid aqueous solution and washed with 60.8 g of water 4 times. After the reaction solution was washed, the solution was added dropwise to 304 g of hexane while being stirred, and the solution was stirred for 30 minutes and filtered. The obtained powder was dried, thereby obtaining 20.1 g of a compound (Bre0-8).

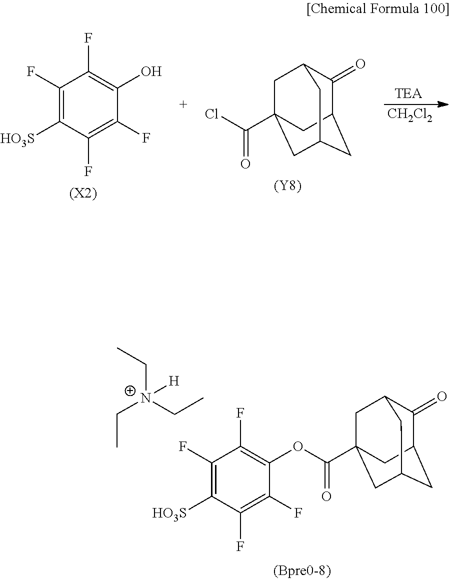

A precursor (Bpre0-8) (22 g, 42 mmol) and the compound Z (15 g, 44 mmol) were dissolved in dichloromethane (60 g), and ultrapure water (60 g) was added thereto to carry out a reaction at room temperature for 30 minutes. After completion of the reaction, the aqueous phase was removed, and the organic phase was washed with ultrapure water (60 g) four times. The organic phase was concentrated and dried using a rotary evaporator, thereby obtaining a compound (B0-8) (23 g, yield=79%).

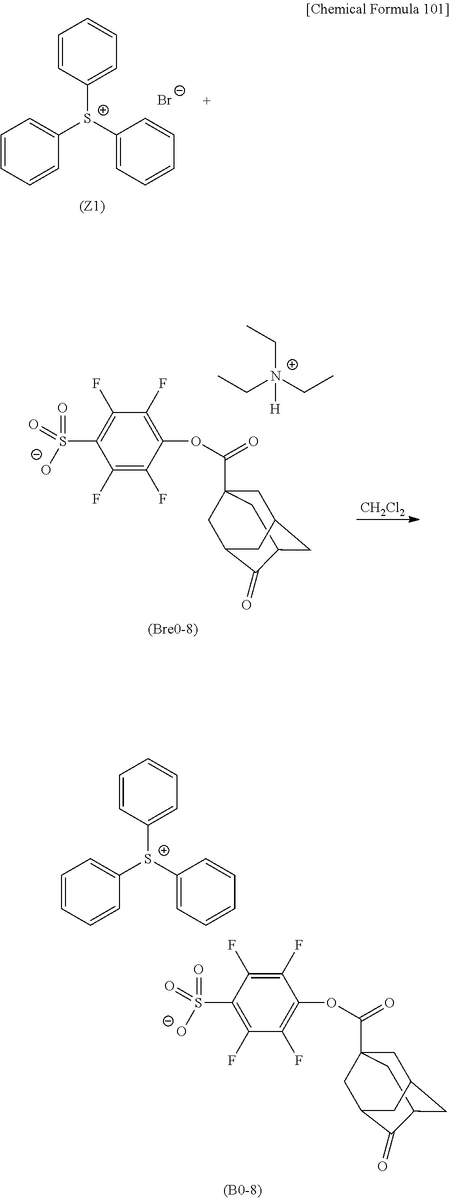

The obtained compound (B0-8) was subjected to NMR measurement, and the structure thereof was identified from the following analysis results.

¹H-NMR (DMSO, 400 MHz): δ (ppm)=1.10-2.30 (m, CH+CH₂, 13H), 7.74-7.90 (m, CH, 15H)

### Synthesis Example 9: Compound (B0-9)

A compound (B0-9) was obtained by the same method as in Synthesis Example 8 except that the compound (Y8) was changed to a compound (Y9).

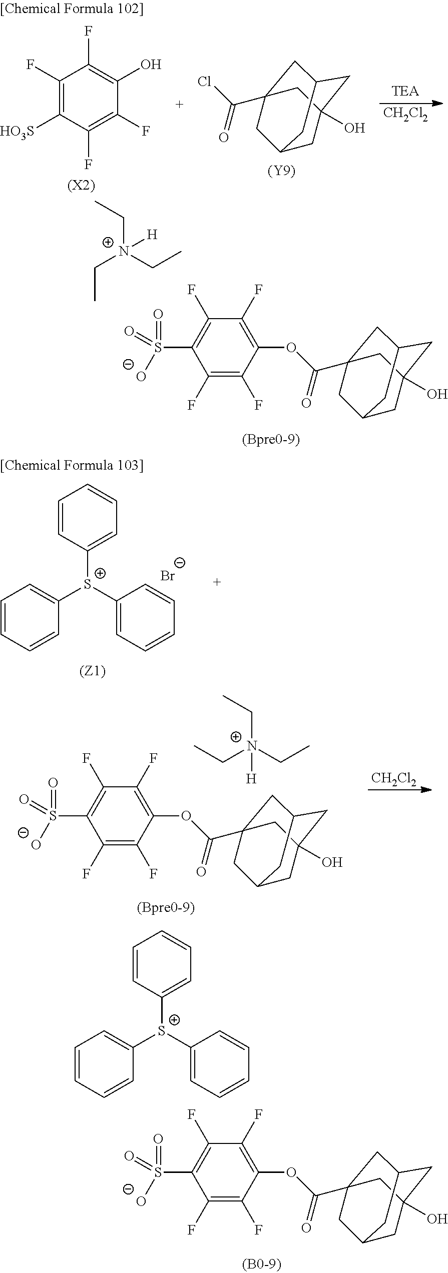

The obtained compound (B0-9) was subjected to NMR measurement, and the structure thereof was identified from the following analysis results.

¹H-NMR (DMSO, 400 MHz): δ (ppm)=0.87-1.79 (m, CH+CH₂, 14H), 1.97 (m, CH, 1H), 7.74-7.90 (m, CH, 15H)

### Synthesis Example 10: Compound (B0-10)

A compound (B0-10) was obtained by the same method as in Synthesis Example 8 except that the compound (Y8) was changed to a compound (Y10).

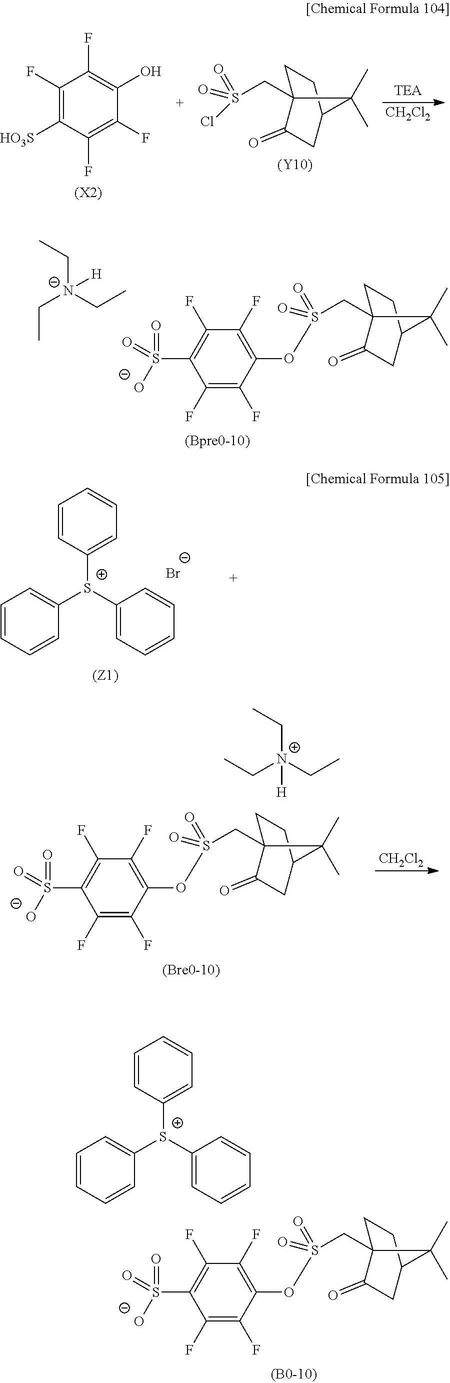

The obtained compound (B0-10) was subjected to NMR measurement, and the structure thereof was identified from the following analysis results.

¹H-NMR (DMSO, 400 MHz): δ (ppm)=0.96 (s, CH₃, 6H), 1.40-2.23 (m, CH+CH₂, 7H), 3.06-3.31 (m, CH₂, 2H), 7.74-7.90 (m, CH, 15H)

### Synthesis Example 11: Compound (B0-11)

A compound (B0-11) was obtained by the same method as in Synthesis Example 8 except that the compound (Y8) was changed to a compound (Y11).

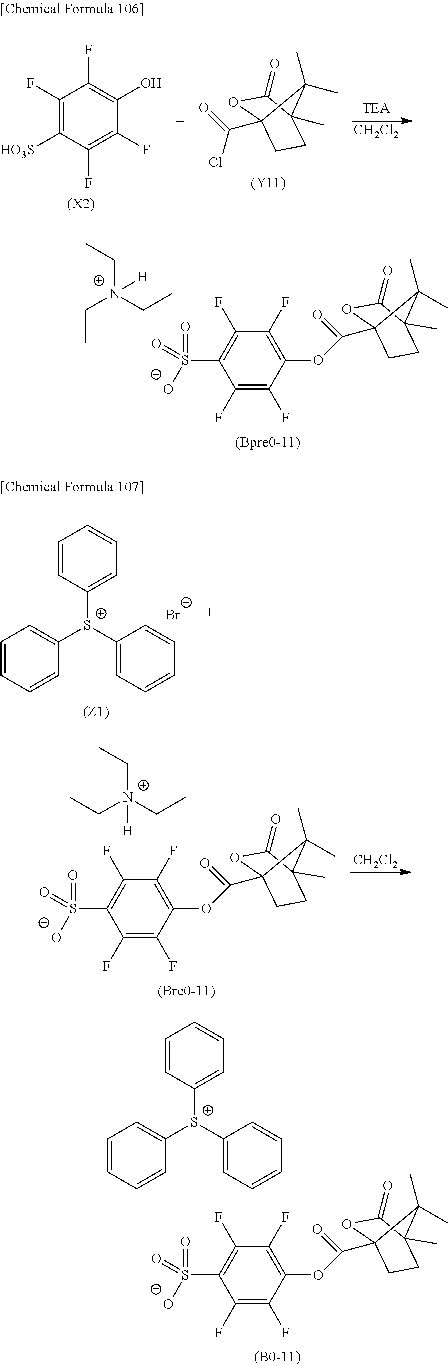

The obtained compound (B0-11) was subjected to NMR measurement, and the structure thereof was identified from the following analysis results.

¹H-NMR (DMSO, 400 MHz): δ (ppm)=0.89 (s, CH₃, 6H), 1.27 (s, CH₃, 3H), 1.76-2.23 (m, CH₂, 4H), 7.74-7.90 (m, CH, 15H)

### Synthesis Example 12: Compound (B0-12)

A compound (B0-11) was obtained by the same method as in Synthesis Example 8 except that the compound (Y8) was changed to a compound (Y12).

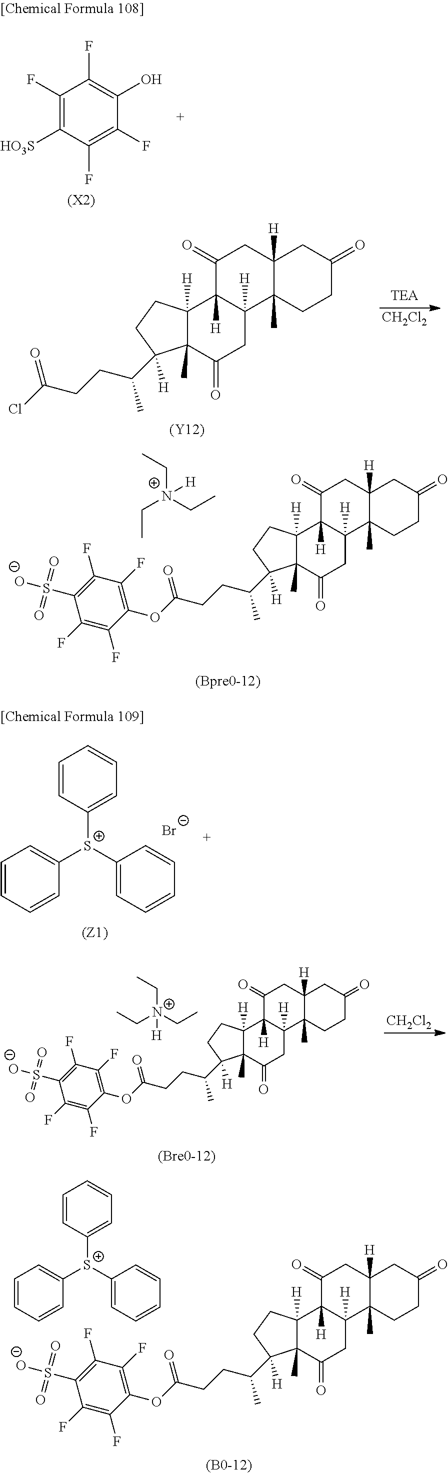

The obtained compound (B0-12) was subjected to NMR measurement, and the structure thereof was identified from the following analysis results.

¹H-NMR (DMSO, 400 MHz): δ (ppm)=0.84-0.88 (s, CH₃, 6H), 1.19 (s, CH₃, 3H), 1.19-2.55 (m, CH+CH₂, 24H), 7.74-7.90 (m, CH, 15H)

### Synthesis Example 13: Compound (B0-13)

A compound (B0-13) was obtained by the same method as in Synthesis Example 8 except that the compound (Y8) was changed to a compound (Y13).

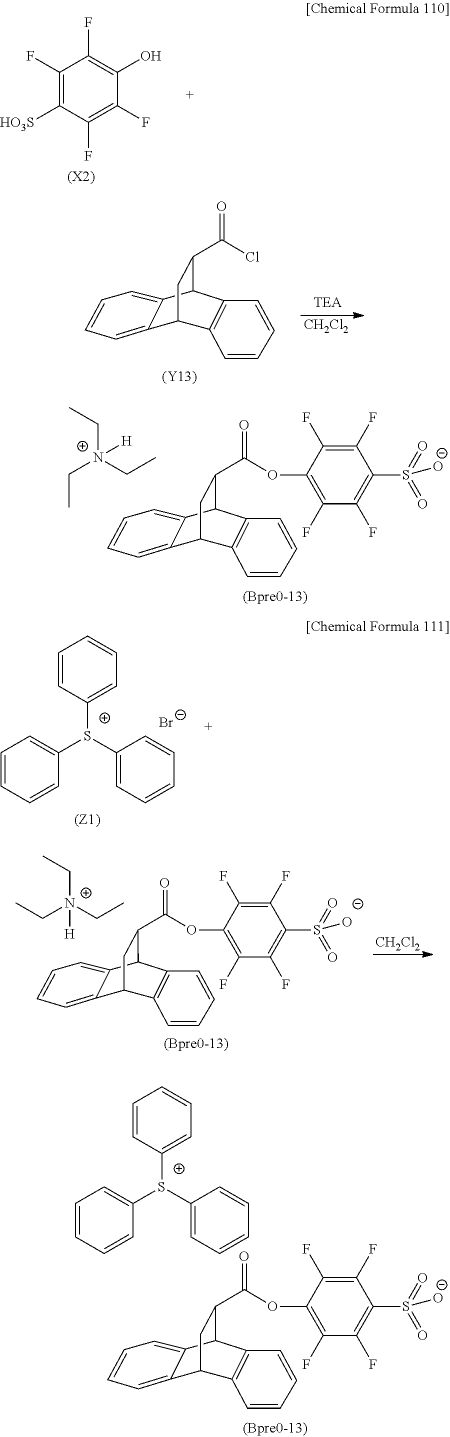

The obtained compound (B0-13) was subjected to NMR measurement, and the structure thereof was identified from the following analysis results.

¹H-NMR (DMSO, 400 MHz): δ (ppm)=2.11-2.37 (m, CH₂, 2H), 2.81 (m, CH, 1H), 3.81 (m, CH, 1H), 4.44 (m, CH, 1H), 1.19-7.31 (m, CH, 8H), 7.74-7.90 (m, CH, 15H)

### Synthesis Example 14: Compound (B0-14)

A compound (B0-14) was obtained by the same method as in Synthesis Example 8 except that the compound (Y8) was changed to a compound (Y14).

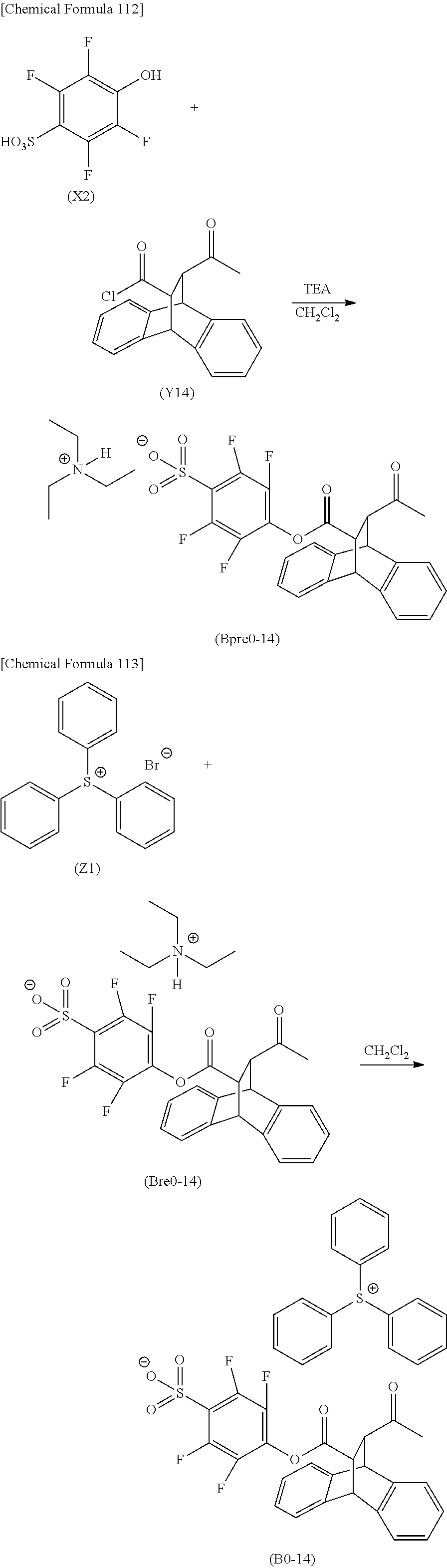

The obtained compound (B0-14) was subjected to NMR measurement, and the structure thereof was identified from the following analysis results.

¹H-NMR (DMSO, 400 MHz): δ (ppm)=(s, CH₃, 3H), 3.03 (m, CH, 1H), 3.47 (m, CH, 1H), 4.03 (m, CH, 1H), 4.44 (m, CH, 1H), 7.19-7.33 (m, CH, 8H), 7.74-7.90 (m, CH, 15H)

### Synthesis Example 15: Compound (B0-15)

A compound (B0-15) was obtained by the same method as in Synthesis Example 8 except that the compound (Y8) was changed to a compound (Y15).

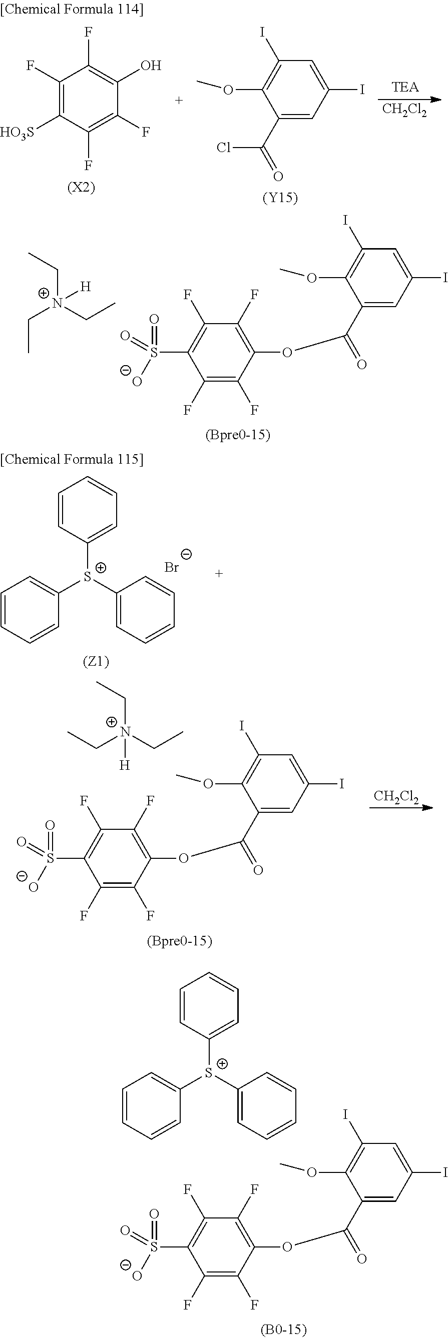

The obtained compound (B0-15) was subjected to NMR measurement, and the structure thereof was identified from the following analysis results.

¹H-NMR (DMSO, 400 MHz): δ (ppm)=3.83 (s, CH, 3H), 7.74-7.90 (m, CH, 15H), 8.27 (s, CH, 1H), 8.51 (s, CH, 1H)

### Synthesis Example 16: Compound (B0-16)

A compound (B0-16) was obtained by the same method as in Synthesis Example 6 except that the compound (Z1) was changed to (Z2).

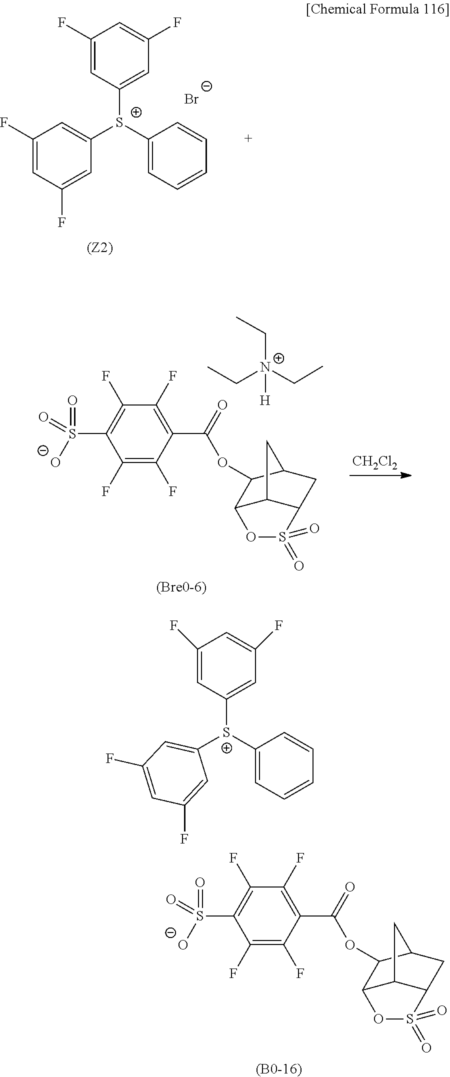

The obtained compound (B0-16) was subjected to NMR measurement, and the structure thereof was identified from the following analysis results.

¹H-NMR (DMSO, 400 MHz): δ (ppm)=1.60-1.91 (m, CH+CH₂, 3H), 2.13-2.25 (m, CH₂, 2H), 2.62-2.84 (m, CH, 2H), 4.65 (m, CH, 1H), 5.23 (m, CH, 1H), 7.77-7.98 (m, CH, 11H)

### Synthesis Example 17: Compound (B0-17)

A compound (B0-17) was obtained by the same method as in Synthesis Example 6 except that the compound (Z1) was changed to (Z3).

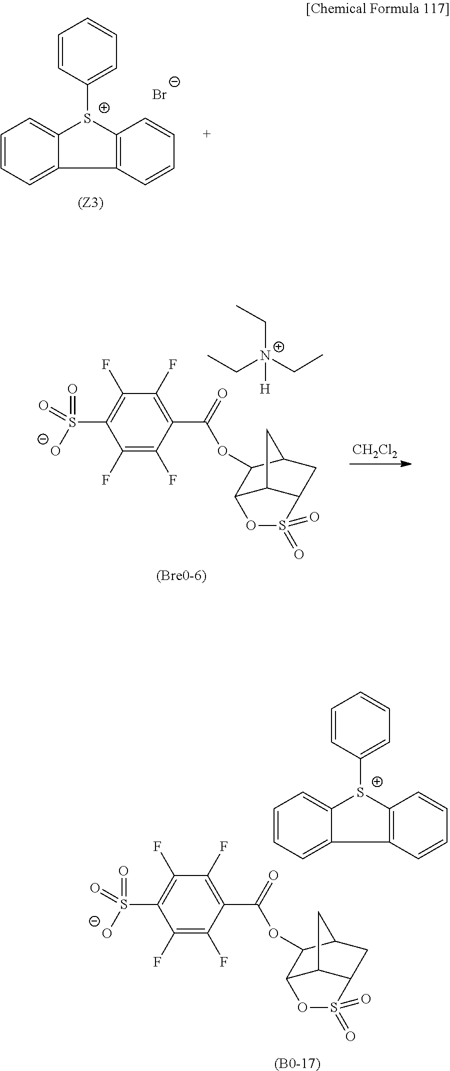

The obtained compound (B0-17) was subjected to NMR measurement, and the structure thereof was identified from the following analysis results.

¹H-NMR (DMSO, 400 MHz): δ (ppm)=1.60-1.91 (m, CH+CH₂, 3H), 2.13-2.25 (m, CH₂, 2H), 2.62-2.84 (m, CH, 2H), 4.65 (m, CH, 1H), 5.23 (m, CH, 1H), 7.55-7.75 (m, CH, 7H), 7.93 (t, CH, 2H), 8.37 (d, CH, 2H), 8.50 (d, CH, 2H)

### Synthesis Example 18: Compound (B0-18)

A compound (B0-18) was obtained by the same method as in Synthesis Example 13 except that the compound (Z1) was changed to the compound (Z2).

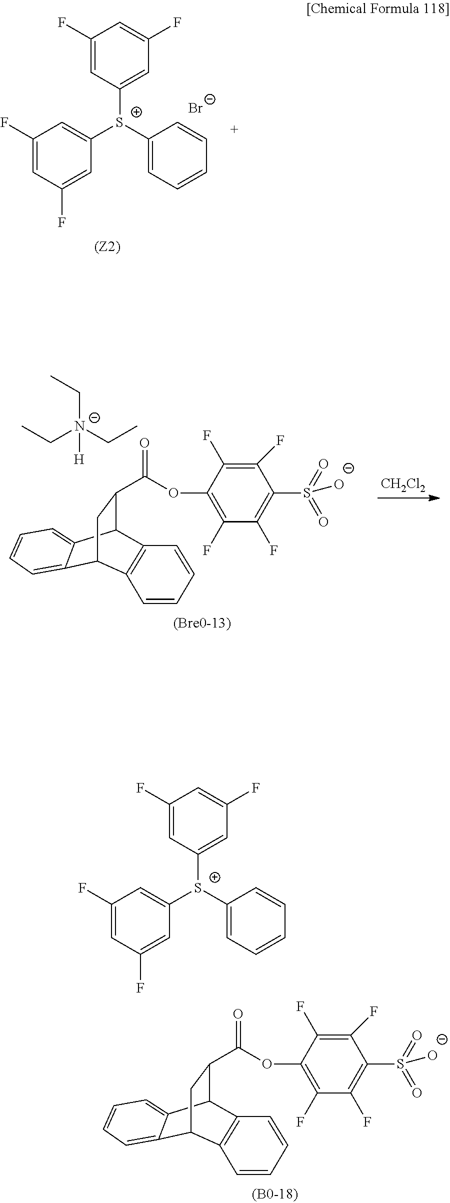

The obtained compound (B0-18) was subjected to NMR measurement, and the structure thereof was identified from the following analysis results.

¹H-NMR (DMSO, 400 MHz): δ (ppm)=2.11-2.37 (m, CH₂, 2H), 2.81 (m, CH, 1H), 3.81 (m, CH, 1H), 4.44 (m, CH, 1H), 1.19-7.31 (m, CH, 8H), 7.77-7.98 (m, Ph, 11H)

### Synthesis Example 19: Compound (B0-19)

A compound (B0-19) was obtained by the same method as in Synthesis Example 13 except that the compound (Z1) was changed to the compound (Z3).

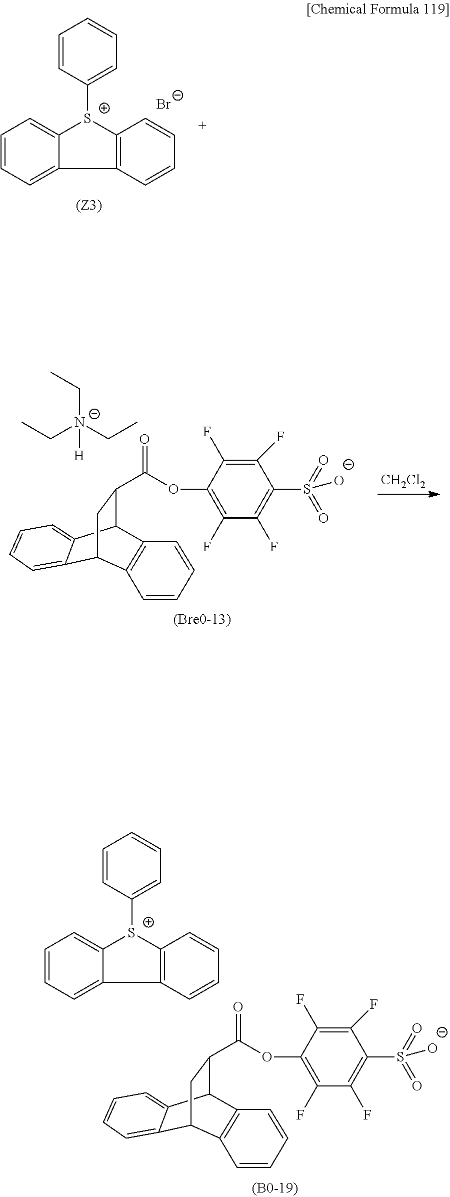

The obtained compound (B0-19) was subjected to NMR measurement, and the structure thereof was identified from the following analysis results.

¹H-NMR (DMSO, 400 MHz): δ (ppm)=2.11-2.37 (m, CH₂, 2H), 2.81 (m, CH, 1H), 3.81 (m, CH, 1H), 4.44 (m, CH, 1H), 1.19-7.31 (m, CH, 8H), 7.55-7.75 (m, CH, 7H), 7.93 (t, CH, 2H), 8.37 (d, CH, 2H), 8.50 (d, CH, 2H)

### Synthesis Example 20: Compound (B0-20)

A compound (B0-20) was obtained by the same method as in Synthesis Example 8 except that the compound (Y8) was changed to a compound (Y20).

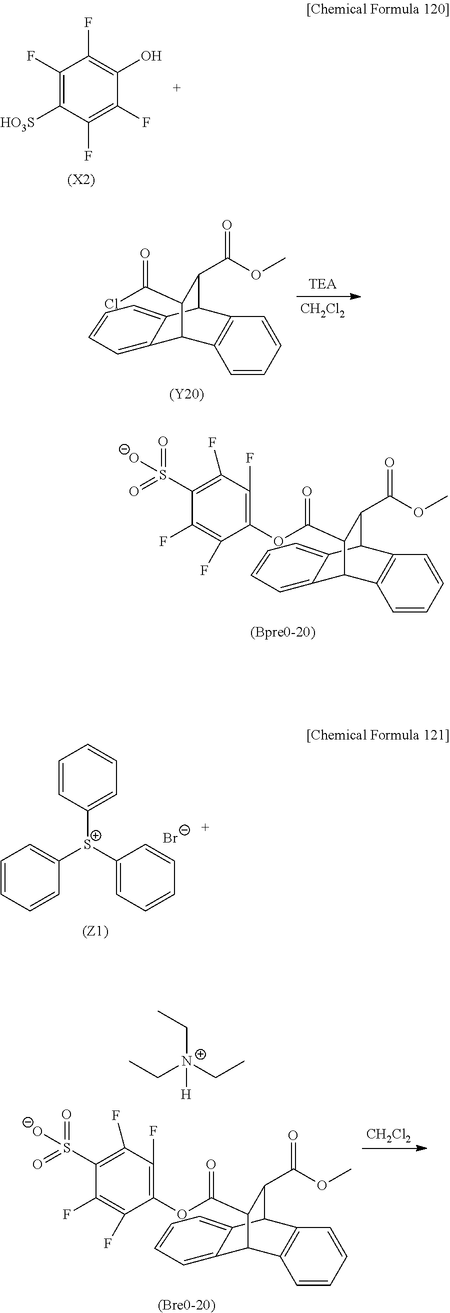

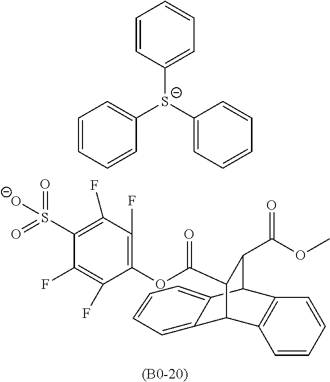

The obtained compound (B0-20) was subjected to NMR measurement, and the structure thereof was identified from the following analysis results.

¹H-NMR (DMSO, 400 MHz): δ (ppm)=3.44 (m, CH, 2H), 3.70 (s, CH₃, 3H), 4.44 (m, CH, 2H), 7.19-7.33 (m, CH, 8H), 7.74-7.90 (m, CH, 15H)

### Synthesis Example 21: Compound (B0-21)

A compound (B0-21) was obtained by the same method as in Synthesis Example 8 except that the compound (Y8) was changed to a compound (Y21).

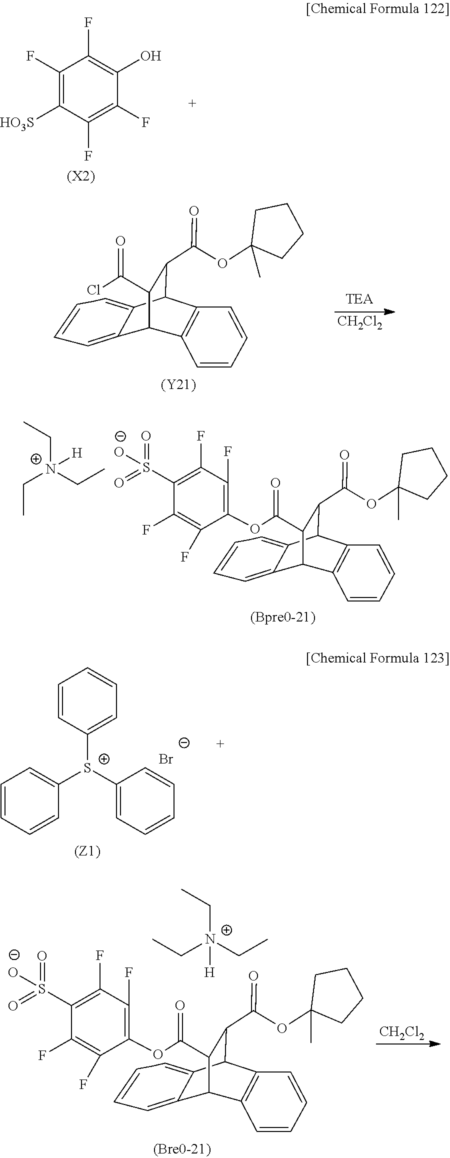

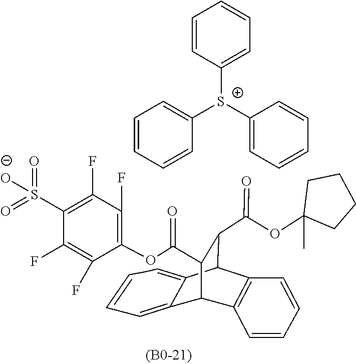 [SMILES: CC1(OC(=O)C2C3C4=CCC=CC=C4C(c4ccccc43)C2C(=O)Oc2c(F)c(F)c(S(=O)(=O)[O-])c(F)c2F)CCCC1 ; c1ccc([S+](c2ccccc2)c2ccccc2)cc1]

The obtained compound (B0-21) was subjected to NMR measurement, and the structure thereof was identified from the following analysis results.

¹H-NMR (DMSO, 400 MHz): δ (ppm)=1.39 (s, CH₃, 3H), 1.73-1.81 (m, CH₂, 8H), 3.44 (m, CH, 2H), 4.44 (m, CH, 2H), 7.19-7.33 (m, CH, 8H), 7.74-7.90 (m, CH, 15H)

### Preparation of Resist Composition

### Examples 1 to 25 and Comparative Examples 1 to 4

Each of the components listed in Table 1 to Table 3 was mixed and dissolved to prepare a resist composition of each example.

TABLE 1

| | Component | Component | Component | Component |
|---|---|---|---|---|
| | (A) | (B) | (D) | (S) |
| Example | (A1)-1 | (B0)-1 | (D1)-1 | (S)-1 |
| 1 | [100] | [24.0] | [9.5] | [8000] |
| Example | (A1)-1 | (B0)-2 | (D1)-1 | (S)-1 |
| 2 | [100] | [24.0] | [9.5] | [8000] |
| Example | (A1)-1 | (B0)-3 | (D1)-1 | (S)-1 |
| 3 | [100] | [25.1] | [9.5] | [8000] |
| Example | (A1)-1 | (B0)-4 | (D1)-1 | (S)-1 |
| 4 | [100] | [24.1] | [9.5] | [8000] |
| Example | (A1)-1 | (B0)-5 | (D1)-1 | (S)-1 |
| 5 | [100] | [24.1] | [9.5] | [8000] |
| Example | (A1)-1 | (B0)-6 | (D1)-1 | (S)-1 |
| 6 | [100] | [25.4] | [9.5] | [8000] |
| Example | (A1)-1 | (B0)-7 | (D1)-1 | (S)-1 |
| 7 | [100] | [26.6] | [9.5] | [8000] |
| Example | (A1)-1 | (B0)-8 | (D1)-1 | (S)-1 |
| 8 | [100] | [24.5] | [9.5] | [8000] |
| Example | (A1)-1 | (B0)-9 | (D1)-1 | (S)-1 |
| 9 | [100] | [24.6] | [9.5] | [8000] |
| Example | (A1)-1 | (B0)-10 | (D1)-1 | (S)-1 |
| 10 | [100] | [25.9] | [9.5] | [8000] |
| Example | (A1)-1 | (B0)-11 | (D1)-1 | (S)-1 |
| 11 | [100] | [24.6] | [9.5] | [8000] |
| Example | (A1)-1 | (B0)-12 | (D1)-1 | (S)-1 |
| 12 | [100] | [32.0] | [9.5] | [8000] |
| Example | (A1)-1 | (B0)-13 | (D1)-1 | (S)-1 |
| 13 | [100] | [27.0] | [9.5] | [8000] |

TABLE 2

| | Component | Component | Component | Component |
|---|---|---|---|---|
| | (A) | (B) | (D) | (S) |
| Example | (A1)-1 | (B0)-14 | (D1)-1 | (S)-1 |
| 14 | [100] | [28.0] | [9.5] | [8000] |
| Example | (A1)-1 | (B0)-15 | (D1)-1 | (S)-1 |
| 15 | [100] | [32.0] | [9.5] | [8000] |
| Example | (A1)-1 | (B0)-16 | (D1)-1 | (S)-1 |
| 16 | [100] | [27.9] | [9.5] | [8000] |
| Example | (A1)-1 | (B0)-17 | (D1)-1 | (S)-1 |
| 17 | [100] | [25.3] | [9.5] | [8000] |
| Example | (A1)-1 | (B0)-18 | (D1)-1 | (S)-1 |
| 18 | [100] | [29.6] | [9.5] | [8000] |
| Example | (A1)-1 | (B0)-19 | (D1)-1 | (S)-1 |
| 19 | [100] | [26.9] | [9.5] | [8000] |
| Example | (A1)-1 | (B0)-20 | (D1)-1 | (S)-1 |
| 20 | [100] | [22.5] | [9.5] | [8000] |
| Example | (A1)-1 | (B0)-21 | (D1)-1 | (S)-1 |
| 21 | [100] | [31.0] | [9.5] | [8000] |
| Example | (A1)-2 | (B0)-6 | (D1)-1 | (S)-1 |
| 22 | [100] | [25.4] | [9.5] | [8000] |
| Example | (A1)-3 | (B0)-6 | (D1)-1 | (S)-1 |
| 23 | [100] | [25.4] | [9.5] | [8000] |
| Example | (A1)-2 | (B0)-12 | (D1)-1 | (S)-1 |
| 24 | [100] | [32.0] | [9.5] | [8000] |
| Example | (A1)-3 | (B0)-12 | (D1)-1 | (S)-1 |
| 25 | [100] | [32.0] | [9.5] | [8000] |

TABLE 3

| | Component | Component | Component | Component |
|---|---|---|---|---|
| | (A) | (B) | (D) | (S) |
| Comparative | (A1)-1 | (B1)-1 | (D1)-1 | (S)-1 |
| Example | [100] | [23.0] | [9.5] | [8000] |
| 1 | | | | |
| Comparative | (A1)-1 | (B1)-2 | (D1)-1 | (S)-1 |
| Example | [100] | [21.9] | [9.5] | [8000] |
| 2 | | | | |
| Comparative | (A1)-1 | (B1)-3 | (D1)-1 | (S)-1 |
| Example | [100] | [35.4] | [9.5] | [8000] |
| 3 | | | | |
| Comparative | (A1)-1 | (B1)-4 | (D1)-1 | (S)-1 |
| Example | [100] | [22.2] | [9.5] | [8000] |
| 4 | | | | |

In Tables 1 to 3, each abbreviation has the following meaning. The numerical values in the brackets are blending amounts (parts by mass).
(A1)-1: polymer compound represented by Formula (A1-1). The weight-average molecular weight (Mw) determined by the GPC measurement in terms of standard polystyrene was 5800, and the polydispersity (Mw/Mn) thereof was 1.54. The copolymer compositional ratio (l/m) (the proportion (molar ratio) of each constitutional unit in the structural formula) determined by ¹³C-NMR was 50/50.
(A1)-2: polymer compound represented by Formula (A1-2). The weight-average molecular weight (Mw) determined by the GPC measurement in terms of standard polystyrene was 6000, and the polydispersity (Mw/Mn) thereof was 1.55. The copolymer compositional ratio (l/m) (the proportion (molar ratio) of each constitutional unit in the structural formula) determined by ¹³C-NMR was 50/50.
(A1)-3: polymer compound represented by Formula (A1-3). The weight-average molecular weight (Mw) determined by the GPC measurement in terms of standard polystyrene was 5900, and the polydispersity (Mw/Mn) thereof was 1.54. The copolymer compositional ratio (l/m) (the proportion (molar ratio) of each constitutional unit in the structural formula) determined by ¹³C-NMR was 50/50.

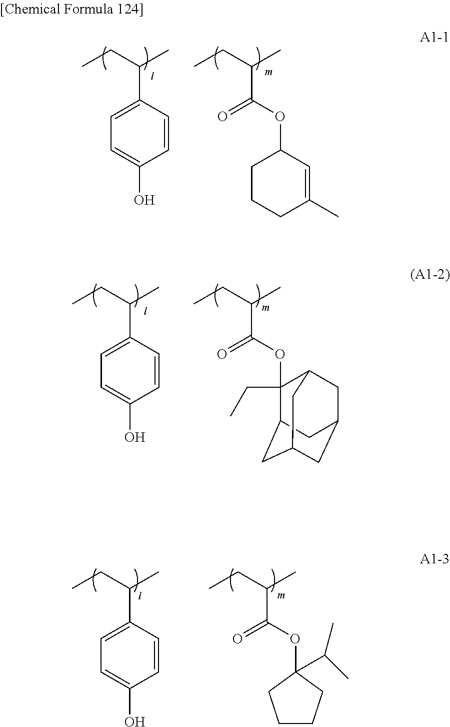

(B0)-1 to (B0)-21: acid generators formed of compounds (B0-1) to (B0-21) shown above.
(B1)-1 to (B1)-4: acid generators formed of compounds (B1-1) to (B1-4) shown above.

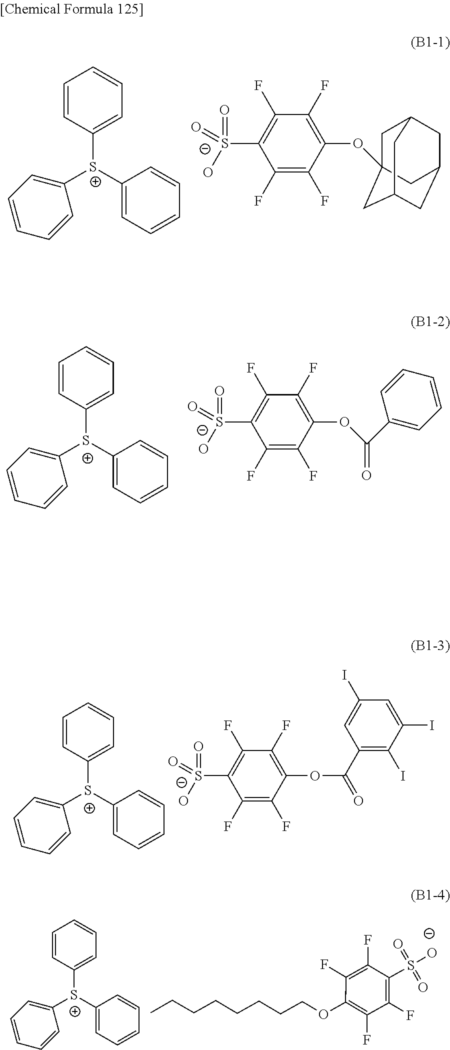

(D1)-1: acid diffusion control agent consisting of compound (D1-1) shown below.

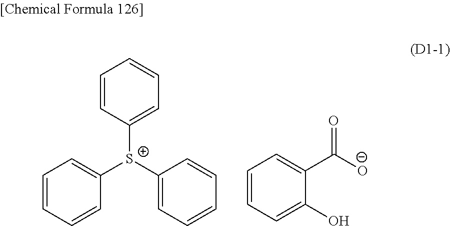 [SMILES: c1ccc([S+](c2ccccc2)c2ccccc2)cc1 ; O=C([O-])c1ccccc1O]

(S)-1: mixed solvent of propylene glycol monomethyl ether acetate/propylene glycol monomethyl ether=60/40 (mass ratio).

### Resist Pattern Formation

Step (i): An 8-inch silicon substrate which had been subjected to a hexamethyldisilazane (HMDS) treatment was coated with the resist composition of each example using a spinner, and subjected to a pre-bake (PAB) treatment on a hot plate at a temperature of 110° C. for 60 seconds so that the 8-inch silicon substrate was dried, thereby forming a resist film having a film thickness of 50 nm.
Step (ii): Next, drawing (light exposure) was performed on the resist film by using an electron beam lithography apparatus JEOL JBX-9300FS (manufactured by JEOL Ltd.) with the target size being set to a 1:1 line-and-space pattern (hereinafter, referred to as “LS pattern”) with a line width of 50 to 20 nm at an acceleration voltage of 100 kV. Thereafter, a post exposure bake (PEB) treatment was performed thereon at 100° C. for 60 seconds.
Step (iii): Next, alkali development was performed for 60 seconds using a 2.38 mass % tetramethylammonium hydroxide (TMAH) aqueous solution “NMD-3” (trade name, manufactured by Tokyo Ohka Kogyo Co., Ltd.) at 23° C. Thereafter, water rinsing was carried out with pure water for 15 seconds. As a result, a 1:1 LS pattern having a line width of 50 nm was formed.

### [Evaluation of Optimum Exposure Amount (Eop)]

According to <Resist pattern formation> described above, an optimum exposure amount Eop (μC/cm²) in which a pattern having the target size was formed was determined. The results thereof are listed in the columns of “EoP (μC/cm²)” in Tables 4 to 6.

### [Evaluation of Line Width Roughness (LWR)]

Using the LS pattern formed in the section of “formation of resist pattern”, the 3σ which is the scale that indicates the LWR was acquired. The results are listed in Tables 4 to 6 in the columns of “LWR (nm)”. Here, “3σ” denotes the triple value (3σ) (unit: nm) of the standard deviation (σ) determined from measurement results obtained by measuring 400 line positions in the longitudinal direction of the line with a scanning electron microscope (trade name: S-9380, manufactured by Hitachi High-Tech Corporation, acceleration voltage of 800 V). In a case where the value of the 3σ is small, this indicates that the roughness of a line side wall is small and an LS pattern with a uniform width is obtained.

TABLE 4

| | PAB | PEB | Eop | LWR |
|---|---|---|---|---|
| | (° C.) | (° C.) | (μC/cm²) | (nm) |
| Example 1 | 110 | 100 | 82 | 3.7 |
| Example 2 | 110 | 100 | 83 | 3.6 |
| Example 3 | 110 | 100 | 85 | 3.1 |
| Example 4 | 110 | 100 | 79 | 3.5 |
| Example 5 | 110 | 100 | 86 | 3.6 |
| Example 6 | 110 | 100 | 81 | 3.4 |
| Example 7 | 110 | 100 | 84 | 3.5 |
| Example 8 | 110 | 100 | 83 | 3.2 |
| Example 9 | 110 | 100 | 78 | 3.1 |
| Example 10 | 110 | 100 | 84 | 3.7 |
| Example 11 | 110 | 100 | 86 | 3.7 |
| Example 12 | 110 | 100 | 89 | 3.4 |
| Example 13 | 110 | 100 | 84 | 3.8 |

TABLE 5

| | PAB | PEB | Eop | LWR |
|---|---|---|---|---|
| | (° C.) | (° C.) | (μC/cm²) | (nm) |
| Example 14 | 110 | 100 | 82 | 3.2 |
| Example 15 | 110 | 100 | 87 | 3.3 |
| Example 16 | 110 | 100 | 83 | 3.2 |
| Example 17 | 110 | 100 | 85 | 3.5 |
| Example 18 | 110 | 100 | 84 | 3.6 |
| Example 19 | 110 | 100 | 87 | 3.2 |
| Example 20 | 110 | 100 | 82 | 3.2 |
| Example 21 | 110 | 100 | 88 | 3.3 |
| Example 22 | 110 | 100 | 95 | 3.8 |
| Example 23 | 110 | 100 | 91 | 3.2 |
| Example 24 | 110 | 100 | 99 | 3.5 |
| Example 25 | 110 | 100 | 93 | 3.2 |

TABLE 6

| | PAB | PEB | Eop | LWR |
|---|---|---|---|---|
| | (° C.) | (° C.) | (μC/cm²) | (nm) |
| Comparative | 110 | 100 | 89 | 4.5 |
| Example 1 | | | | |
| Comparative | 110 | 100 | 87 | 5.2 |
| Example 2 | | | | |
| Comparative | 110 | 100 | 88 | 4.9 |
| Example 3 | | | | |
| Comparative | 110 | 100 | 91 | 5.3 |
| Example 4 | | | | |

As listed in Tables 4 to 6, it was confirmed that the resist compositions of the examples can reduce LWR while maintaining satisfactory sensitivity as compared with the resist compositions of the comparative examples.

While preferred embodiments of the present invention have been described and illustrated above, it should be understood that these are exemplary examples of the present invention and are not to be considered as limiting. Additions, omissions, substitutions, and other modifications can be made without departing from the scope of the invention. Accordingly, the present invention is not limited by the description above but by the scope of the appended claims.

What is claimed is:

1. A resist composition which generates an acid upon light exposure and whose solubility in a developing solution is changed by an action of the acid, the resist composition comprising:
a resin component (A1) whose solubility in a developing solution is changed by the action of the acid; and
an acid generator component (B) which generates an acid upon light exposure,
wherein the acid generator component (B) contains a compound (B0) represented by General Formula (b0), 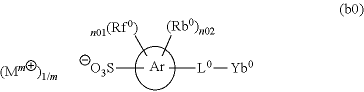
wherein Ar represents an aromatic ring, Rf⁰ represents a fluorinated alkyl group having 1 to 5 carbon atoms or a fluorine atom, L⁰ represents a divalent linking group having —C(═O)—O—, —O—C(═O)—, or —O—S(═O)₂—, Yb⁰ represents a cyclic group, Rb⁰ represents an organic group, n01 represents an integer of 1 or greater as long as a valence is allowed, n02 represents an integer of 0 or greater as long as a valence is allowed, wherein when L⁰-Yb⁰ represents —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, the aromatic hydrocarbon group is formed such that at least one hydrogen atom of an aromatic ring is substituted with an alkyl group or an alkoxy group, in a case where n01 represents 2 or greater, a plurality of Rf⁰'s may be the same as or different from each other, when n02 represents 2 or greater, a plurality of Rb⁰'s may be the same as or different from each other, m represents an integer of 1 or greater, and M^{m+} represents an m-valent cation.

2. The resist composition according to claim 1, wherein L⁰ in General Formula (b0) represents a divalent linking group having —C(═O)—O—, and Yb⁰ represents a cyclic group.

3. The resist composition according to claim 1, wherein L⁰-Yb⁰ in General Formula (b0) is —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, and the aromatic hydrocarbon group is formed such that at least one hydrogen atom of an aromatic ring is substituted with an alkyl group or an alkoxy group.

4. The resist composition according to claim 1, wherein L⁰ in General Formula (b0) represents a divalent linking group having —S(═O)₂—O—, and Yb⁰ represents a cyclic group.

5. The resist composition according to claim 1, wherein the compound (B0) is a compound represented by General Formula (b0-1), 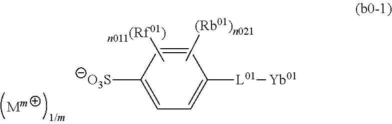
wherein Rf⁰¹ represents a fluorinated alkyl group having 1 to 5 carbon atoms or a fluorine atom, L⁰¹ represents a divalent linking group having —C(═O)—O—, —O—C(═O)—, or —O—S(═O)₂—, Yb⁰¹ represents a cyclic group, Rb⁰¹ represents an organic group, n011 represents an integer of 1 or greater, n021 represents an integer of 0 or greater, n011+n021≤4 is satisfied, wherein when L⁰¹-Yb⁰¹ represents —O—C(═O)—Yb⁰¹, Yb⁰¹ represents an alicyclic group which may have a substituent, a condensed cyclic group of an aliphatic ring and an aromatic ring, which may have a substituent, or an aromatic hydrocarbon group which has a substituent, the aromatic hydrocarbon group is formed such that at least one hydrogen atom of an aromatic ring is substituted with an alkyl group or an alkoxy group, in a case where n011 represents 2 or greater, a plurality of Rf⁰¹'s may be the same as or different from each other, when n021 represents 2 or greater, a plurality of Rb⁰¹'s may be the same as or different from each other, m represents an integer of 1 or greater, and M^{m+} represents an m-valent cation.

6. A resist pattern formation method comprising:
forming a resist film on a support using the resist composition according to claim 1;
exposing the resist film to light; and
developing the resist film exposed to light to form a resist pattern.

7. A compound which is represented by General Formula (b0), 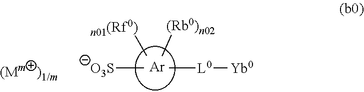
wherein Ar represents an aromatic ring, Rf⁰ represents a fluorinated alkyl group having 1 to 5 carbon atoms or a fluorine atom, L⁰ represents a divalent linking group having —C(═O)—O—, —O—C(═O)—, or —O—S(═O)₂—, Yb⁰ represents a cyclic group, Rb⁰ represents an organic group, n01 represents an integer of 1 or greater as long as a valence is allowed, n02 represents an integer of 0 or greater as long as a valence is allowed, wherein when L⁰-Yb⁰ represents —O—C(═O)—Yb⁰, Yb⁰ represents an alicyclic group which may have a substituent, a condensed cyclic group of an aliphatic ring and an aromatic ring, which may have a substituent, or an aromatic hydrocarbon group which has a substituent, the aromatic hydrocarbon group is formed such that at least one hydrogen atom of an aromatic ring is substituted with an alkyl group or an alkoxy group, in a case where n01 represents 2 or greater, a plurality of Rf⁰'s may be the same as or different from each other, when n02 represents 2 or greater, a plurality of Rb⁰'s may be the same as or different from each other, m represents an integer of 1 or greater, and M^{m+} represents an m-valent cation.

8. The compound according to claim 7, wherein the compound is represented by General Formula (b0-1), 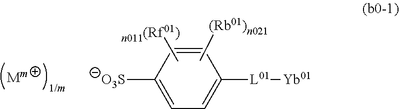
wherein Rf⁰¹ represents a fluorinated alkyl group having 1 to 5 carbon atoms or a fluorine atom, L⁰¹ represents a divalent linking group having —C(═O)—O—, —O—C(═O)—, or —O—S(═O)₂—, Yb⁰¹ represents a cyclic group, Rb⁰¹ represents an organic group, n011 represents an integer of 1 or greater, n021 represents an integer of 0 or greater, n011+n021≤4 is satisfied, wherein when L⁰¹-Yb⁰¹ represents —O—C(═O)—Yb⁰¹, Yb⁰¹ represents an alicyclic group which may have a substituent, a condensed cyclic group of an aliphatic ring and an aromatic ring, which may have a substituent, or an aromatic hydrocarbon group which has a substituent, the aromatic hydrocarbon group is formed such that at least one hydrogen atom of an aromatic ring is substituted with an alkyl group or an alkoxy group, in a case where n011 represents 2 or greater, a plurality of Rf⁰¹'s may be the same as or different from each other, when n021 represents 2 or greater, a plurality of Rb⁰¹'s may be the same as or different from each other, m represents an integer of 1 or greater, and M^{m+} represents an m-valent cation.

9. An acid generator comprising the compound according to claim 7.
